(19) United States
(12) Patent Application Publication
(10) Pub. No.: US 2022/0268780 A1
(43) Pub. Date: Aug. 25, 2022

(54) METHODS OF PREDICTING ENDOMETRIAL RECEPTIVITY

(71) Applicants: HUDSON INSTITUTE OF MEDICAL RESEARCH, Clayton (AU); MONASH IVF PTY LTD, Richmond (AU)

(72) Inventors: Guiying Nie, Glen Waverley (AU); Sophea Heng, Springvale (AU); Luk Johan Frans Rombauts, Glen Iris (AU)

(73) Assignees: HUDSON INSTITUTE OF MEDICAL RESEARCH, Clayton (AU); MONASH IVF PTY LTD, Richmond (AU)

(21) Appl. No.: 17/622,668

(22) PCT Filed: Jun. 25, 2020
(86) PCT No.: PCT/AU2020/050645

§ 371 Date: Dec. 23, 2021

(30) Foreign Application Priority Data
Jun. 25, 2019 (AU) 2019902204

(51) Int. Cl. G01N 33/68; C12Q 1/6883

(52) U.S. Cl. CPC G01N 33/689; C12Q 1/6883; G01N 2800/367; C12Q 2600/178; G01N 2333/70596; C12Q 2600/158

(57) ABSTRACT

The present invention relates to methods of predicting endometrial receptivity for embryo implantation in a subject, the method comprising determining a level of podocalyxin in endometrial epithelial cells in the subject. The present invention also relates to methods of monitoring epithelial receptivity and improving epithelial receptivity.

### RELATED APPLICATION DATA

The present application claims priority from Australian Patent Application No. 2019902204 entitled “Methods of predicting endometrial receptivity” filed on 25 Jun. 2019, the entire contents of which is hereby incorporated by reference.

### SEQUENCE LISTING

The present application is filed with a Sequence Listing in electronic form. The entire contents of the Sequence Listing are hereby incorporated by reference.

### FIELD OF THE INVENTION

The present disclosure relates to methods of predicting endometrial receptivity for embryo implantation in a subject, the method comprising determining a level of podocalyxin in endometrial epithelial cells in the subject. The present disclosure also provides methods of monitoring epithelial receptivity and improving epithelial receptivity.

### BACKGROUND OF THE INVENTION

Embryo implantation is a key step in establishing pregnancy, and implantation failure can cause infertility. Assisted reproductive technology (ART) is a major intervention to overcome infertility, however, low implantation rates (˜30% per average ART cycle) significantly limit ART success.

Implantation involves highly coordinated interactions between an embryo and the uterus. For implantation to succeed, the embryo has to be well-developed and capable of implantation, and the uterus has to be in a receptive state.

Innovations in embryo culture and selection have significantly improved ART in recent years. However, even with the latest embryo technologies, including preimplantation genetic screening, implantation failure still remains a limiting obstacle, highlighting the importance of the endometrium in determining implantation outcomes.

The inner lining of the uterus, the endometrium, participates in implantation, and the process of implantation differs greatly among species. Human implantation requires the embryo to attach to the endometrial luminal epithelium, traverse the epithelial layer, penetrate the underneath basement membrane, and eventually move to the stromal compartment. The luminal epithelium then reseals over the implantation site, completely encapsulating the embryo within the tissue. This human implantation cascade is unique and no animal model recapitulates all aspects of the human implantation process.

In every menstrual cycle, the human endometrium remodels substantially under the influence of ovarian hormones estrogen and progesterone, becoming receptive only in the mid-secretory phase (days 20-24 of a 28 day cycle) when progesterone is dominant. This synchronizes endometrial receptivity with embryo development for implantation.

However, the detailed molecular and cellular mechanisms that control endometrial receptivity remain to be fully elucidated. In particular, it is unknown how the luminal epithelium remodels for embryo attachment and invasion. Transcriptomic analyses of endometrial tissues have revealed a large number of genes up- or down-regulated at receptivity, though data sets vary greatly between studies. A microarray-based mRNA signature technology termed ERA (endometrial receptivity array) has been developed to identify the receptive window, although the utility of ERA is still being proven. In addition, ERA uses whole tissue biopsy and thus cannot pinpoint the specific involvement of a particular cell type or a specific molecule.

Accordingly, it will be clear to the skilled person that there is an on-going need in the art for the development of methods of predicting the optimal period for embryo implantation and reducing implantation failure.

### SUMMARY OF THE INVENTION

In producing the present disclosure, the inventors identified podocalyxin as a key negative regulator of human endometrial epithelial receptivity. The inventors studied the role of this regulator in human tissue samples and its association with implantation failure in IVF patients. Methods of modulating and regulating the expression of podocalyxin were also assessed. Surprisingly, the present inventors have found that down regulation of podocalyxin in the luminal but not glandular epithelial cells signifies epithelial receptivity.

The findings by the inventors provide the basis for methods of identifying or predicting endometrial receptivity for embryo implantation in a subject. For example, the present disclosure provides a method of predicting endometrial receptivity for embryo implantation in a subject, the method comprising determining a level of podocalyxin in endometrial epithelial cells in the subject.

In one example, the present disclosure provides a method of predicting endometrial epithelial receptivity for embryo implantation in a subject, the method comprising determining a level of podocalyxin in endometrial epithelial cells in the subject.

In one example, determining the level of podocalyxin comprises determining the amount and/or distribution pattern of podocalyxin protein, and/or determining the amount of nucleic acid molecules encoding podocalyxin, in the endometrial epithelial cells.

In one example, determining the level of podocalyxin comprises determining the amount and/or distribution pattern of podocalyxin protein in the endometrial epithelial cells. For example, determining the level of podocalyxin comprises determining the amount of podocalyxin protein in the endometrial epithelial cells. In another example, determining the level of podocalyxin comprises determining the distribution pattern of podocalyxin protein in the endometrial epithelial cells.

In one example, determining the level of podocalyxin comprises determining the amount of nucleic acid molecules encoding podocalyxin, in the endometrial epithelial cells.

In one example, the nucleic acid molecules are mRNA. Methods of measuring the amount of nucleic acid molecules in the endometrial epithelial cells are known in the art and/or are described herein. For example, the nucleic acid molecules are detected using real-time reverse transcription polymerase chain reaction (RT-PCR).

In one example, the method further comprises comparing the level of podocalyxin in the subject to a level of podocalyxin in endometrial epithelial cells in at least one reference. Methods of determining a reference will be apparent to the skilled person and/or are described herein.

In one example, the method comprises determining (a) if the level of the podocalyxin in the subject is higher than the level of the podocalyxin in the reference, or (b) if the level of the podocalyxin in the subject is lower than the level of podocalyxin in the reference.

In one example, the endometrial epithelial cells are luminal epithelial cells and/or glandular epithelial cells. For example, the endometrial epithelial cells are luminal epithelial cells. In another example, the endometrial epithelial cells are glandular epithelial cells.

In one example, the method of the disclosure provides:

(i) a lower level of podocalyxin in luminal epithelial cells and a higher level of podocalyxin in glandular epithelial cells of the subject is indicative of endometrial epithelial receptivity; or

(ii) a higher level of podocalyxin in luminal epithelial cells and a higher level of podocalyxin in glandular epithelial cells of the subject is indicative of pre-endometrial epithelial receptivity; or

(iii) a lower level of podocalyxin in luminal epithelial cells and a lower level of podocalyxin in glandular epithelial cells of the subject is indicative of post-endometrial epithelial receptivity.

In one example, a lower level of podocalyxin in luminal epithelial cells and a higher level of podocalyxin in glandular epithelial cells of the subject is indicative of endometrial epithelial receptivity.

In one example, a higher level of podocalyxin in luminal epithelial cells and a higher level of podocalyxin in glandular epithelial cells of the subject is indicative of pre-endometrial epithelial receptivity.

In one example, a lower level of podocalyxin in luminal epithelial cells and a lower level of podocalyxin in glandular epithelial cells of the subject is indicative of post-endometrial epithelial receptivity.

In one example, the method comprises using an antibody or aptamer that specifically binds podocalyxin to determine the level of podocalyxin. For example, the method comprises using an antibody that specifically binds podocalyxin to determine the level of podocalyxin. Antibodies suitable for use in the present disclosure will be apparent to the skilled person and/or are described herein. In another example, the method comprises using an aptamer that specifically binds podocalyxin to determine the level of podocalyxin. Aptamers suitable for use in the present disclosure will be apparent to the skilled person and/or are described herein.

In one example, the antibody or aptamer is conjugated to a detectable label. For example, the antibody is conjugated to a detectable label. In another example, the aptamer is conjugated to a detectable label. Detectable labels suitable for use in the present disclosure will be apparent to the skilled person and/or are described herein. For example, the detectable label is selected from the group consisting of a radiolabel, an enzyme, a fluorescent label, a luminescent label, a bioluminescent label, a magnetic label, a prosthetic group, a contrast agent and an ultrasound agent.

In one example, the detectable label is a radiolabel. For example, the radiolabel can be, but is not limited to, radioiodine (125I, 131I); technetium; yttrium; 35S or 3H.

In one example, the detectable label is an enzyme. For example, the enzyme can be, but is not limited to, horseradish peroxidase, alkaline phosphatase, β-galactosidase, or acetylcholinesterase.

In one example, the detectable label is a fluorescent label. For example, the fluorescent label can be, but is not limited to, umbelliferone, fluorescein, fluorescein isothiocyanate, rhodamine, dichlorotriazinylamine fluorescein, dansyl chloride or phycoerythrin.

In one example, the detectable label is a luminescent label. For example, the luminescent label can be, but is not limited to, luminol.

In one example, the detectable label is a bioluminescent label. For example, the bioluminescent label can be, but is not limited to, luciferase, luciferin or aequorin.

In one example, the detectable label is a magnetic label. For example, the magnetic label can be, but is not limited to, gadolinium or iron-oxide chelate.

In one example, the detectable label is a prosthetic group. For example, the prosthetic group can be, but is not limited to, streptavidin/biotin or avidin/biotin.

In one example, the detectable label is a contrast agent.

In one example, the detectable label is an ultrasound agent. For example, the ultrasound agent can be, but is not limited to, a microbubble-releasing agent. In one example, the ultrasound agent is a microbubble-releasing agent.

In one example, determining the level of podocalyxin comprises determining the level of a downstream regulator of progesterone and/or an upstream regulator of podocalyxin. For example, the downstream regulator of progesterone and/or an upstream regulator of podocalyxin is a microRNA. In another example, the method comprises determining the level of a microRNA to determine the level of podocalyxin. For example, the microRNA is miR-199 or miR-145. In a further example, there is an inverse relationship between the level of the microRNA and the level of podocalyxin. For example, an elevated level of the microRNA is indicative of a lower level of podocalyxin.

Methods of detecting the level of podocalyxin will be apparent to the skilled person and/or described herein. For example, the method comprises performing an immunohistochemical assay, in situ hybridization, flow cytometry, an enzyme-linked immunosorbent assay, western blot, real-time reverse transcription polymerase chain reaction (RT-PCR) or ultrasound molecular imaging.

In one example, the method comprises performing an immunohistochemical assay.

In one example, the method comprises performing flow cytometry.

In one example, the method comprises performing an enzyme-linked immunosorbent assay.

In one example, the method comprises performing western blot.

In one example, the method comprises performing real-time reverse transcription polymerase chain reaction (RT-PCR).

In one example, the method comprises performing ultrasound molecular imaging.

In one example, the method is performed on endometrial epithelial cells in vitro or ex vivo. For example, the method is performed on endometrial epithelial cells in vitro. In another example, the method is performed on endometrial epithelial cells ex vivo.

In one example, the method is performed on endometrial epithelial cells obtained from the subject in a biological sample. Suitable biological samples for use in the present disclosure will be apparent to the skilled person and/or are described herein. For example, the biological sample is selected from the group consisting of an endometrial biopsy, a uterine fluid sample and a vaginal fluid sample.

In one example, the biological sample is an endometrial biopsy.

In one example, the biological sample is endometrial epithelial cells.

In one example, the biological sample is a uterine fluid sample.

In one example, the biological sample is a vaginal fluid sample.

In one example, the subject has been previously treated with a composition comprising progesterone, progestogen or an analog or combinations thereof. For example, the subject has been receiving treatment for infertility. In another example, the subject has been receiving treatment due to embryo implantation failure.

In one example, the level of podocalyxin is determined in at least one biological sample and at least one time point during a cycle. For example, the level of podocalyxin is determined at 1, or 2, or 3, or 4, or 5, or 6, or 7, or 8, or 9, or 10 time points during a cycle.

In one example, the method further comprises implantation of an embryo into the subject. For example, implantation of the embryo is based on the level of podocalyxin in the subject.

In one example, the level of podocalyxin is determined in a first cycle of the subject and an embryo is implanted in a subsequent cycle of the subject.

The present disclosure also provides a method of detecting infertility in a subject, the method comprising determining a level of podocalyxin in endometrial epithelial cells in the subject.

The present disclosure further provides a method of diagnosis and prognosis of infertility in a subject, the method comprising determining a level of podocalyxin in endometrial epithelial cells in the subject.

In one example, the level of podocalyxin is determined in at least one biological sample and at least one time point during a cycle.

The present disclosure also provides a method of monitoring endometrial epithelial receptivity and predicting optimal endometrial epithelial receptivity for embryo implantation in a subject, the method comprising determining a level of podocalyxin in endometrial epithelial cells in the subject at one or more time points.

The present disclosure also provides a method of improving endometrial epithelial receptivity for embryo implantation in a subject, the method comprising determining a level of podocalyxin in endometrial epithelial cells in the subject, and based on the level of podocalyxin in the cells, administering to the subject a compound in an amount sufficient to reduce the level of podocalyxin in the endometrial epithelial cells.

The present disclosure further provides a method of assessing effectiveness of a compound on improving endometrial epithelial receptivity for embryo implantation in a subject, the method comprising determining a level of podocalyxin in endometrial epithelial cells in the subject, wherein the subject has previously received treatment with the compound.

The present disclosure also provides a method of optimising treatment with a compound to improve endometrial epithelial receptivity for embryo implantation in a subject, the method comprising administering to the subject a compound, determining a level of podocalyxin in endometrial epithelial cells in the subject and optionally, based on the level of podocalyxin, modifying the treatment to the subject.

In one example, the modification is one or more or all of dose, type of compound and/or route of administration.

In one example, the compound is selected from the group consisting of progesterone, progestogen, or an analog thereof, an antisense polynucleotide, a catalytic nucleic acid, an interfering RNA, a siRNA, a microRNA and combinations thereof. For example, the compound is a microRNA, such as miR-199 or miR-145.

### BRIEF DESCRIPTION OF FIGURES

FIG. 1 is a graphical representation showing real-time qRT-PCR analysis of podocalyxin (PCX) mRNA expression in HUVECs and HEECs. Data are expressed as mean±SD.

FIG. 2 is a graphical representation showing quantification of PCX immunohistochemical staining intensity in (A) luminal epithelium (LE); (B) glandular epithelium (GE), and (C) blood vessels (BV) in the proliferative (Prolif), early (E)-, mid (M)- and late (L)-secretory (Sec) phase of the menstrual cycle. Data are expressed as mean±SD. Prolif; E-Sec; M-Sec; L-Sec. *P<0.05, **P<0.005, ***P<0.0005.

FIG. 3 is a graphical representation showing the (A) mRNA levels and (B) protein levels of PCX in primary HEECs treated with estrogen (E) without or with progesterone (P) for 48, 72 and 98 h. Data are expressed as mean±SD, *P<0.05, **P<0.005.

FIG. 4 is a graphical representation showing the effect of transient knockdown (KD) or stable overexpression (PCX-OE) of PCX in Ishikawa cells. Transient knockdown of PCX reduced PCX mRNA expression (A) and increased adhesion to fibronectin (B). Overexpression of PCX increased PCX mRNA expression (C) and decreased adhesiveness to fibronectin. Mean±SD, ***P<0.0005, ****P<0.0001.

FIG. 5 is a graphical representation showing quantification of the attachment of primary trophoblast spheroids onto the PCX overexpressing Ishikawa monolayer. Mean±SD, n=3-5*P<0.05, **P<0.005, ****P<0.0001.

FIG. 6 is a graphical representation showing quantification of the invasion of primary trophoblast spheroids through the PCX overexpressing Ishikawa monolayer. Mean±SD, n=3*p<0.05, **p<0.005.

FIG. 7 is a graphical representation showing quantification of the (A) attachment and (B) invasion of human embryos onto the PCX overexpressing Ishikawa monolayer. Mean±SD, n=3, **P<0.005; *p<0.05

FIG. 8 is a graphical representation showing real-time qRT-PCR analysis of (A-F) up-regulated and (G-L) down-regulated genes between control and PCX-OE Ishikawa cells. Mean±SD, n=3. *P<0.05, **P<0.005, ***P<0.0005 ****P<0.0001.

FIG. 9 is a graphical representation showing (A) the trans-epithelial electrical resistance (TER) and (B) flux of FITC-dextran of control and PCX-OE cells. Mean±SD, n=3**P<0.005.

FIG. 10 is a graphical representation showing the proportions of implantation success and failure in PCX− and PCX+ groups *P=0.036, Fisher's exact test.

FIG. 11 is a graphical representation showing real-time RT-PCR analysis of mir145 and mir199 in primary endometrial epithelial cells following E+P vs E treatment. Fold change ±SD in E+P cells relative to E cells, n=4, *P<0.05.

FIG. 12 is a graphical representation showing real-time RT-PCR analysis of PCX mRNA in Ishikawa cells following transfection with mir145, mir199 or their combination. Fold change ±SD relative to control cells at 24 h, n=4.

### KEY TO SEQUENCE LISTING

SEQ ID NO: 1 PODXL (PCX) forward primer

SEQ ID NO: 2 PODXL (PCX) reverse primer

SEQ ID NO: 3 CDH1 forward primer

SEQ ID NO: 4 CDH1 reverse primer

SEQ ID NO: 5 TJP1 forward primer

SEQ ID NO: 6 TJP1 reverse primer

SEQ ID NO: 7 CLDN4 forward primer

SEQ ID NO: 8 CLDN4 reverse primer

SEQ ID NO: 9 OCLN forward primer

SEQ ID NO: 10 OCLN reverse primer

SEQ ID NO: 11 WNT7A forward primer

SEQ ID NO: 12 WNT7A reverse primer

SEQ ID NO: 13 LEFTY2 forward primer

SEQ ID NO: 14 LEFTY2 reverse primer

SEQ ID NO: 15 LIF forward primer

SEQ ID NO: 16 LIF reverse primer

SEQ ID NO: 17 CSF1 forward primer

SEQ ID NO: 18 CSF1 reverse primer

SEQ ID NO: 19 ERBB4 forward primer

SEQ ID NO: 20 ERBB4 reverse primer

SEQ ID NO: 21 FGF2 forward primer

SEQ ID NO: 22 FGF2 reverse primer

SEQ ID NO: 23 TGFB1 forward primer

SEQ ID NO: 24 TGFB1 reverse primer

SEQ ID NO: 25 MMP14 forward primer

SEQ ID NO: 26 MMP14 reverse primer

SEQ ID NO: 27 YWHAZ forward primer

SEQ ID NO: 28 YWHAZ reverse primer

SEQ ID NO: 29 18S forward primer

SEQ ID NO: 30 18S reverse primer

SEQ ID NO: 31 hsa-miR-199a-5p

SEQ ID NO: 32 hsa-miR-152-3p

SEQ ID NO: 33 hsa-miR-145-5p

SEQ ID NO: 34 hsa-miR-219a-5p

SEQ ID NO: 35 hsa-miR-34a-5p

SEQ ID NO: 36 hsa-mir-181a-5p

SEQ ID NO: 37 hsa-miR-144-3p

SEQ ID NO: 38 hsa-miR-802

SEQ ID NO: 39 hsa-miR-125b-5p

SEQ ID NO: 40 hsa-miR-143-3p

SEQ ID NO: 41 hsa-miR-202-5p

SEQ ID NO: 42 hsa-miR-506-3p (124-3p.2)

SEQ ID NO: 43 hsa-miR-16-5p (15-5p)

SEQ ID NO: 44 hsa-miR-361-5p (Control)

### DETAILED DESCRIPTION OF THE INVENTION

### General Definitions

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

The present disclosure is not to be limited in scope by the specific examples described herein, which are intended for the purpose of exemplification only. Functionally-equivalent products, compositions and methods are clearly within the scope of the present disclosure.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, reproductive biology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, Perbal 1984; Sambrook 1989; Brown 1991; Glover 1995; Ausubel 1988; Harlow 1988; Coligan 1991.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” shall be taken to mean any animal including humans, for example a mammal. Exemplary subjects include but are not limited to humans and non-human primates. For example, the subject is a human. In one example, the subject is a female human.

### Endometrial Epithelial Receptivity

Endometrial remodelling is a key feature of the human menstrual cycle and the conversion from a non-adhesive to an adhesive state is critical for embryo implantation. In particular, the apical surface of the luminal epithelium, which directly interacts with the implanting embryo to initiate attachment, must remodel for receptivity. It is therefore desirable to be able to determine the optimal point during the cycle when the endometrium is receptive to embryo implantation.

It will be apparent to the skilled person that the present disclosure provides methods for determining the optimal timing for a naturally achieved pregnancy, for example implantation following naturally achieved conception, or a pregnancy achieved with an assisted reproductive technology.

The present inventors have found that endometrial epithelial cells intrinsically express podocalyxin as a key anti-implantation regulator, which must be down-regulated in the epithelium for receptivity. Specifically the inventors have surprisingly found that down-regulation of the regulator in the endometrial luminal epithelium and not the glandular epithelium signifies endometrial epithelial receptivity.

As used herein, the term “endometrial epithelial receptivity” refers to a time period of the menstrual cycle during which the endometrium is receptive to implantation. During this period, the endometrium acquires a functional state allowing adhesion of the blastocyst. This period preferably corresponds to the mid-secretory phase of the menstrual cycle or days 20 to 24 of a 28 day menstrual cycle in humans.

The inventors have also demonstrated that up-regulation or elevated levels of the podocalyxin in both the luminal and glandular cells of the endometrial epithelium signals pre-receptivity.

As used herein, the term “pre-receptivity” or “pre-endometrial epithelial receptivity” refers to a time period of the menstrual cycle during which the endometrium is not yet receptive to implantation however is in the process of becoming receptive to implantation in that cycle.

The inventors have also demonstrated that downregulation or reduced levels of podocalyxin in both the luminal and glandular cells of the endometrial epithelium signals post-receptivity.

As used herein, the term “post-receptivity” or “post-endometrial epithelial receptivity” refers to a time period of the menstrual cycle during which the endometrium has been receptive to implantation however, the time period during that cycle for implantation has occurred.

As used herein, the term “cycle” or “menstrual cycle” refers to the process of ovulation and menstruation in women and other female primates. The skilled person would understand that this term encompasses the changes associated with both the ovaries (also known as the ovarian cycle) and the lining of the uterus or endometrium (also known as the uterine cycle). The ovarian cycle consists of the follicular phase, ovulation and the luteal phase, and the uterine cycle consists of menstruation, the proliferative phase and the secretory phase. The average menstrual cycle in humans is 28 days.

In one example, the present disclosure provides a method of predicting endometrial epithelial receptivity in a subject in need thereof.

### Determining the Level of Podocalyxin

Podocalyxin (PODXL or PCX), also known as podocalyxin-like protein 1 (PCLP-1), is a member of the CD34 family of transmembrane sialomucins and is implicated in the regulation of cell adhesion, migration and polarity. PODXL is expressed by kidney podocytes, hematopoietic progenitors, vascular endothelia, and a subset of neurons; whilst aberrant expression has been implicated in a range of cancers. As a type I transmembrane protein, PODXL has an extensively O-glycosylated and sialylated extracellular domain and transmembrane region and a short intracellular region. The encoded protein has a 22 amino acid signal peptide, an extracellular domain of 439 residues, a 21 residue transmembrane domain and a 76 amino acid C-terminal intracellular domain. For the purposes of nomenclature only and not limitation an exemplary sequence of human PODXL is set out in NCBI Reference Sequence NG_042104.1. It should be understood that the term ‘Podocalyxin (PODXL or PCX)’ includes any isoform which may arise from alternative slicing of podocalyxin mRNA or mutant or polymorphic forms of podocalyxin. For example, for the purposes of nomenclature only and not limitation exemplary sequences of human PODXL isoforms 1 and 2 are set out in GenBank Accession no. NP_001018121 and GenBank Accession no. NP_005388, respectively. The sequence of PODXL from other species can be determined using sequences provided herein and/or in publicly available databases and/or determined using standard techniques (e.g., as described in Ausubel 1988 or Sambrook 1989).

The present inventors have found that podocalyxin is down regulated markedly in luminal epithelial cells at the time of receptivity establishment.

Accordingly, the methods of any disclosure described herein comprise determining a level of podocalyxin in endometrial epithelial cells in the subject.

As used herein, the term “level” in reference to podocalyxin shall be understood to refer to the level of functionality of the gene and/or protein (i.e., the functional level). For example, the level (or “level of expression”) refers to a measure of the mRNA transcript expressed by the gene or a measure of the encoded protein.

In one example, determining the level of podocalyxin comprises determining the amount of podocalyxin protein, and/or determining the amount of nucleic acid molecules encoding podocalyxin, in the endometrial epithelial cells.

As used herein, the term “amount” with reference to the level of podocalyxin will be understood to refer to a quantity of mRNA molecules and/or protein. Various methods of assessing the distribution pattern are available to the skilled person and the skilled person will recognise that the specific value or amount will vary depending on the method of assessment used. It will also be apparent that this term encompasses both an absolute and relative value. For example, the amount may be relative to a reference or control sample, the number of cells assessed (e.g., amount per 100 cells) and/or the type of cells (e.g., luminal versus glandular epithelial cells). In another example, the amount may be an absolute value of the amount of mRNA molecules and/or protein present in the sample.

In one example, determining the level of podocalyxin comprises determining the distribution pattern of podocalyxin protein.

As used herein, the term “distribution pattern” refers to the specific pattern and/or cellular localisation of podocalyxin protein in the subject. Various methods of assessing the distribution pattern are available to the skilled person and will be dependent on the method of analysis used. The skilled person will recognise that this term encompasses descriptive analyses (e.g., presence or absence), multiparametric and semi-quantitative scoring (e.g., strong, weak or absent).

In one example, the level of podocalyxin is the level in a population of cells.

Reference to a “population of cells” or “cell population” in the present disclosure refers to all endometrial epithelial cells. It will be apparent to the skilled person that the endometrium is comprised of both luminal and glandular epithelial cells and that the term encompasses both populations of cells.

As used herein, the term “luminal epithelium” (LE) refers to the cells that line the lumen of the uterus.

The term “glandular epithelium” (GE) as used herein refers to the cells of the endometrial or uterine glands.

Accordingly, it will be apparent to the skilled person that the level of podocalyxin in a subject may be the level in the population of cells (i.e., in both the glandular and luminal epithelial cells), or the level of podocalyxin may be the level in a subset of the population of cells (i.e., in either the glandular or luminal epithelial cells).

In one example, the level of podocalyxin is the level of podocalyxin in the luminal and glandular epithelial cells. For example, the level of podocalyxin is compared to a reference or control.

In one example, the level of podocalyxin is the level of podocalyxin in the luminal or glandular epithelial cells. For example, the level of podocalyxin is the level of podocalyxin in the luminal epithelial cells. In another example, the level of podocalyxin is the level of podocalyxin in the glandular epithelial cells. In one example, the level of podocalyxin in the luminal or glandular epithelial cells is compared to a reference or control. In another example, the level of podocalyxin in the luminal epithelial cells is compared to the level of podocalyxin in the glandular epithelial cells. In another example, the level of podocalyxin in the glandular epithelial cells is compared to the level of podocalyxin in the luminal epithelial cells.

In one example of any method described herein, the method comprises determining (a) if the level of the podocalyxin in the subject is higher than the level of the podocalyxin in the reference, or (b) if the level of the podocalyxin in the subject is lower than the level of podocalyxin in the reference.

The term “higher” in reference to the level of podocalyxin means that the level of nucleic acid molecule encoding podocalyxin or podocalyxin protein in the subject is greater or increased, compared to a control or reference level, or in one cell population compared to another. It will be apparent from the foregoing that the level of podocalyxin needs only be increased by a statistically significant amount, for example, by at least about 10%, or about 20%, or about 30%, or about 40%, or about 50%, or about 60%, or about 70%, or about 80%, or about 90%, or about 95%.

The term “lower” in reference to the level of podocalyxin expression means that the level of nucleic acid molecule encoding podocalyxin or podocalyxin protein in the subject is reduced or decreased, compared to a control or reference level, or in one cell population compared to another. It will be apparent from the foregoing that the level of podocalyxin need only be decreased by a statistically significant amount, for example, by at least about 10%, or about 20%, or about 30%, or about 40%, or about 50%, or about 60%, or about 70%, or about 80%, or about 90%, or about 95%.

### Methods of Determining the Level of Podocalyxin

Methods of determining the level of podocalyxin nucleic acid molecules encoding podocalyxin or podocalyxin protein will be apparent to the skilled person and/or are described herein.

### Determining the Level of Nucleic Acid Molecules

Methods for detecting nucleic acids are known in the art and include, for example, hybridization-based assays, amplification-based assays and restriction endonuclease-based assays. For example, levels of a transcribed gene can be determined by polymerase chain reaction (PCR) amplification, ligase chain reaction or cycling probe technology amongst others.

### Primer Design and Production

As will be apparent to the skilled person, the specific primer used in an assay of the present disclosure will depend upon the assay format used. Clearly, a primer that is capable of specifically hybridizing to or detecting a marker of interest is preferred. Methods for designing primers for, for example, PCR or hybridization are known in the art and described, for example, in Dieffenbach 1995. Furthermore, several software packages are publicly available that design optimal primers for a variety of assays, e.g. Primer 3 available from the Center for Genome Research, Cambridge, Mass., USA. Primers suitable for use in the present disclosure are preferably those that do not form hairpins, self-prime or form primer dimers (e.g. with another primer used in a detection assay).

Furthermore, a primer (or the sequence thereof) is assessed to determine the temperature at which it denatures from a target nucleic acid (i.e. the melting temperature of the probe or primer, or Tm). Methods of determining Tm are known in the art and described, for example, in Santa Lucia, 1995 or Bresslauer et al., 1986.

Exemplary primers used for the detection of podocalyxin in the present disclosure include:

hPODXL-Forward: 5′-GAGCAGTCAAAGCCACCTTC-3′,
hPODXL-Reverse: 5′-TGGTCCCCTAGCTTCATGTC-3′;

Suitable control primers will also be apparent to the skilled person and include, for example, 18 s and β-Actin. Exemplary control sequences for use in the present disclosure include:

18s-Forward: 5′-CGGCTACCACATCCAAGGAA-3′
18s-Reverse: 5′-GCTGGAATTACCGCGGCT-3′

Methods for producing/synthesizing a primer of the present disclosure are known in the art. For example, oligonucleotide synthesis is described, in Gait 1984. For example, a probe or primer may be obtained by biological synthesis (e.g. by digestion of a nucleic acid with a restriction endonuclease) or by chemical synthesis. For short sequences (up to about 100 nucleotides) chemical synthesis is preferable.

In one example, the primer comprises one or more detectable markers. For example, the primer comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, the primer is labeled with, for example, a fluorescent semiconductor nanocrystal (as described, for example, in U.S. Pat. No. 6,306,610), a radiolabel or an enzyme (e.g. horseradish peroxidase (HRP), alkaline phosphatase (AP) or β-galactosidase).

Such detectable labels facilitate the detection of a primer, for example, the hybridization of the primer or an amplification product produced using the primer. Methods for producing such a labeled primer are known in the art. Furthermore, commercial sources for the production of a labeled primer are known to the skilled artisan, e.g., Sigma-Genosys, Sydney, Australia.

### Polymerase-Chain Reaction (PCR)

Methods of PCR are known in the art and described, for example, in Dieffenbach 1995. Generally, for PCR two non-complementary nucleic acid primer molecules comprising at least about 20 nucleotides or at least about 30 nucleotides are hybridized to different strands of a nucleic acid template molecule, and specific nucleic acid molecule copies of the template are amplified enzymatically. PCR products may be detected using electrophoresis and detection with a detectable marker that binds nucleic acids. Alternatively, one or more of the oligonucleotides are labeled with a detectable marker (e.g., a fluorophore) and the amplification product detected using, for example, a lightcycler (Perkin Elmer, Wellesley, Mass., USA). Alternatively, PCR products are detected, for example, using mass spectrometry. Clearly, the present disclosure also encompasses quantitative forms of PCR (such as real-time PCR; RT-PCR), such as, for example, a TaqMan assay. The TaqMan assay (as described in U.S. Pat. No. 5,962,233) uses allele specific (ASO) probes with a donor dye on one end and an acceptor dye on the other end such that the dye pair interact via fluorescence resonance energy transfer (FRET).

### Ligase Chain Reaction (LCR)

Ligase chain reaction (described in, for example, EU 320,308 and U.S. Pat. No. 4,883,750) uses two or more oligonucleotides that hybridize to adjacent target nucleic acids. A ligase enzyme is then used to link the oligonucleotides. In the presence of one or more nucleotide(s) that is(are) not complementary to the nucleotide at an end of one of the primers that is adjacent to the other primer, the ligase is unable to link the primers, thereby failing to produce a detectable amplification product. Using thermocycling the ligated oligonucleotides then become a target for further oligonucleotides. The ligated fragments are then detected, for example, using electrophoresis, or MALDI-TOF. Alternatively, or in addition, one or more of the probes is labeled with a detectable marker, thereby facilitating rapid detection.

### Cycling Probe Technology

Cycling Probe Technology uses chimeric synthetic probe that comprises DNA-RNA-DNA that is capable of hybridizing to a target sequence. Upon hybridization to a target sequence the RNA-DNA duplex formed is a target for RNase H that cleaves the probe. The cleaved probe is then detected using, for example, electrophoresis or MALDI-TOF.

### Qβ Replicase

Qβ Replicase, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

### Strand Displacement Amplification (SDA)

Strand displacement amplification (SDA) utilizes oligonucleotides, a DNA polymerase and a restriction endonuclease to amplify a target sequence. The oligonucleotides are hybridized to a target nucleic acid and the polymerase used to produce a copy of this region. The duplexes of copied nucleic acid and target nucleic acid are then nicked with an endonuclease that specifically recognizes a sequence of nucleotides at the beginning of the copied nucleic acid. The DNA polymerase recognizes the nicked DNA and produces another copy of the target region at the same time displacing the previously generated nucleic acid. The advantage of SDA is that it occurs in an isothermal format, thereby facilitating high-throughput automated analysis.

### Other Nucleic Acid Amplification Methods

Other nucleic acid amplification procedures include transcription-based amplification systems (TAS), including nucleic acid sequence based amplification (NASBA) and 3SR (WO 88/10315).

Methods for direct sequencing of nucleotide sequences are well known to those skilled in the art and can be found for example in Ausubel 1995 and Sambrook 1989. Sequencing can be carried out by any suitable method, for example, dideoxy sequencing, chemical sequencing, next generation sequencing techniques or variations thereof. Direct sequencing has the advantage of determining variation in any base pair of a particular sequence.

### Determining the Level of Podocalyxin Polypeptide or Protein

Methods for detecting the amount or level of podocalyxin protein or polypeptide (including different isoforms) are known in the art and include, for example, immunohistochemistry, immunofluorescence, an immunoblot, a western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fibre-optics technology or protein chip technology. For example, a suitable assay is a semi-quantitative assay and/or a quantitative assay.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

In one example, the method for determining the level of podocalyxin in a sample comprises contacting a biological sample from a subject with an antibody or ligand that specifically binds to the podocalyxin polypeptide or protein for a time and under conditions sufficient for a complex between the antibody or ligand and the polypeptide or protein to form and then detecting the complex.

### Ligands

As used herein the term “ligand” shall be taken to include any compound, molecule, peptide, polypeptide, protein, nucleic acid, chemical, small molecule, natural compound, etc that is capable of specifically binding to a podocalyxin polypeptide. Such a ligand may bind to a podocalyxin polypeptide by any process, for example, by hydrogen bonding, a van der Waals interaction, a hydrophobic interaction, an electrostatic interaction, disulphide bond formation or covalent bond formation.

### Antibodies

As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)2, and Fv fragments.

Antibodies suitable for use in the detection of podocalyxin will be apparent to the skilled person and/or described herein and include, for example, commercially available antibodies AF1658 (R&D Systems); 3D3 (Santa Cruz) and/or EPR9518 (Abcam).

In one example, the antibody specifically binds podocalyxin to determine the level of podocalyxin.

As used herein, the term “specifically binds” or “binds specifically” shall be taken to mean that an antibody reacts or associates more frequently, more rapidly, with greater duration and/or with greater affinity with a particular antigen or cell expressing same than it does with alternative antigens or cells. Generally, but not necessarily, reference to binding means specific binding, and each term shall be understood to provide explicit support for the other term.

Antibodies may be prepared by any of a variety of techniques known to those of ordinary skill in the art, and described, for example in, Harlow 1988. In one such technique, an immunogen comprising a podocalyxin polypeptide or a fragment thereof is injected into any one of a variety of mammals (e.g., mice, rats, rabbits, sheep, pigs, chickens or goats). The immunogen is derived from a natural source, produced by recombinant expression means, or artificially generated, such as by chemical synthesis (e.g., BOC chemistry or FMOC chemistry). In this method, a podocalyxin polypeptide or a fragment thereof may serve as the immunogen without modification. Alternatively, a podocalyxin polypeptide or a fragment thereof is joined to a carrier protein, such as, for example bovine serum albumin. The immunogen and optionally a carrier for the protein is injected into the animal host, preferably according to a predetermined schedule incorporating one or more booster immunizations, and blood collected from the said animals periodically. Optionally, the immunogen is injected in the presence of an adjuvant, such as, for example, Freund's complete or incomplete adjuvant to enhance the immune response to the immunogen.

Monoclonal antibodies specific for the antigenic polypeptide of interest may be prepared, for example, using the technique of Kohler et al., 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described supra. The spleen cells are immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, and thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification as described supra. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

Alternatively, a monoclonal antibody capable of binding to a form of a podocalyxin polypeptide of interest or a fragment thereof is produced using a method such as, for example, a human B-cell hybridoma technique (Kozbar et al., 1983), a EBV-hybridoma technique to produce human monoclonal antibodies (Cole 1985), or screening of combinatorial antibody libraries (Huse et al., 1989).

In one example, the antibody is conjugated to a detectable label.

As used herein, a “detectable label” is a molecular or atomic tag or marker that generates or can be induced to generate an optical or other signal or product that can be detected visually or by using a suitable detector. Detectable labels are well known in the art and include, for example, a radiolabel, an enzyme, a fluorescent label, a luminescent label, a bioluminescent label, a magnetic label, a prosthetic group, a contrast agent and an ultrasound agent.

Fluorescent labels commonly used include Alexa, cyanine such as Cy5 and Cy5.5, and indocyanine, and fluorescein isothiocyanate (FITC), but they are not so limited. Fluorescent labels useful in the practice of the present disclosure can include, also without limitation, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6C; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange+DNA; Acridine Orange+RNA; Acridine Orange, both DNA & RNA; Acridine Red; Acridine Yellow; Acriflavin; Acriflavin Feulgen SITSA; Aequorin (Photoprotein); Alexa Fluor 350; Alexa Fluor 430; Alexa Fluor 488; Alexa Fluor 532; Alexa Fluor 546; Alexa Fluor 568; Alexa Fluor 594; Alexa Fluor 633; Alexa Fluor 647; Alexa Fluor 660; Alexa Fluor 680; Alizarin Complexon; Alizarin Red; Allophycocyanin (APC); AMC, AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTRA-BTC=Ratio Dye, Zn²⁺; APTS; Astrazon Brilliant Red 4G; Astrazon Orange R; Astrazon Red 6B; Astrazon Yellow 7 GLL; Atabrine; ATTO-TAG CBQCA; ATTO-TAG FQ; Auramine; Aurophosphine G; Aurophosphine; BAO 9 (Bisamninophenyloxadiazole); BCECF (high pH); BCECF (low pH); Berberine Sulphate; Beta Lactamase; BFP blue shifted GFP (Y66H); Blue Fluorescent Protein; BFP/GFP FRET Bimane; Bisbenzamnide; Bisbenzimide (Hoechst); bis-BTC=Ratio Dye, Zn²⁺; Blancophor FFG; Blancophor SV; BOBO-1; BOBO-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO-1; BO-PRO-3; Brilliant Sulphoflavin FF; BTC-Ratio Dye Ca²⁺; BTC-5N-atio Dye, Zn²⁺; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue; Cascade Yellow 399; Catecholamine; CCF2 (GeneBlazer); CFDA; CFP-Cyan Fluorescent Protein; CFP/YFP; FRET; Chlorophyll; Chromomycin A; Chromomycin A; CL-NERF (Ratio Dye, pH); CMFDA; Coelenterazine; Coelenterazine cp (Ca²⁺ Dye); Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2; Cy3.1 8; Cy3.5; Cy3; Cy5.1 8; Cy5.5; Cy5; Cy7; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; Dopamine; DsRed; Red fluorescent protein; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyde Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43; FM 4-46; Fura Red (high pH); Fura Red/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP), GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; LIVE/DEAD Kit Animal Cells, Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue, LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green; Oregon Green 488-X; Oregon Green; Oregon Green 488; Oregon Green 500; Oregon Greene 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodide (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP; sgBFP (super glow BFP); sgGFP; sgGFP (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYT; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red; Texas Red-X conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamine-IsoThioCyanate); True Blue; TruRed; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO-3; YOYO-1; and YOYO-3.

In one example, a detectable label is an enzyme. The enzyme can act on an appropriate substrate to result in production of a detectable dye. Examples of enzymes useful in the disclosure include, without limitation, alkaline phosphatase and horseradish peroxidase. Alternatively or in addition, the enzyme can be, for example, luciferase. The enzyme can be linked to the antibody by conventional chemical methods, or it can be expressed together with the antibody as a fusion protein.

Radioisotopes useful as detectable labels in the disclosure are well known in the art and can include ³H, ¹¹C, ¹⁸F, ³⁵S, ⁶⁴Cu, ⁶⁷Ga, ⁶⁸Ga, ⁹⁹mTc, ¹¹¹In, ¹²³I, ¹²⁴I ¹²⁵I, and ¹³¹I. Attachment of any gamma emitting radioactive materials, e.g., ⁹⁹mTc and ¹¹¹In, which can react with carboxyl, amino, or sulfhydryl groups of a compound that binds calcitonin receptor is suitable for use in detection methods using gamma scintigraphy. Attachment of radioactive ¹¹C, ¹⁸F, ⁶⁴Cu, ⁶⁷Ga, ⁶⁸Ga, ¹²⁴I, and ¹³¹I compounds which can react with carboxyl, amino, or sulfhydryl groups of a compound is suitable for use in detection methods using PET/SPECT imaging.

### Enzyme Linked Immunosorbent Assay (ELISA) and Fluorescence Linked Immunosorbent Assay (FLISA)

Standard solid-phase ELISA or FLISA formats are particularly useful in determining the concentration of a protein from a variety of samples. In one form such an assay involves immobilizing a biological sample onto a solid matrix, such as, for example a polystyrene or polycarbonate microwell or dipstick, a membrane, or a glass support (e.g. a glass slide).

An antibody that specifically binds to a marker within a podocalyxin polypeptide is brought into direct contact with the immobilized biological sample, and forms a direct bond with any of its target protein present in said sample. This antibody is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a second labeled antibody can be used that binds to the first antibody. Following washing to remove any unbound antibody the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label.

Such ELISA or FLISA based systems are suitable for quantification of the amount of a protein in a sample, by calibrating the detection system against known amounts of a protein standard to which the antibody binds, such as for example, an isolated and/or recombinant podocalyxin polypeptide or immunogenic fragment thereof or epitope thereof.

In another example, an ELISA consists of immobilizing an antibody or ligand that specifically binds a marker of a disease or disorder within a podocalyxin polypeptide on a solid matrix, such as, for example, a membrane, a polystyrene or polycarbonate microwell, a polystyrene or polycarbonate dipstick or a glass support. A sample is then brought into physical relation with said antibody, and said marker within the sample is bound or ‘captured’. The bound protein is then detected using a labeled antibody. Alternatively, a third labeled antibody can be used that binds the second (detecting) antibody.

It will be apparent to the skilled person that the assay formats described herein are amenable to high throughput formats, such as, for example automation of screening processes or a microarray format as described in Mendoza et al., 1999. Furthermore, variations of the above-described assay will be apparent to those skilled in the art, such as, for example, a competitive ELISA.

### Western Blotting

In another example, western blotting is used to determine the level of a marker within a podocalyxin polypeptide in a sample. In such an assay protein from a sample is separated using sodium dodecyl sulphate polyacrylamide gel electrophoresis (SDS-PAGE) using techniques known in the art and described in, for example, Scopes 1994. Separated proteins are then transferred to a solid support, such as, for example, a membrane (e.g., a PVDF membrane), using methods known in the art, for example, electrotransfer. This membrane is then blocked and probed with a labeled antibody or ligand that specifically binds to a marker within a podocalyxin polypeptide. Alternatively, a labeled secondary, or even tertiary, antibody or ligand is used to detect the binding of a specific primary antibody. The level of label is then determined using an assay appropriate for the label used.

An appropriate assay will be apparent to the skilled artisan and include, for example, densitometry. In one example, the intensity of a protein band or spot is normalized against the total amount of protein loaded on a SDS-PAGE gel using methods known in the art. Alternatively, the level of the marker detected is normalized against the level of a control/reference protein. Such control proteins are known in the art, and include, for example, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP).

### Immunohistochemistry

As will be apparent to the skilled person a histochemical method, such as, for example immunohistochemistry and/or immunofluorescence as described herein, is useful for determining/detecting the subcellular localization of podocalyxin. Such methods are known in the art and described, for example, in Immunohistochemistry (Cuello 1984).

Methods of analysing localisation of podocalyxin in histochemical methods will be apparent to the skilled person and/or described herein. Exemplary methods include, for example:
Evaluation of positively stained cells and structures. For example, the cells and/or structures considered positive are counted to determine an absolute quantity of positively stained cells for each sample.
Evaluation of positively stained cells and/or area ratio. For example, the percentage of positively stained cells is determined and is relative to the total number of cells counted and/or the total area assessed. A combination of quantitative and qualitative scoring may be used when a percentage is given a certain score value. For example, a “presence” score is given for ≥66% of positive stained cells; an “absence” score is given when less, than 10% of cells or no visible staining is observed. In another example, samples are assigned a score of 0 (no staining), 1 (<10% of cells staining), 2 (10%-50% of cells staining), or 3 (>50% of cells staining).
Qualitative scoring. For example, the force of IHC expression may be assigned to a category being either positive or negative; or negative (−), weak (+), moderate (++) and strong (+++). If the categories are signed with a numeric value instead of signs, then this approach transforms from qualitative to semi-quantitative.
Digital analysis. For example, image analysis software (e.g., Fiji 1.51o) is used to determine the mean staining (or peak pixel) intensity.

### Radioimmunoassay

Alternatively, the level is detected using a radioimmunoassay (RIA). The basic principle of the assay is the use of a radiolabeled antibody or antigen to detect antibody-antigen interactions. An antibody or ligand that specifically binds to the marker within a podocalyxin polypeptide is bound to a solid support and a sample brought into direct contact with said antibody. To detect the level of bound antigen, an isolated and/or recombinant form of the antigen is radiolabeled and brought into contact with the same antibody. Following washing, the level of bound radioactivity is detected. As any antigen in the biological sample inhibits binding of the radiolabeled antigen the level of radioactivity detected is inversely proportional to the level of antigen in the sample. Such an assay may be quantitated by using a standard curve using increasing known concentrations of the isolated antigen.

As will be apparent to the skilled person, such an assay may be modified to use any reporter molecule, such as, for example, an enzyme or a fluorescent molecule, in place of a radioactive label.

### Biosensor or Optical Immunosensor System

Alternatively, the level of a podocalyxin in a sample is determined using a biosensor or optical immunosensor system. In general, an optical biosensor is a device that uses optical principles to quantitatively convert the binding of a ligand or antibody to a target polypeptide into electrical signals. These systems can be grouped into four major categories: reflection techniques; surface plasmon resonance; fibre optic techniques and integrated optic devices. Reflection techniques include ellipsometry, multiple integral reflection spectroscopy, and fluorescent capillary fill devices. Fibre-optic techniques include evanescent field fluorescence, optical fibre capillary tube, and fibre optic fluorescence sensors. Integrated optic devices include planer evanescent field fluorescence, input grading coupler immunosensor, Mach-Zehnder interferometer, Hartman interferometer and difference interferometer sensors. These examples of optical immunosensors are described in general by Robins, 1991. More specific description of these devices are found for example in U.S. Pat. Nos. 4,810,658; 4,978,503; 5,186,897; and Brady et al., 1987.

### Biological Samples

As will be apparent to the skilled person, the type and size of the biological sample will depend upon the detection means used. For example, an assay, such as, for example, PCR may be performed on a sample comprising a single cell, although a population of cells are preferred. Furthermore, protein-based assays require sufficient cells to provide sufficient protein for an antigen based assay.

As used herein, the term “sample” or “biological sample” refers to any type of suitable material obtained from the subject. The term encompasses a clinical sample, biological fluid (e.g., cervical fluid, vaginal fluid), tissue samples, live cells and also includes cells in culture, cell supernatants, cell lysates derived therefrom. The sample can be used as obtained directly from the source or following at least one-step of (partial) purification. It will be apparent to the skilled person that the sample can be prepared in any medium which does not interfere with the method of the disclosure. Typically, the sample comprises cells or tissues and/or is an aqueous solution or biological fluid comprising cells or tissues. The skilled person will be aware of selection and pre-treatment methods. Pre-treatment may involve, for example, diluting viscous fluids. Treatment of a sample may involve filtration, distillation, separation, concentration.

In one example, the biological sample has been derived previously from the subject. Accordingly, in one example, a method as described herein according to any embodiment additionally comprises providing the biological sample.

In one example, a method as described herein according to any embodiment is performed using an extract from a sample, such as, for example, genomic DNA, mRNA, cDNA or protein.

In one example, the biological sample comprises luminal epithelial cells and/or glandular epithelial cells. For example, the biological sample comprises luminal epithelial cells. In another example, the biological sample comprises glandular epithelial cells.

### Reference Samples

As will be apparent from the preceding description, some assays of the present disclosure may utilize a suitable reference sample or control for quantification.

Suitable reference samples for use in the methods of the present disclosure will be apparent to the skilled person and/or described herein. For example, the reference may be an internal reference (i.e., from the same subject), from a normal individual or an established data set (e.g., matched by age, sample type and/or stage of cycle).

In one example, the reference is an internal reference or sample. For example, the reference is an autologous reference. In one example, the internal reference is obtained from the subject at the same time as the sample under analysis. In another example, the internal reference is obtained from the subject at an earlier time point as the sample under analysis. For example, the sample is obtained from a previous cycle.

As used herein, the term “normal individual” shall be taken to mean that the subject is selected on the basis that they are not infertile and/or are not currently pregnant.

In one example, the reference is an established data set. Established data sets suitable for use in the present disclosure will be apparent to the skilled person and include, for example:
A data set comprising endometrial epithelial cells from another subject or a population of subjects matched by age, sample type and/or stage of cycle;
A data set comprising endometrial epithelial cells in vitro, wherein the cells have been treated to induce podocalyxin expression; and
A data set comprising endometrial epithelial cells in vitro, wherein the cells have been treated to inhibit podocalyxin expression.

It will be apparent to the skilled person that the term ‘endometrial epithelial cells’ in the context of a reference sample includes glandular and/or luminal cells. For example, the reference sample comprises glandular and luminal cells. In another example, the reference sample comprises glandular cells. In a further example, the reference sample comprises luminal cells.

In one example, a reference is not included in an assay. Instead, a suitable reference is derived from an established data set previously generated. Data derived from processing, analyzing and/or assaying a test sample is then compared to data obtained for the sample.

### Monitoring Endometrial Epithelial Receptivity

It will be apparent to the skilled person that the present disclosure also provides a method of monitoring endometrial epithelial receptivity and predicting optimal endometrial epithelial receptivity for embryo implantation in a subject, the method comprising determining a level of podocalyxin in endometrial epithelial cells in the subject at one or more time points.

As used herein, the term “monitoring” in regards endometrial epithelial receptivity can include, determination of prognosis, selection of drug therapy, assessment of ongoing drug therapy, prediction of outcomes, determining response to therapy (including diagnosis of a complication), following progression of a cycle, providing information relating to a patient's menstrual cycle over time, or selecting patients most likely to benefit from therapy.

The term “optimal” as used herein refers to the most favourable period in the menstrual cycle for embryo implantation.

In one example, the method of monitoring endometrial epithelial receptivity in the subject comprises determining the level of podocalyxin at multiple time points during the cycle. For example, the level of podocalyxin is determined at a time point during the ovarian cycle and/or at a time point during the uterine cycle. In one example, the level of podocalyxin is determined during the follicular phase, ovulation and/or the luteal phase. In a further example, the level of podocalyxin is determined during menstruation, the proliferative phase and/or the secretory phase. Furthermore, the level of podocalyxin may be determined at multiple time points in a single phase of a cycle. For example, the level of podocalyxin is determined at multiple points during the secretory phase of the uterine cycle.

As discussed above, the skilled person would understand that the average menstrual cycle in humans is 28 days, however this is variable.

For example, the average duration of each of the phases of the ovarian cycle are:
Follicular phase: days 1 to 14;
Luteal Phase: days 15 to 28.

For example, the average duration of each of the phases of the uterine cycle are:
Menstruation: days 1 to 4;
Proliferative phase: days 5 to 14;
Secretory Phase: days 15 to 28.

In one example, the level of podocalyxin is compared to a level of podocalyxin in the subject at an earlier time point. Reference to an “earlier time point” in the context of the present disclosure refers to a level determined in another sample of the subject at any prior time point. For example, the earlier time point may refer to a time point in the same cycle as the sample under analysis or to the same time point in a previous cycle.

As will be apparent to the skilled person, the ability to monitor the level of podocalyxin in a subject over the duration of the cycle and/or multiple cycles will assist in predicting optimal endometrial epithelial receptivity for embryo implantation. For example, monitoring the level of podocalyxin is determined in a first cycle of the subject and an embryo is implanted in a second cycle of the subject.

### Diagnosis and Prognosis of Infertility

As disclosed herein, the inventors of the present disclosure have demonstrated a role of podocalyxin in endometrial epithelial receptivity. It will be apparent to the skilled person that the methods disclosed herein will be useful in identifying the underlying causes of infertility and implantation failure. For example, the methods of the present disclosure are useful as a screening test for the diagnosis and prognosis of infertility in a subject.

Accordingly, the present disclosure provides, for example, a method of detecting infertility in a subject, the method comprising determining a level of podocalyxin in endometrial epithelial cells in the subject.

The term “infertility” as used herein refers to a disease of the reproductive system defined by the failure to achieve a clinical pregnancy after 12 months or more of regular unprotected sexual intercourse.

The present disclosure also provides a method of diagnosis and prognosis of infertility in a subject, the method comprising determining a level of podocalyxin in endometrial epithelial cells in the subject.

As used herein, the term “diagnosis” refers to the identification of infertility in a subject.

As used herein, the term “prognosis” with regards infertility refers to likely or expected development, progression and/or outcome of the infertility diagnosis.

In one example, the subject is at risk of infertility.

As used herein, a subject “at risk” of infertility may or may not have detectable infertility or symptoms of infertility. “At risk” denotes that a subject has one or more risk factors, which are measurable parameters that correlate with development of the disease or condition, as known in the art and/or described herein.

A subject is at risk if she has a higher risk of developing infertility than a control population. The control population may include one or more subjects selected at random from the general population (e.g., matched by age, gender, race and/or ethnicity) who have not suffered from or have a family history of infertility. A subject can be considered at risk if a “risk factor” associated with infertility is found to be associated with that subject. A risk factor can include any activity, trait, event or property associated with a given disorder, for example, through statistical or epidemiological studies on a population of subjects. A subject can thus be classified as being at risk even if studies identifying the underlying risk factors did not include the subject specifically.

In one example, the method of the present disclosure is performed before or after the onset of symptoms of infertility. Symptoms of infertility will be apparent to the skilled person and include, for example:
Age. Women in their late 30s and older are generally less fertile than women in their early 20s;
A history of endometriosis;
A history of adenomyosis;
Chronic diseases such as diabetes, lupus, arthritis, hypertension, and asthma;
Hormone imbalance;
Environmental factors including, cigarette smoking, drinking alcohol, and exposure to workplace hazards or toxins;
Too much body fat or very low body fat;
Abnormal Pap smears that have been treated with cryosurgery or cone biopsy;
Sexually transmitted diseases;
Fallopian tube disease;
Multiple miscarriages;
Fibroids;
Pelvic surgery; and
Abnormalities in the uterus that are present at birth or happen later in life.

As described above, methods of monitoring endometrial epithelial receptivity in a subject will be useful for the diagnosis and prognosis of infertility in a subject. In one example, the method of diagnosis and prognosis of infertility in the subject comprises determining the level of podocalyxin at multiple time points during the cycle. For example, the level of podocalyxin is determined at a time point during the ovarian cycle and/or at a time point during the uterine cycle. In one example, the level of podocalyxin is determined during the follicular phase, ovulation and/or the luteal phase. In a further example, the level of podocalyxin is determined during menstruation, the proliferative phase and/or the secretory phase. Furthermore, the level of podocalyxin may be determined at multiple time points in a single phase of a cycle. For example, the level of podocalyxin is determined at multiple points during the secretory phase of the uterine cycle.

### Medical Imaging

In addition to the methods described herein to monitor the level of podocalyxin, methods of monitoring podocalyxin in vivo can be used. For example, compounds that bind podocalyxin can be used in methods of imaging in vivo. In particular, compounds that bind podocalyxin and which are conjugated or bound to, and/or coated with, a detectable label, including contrasting agents, can be used in known medical imaging techniques.

For imaging podocalyxin in vivo, a detectable label may be any molecule or agent that can emit a signal that is detectable by imaging. For example, the detectable label may be a protein, a radioisotope, a fluorophore, a visible light emitting fluorophore, infrared light emitting fluorophore, a metal, a ferromagnetic substance, an electromagnetic emitting substance a substance with a specific MR spectroscopic signature, an X-ray absorbing or reflecting substance, or a sound altering substance.

Examples of imaging methods include MRI, MR spectroscopy, radiography, CT, ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

A variety of techniques for imaging are known to the person skilled in the art and/or are described herein. Any of these techniques can be applied in the context of the imaging methods of the present disclosure to measure a signal from the detectable label or contrasting agent conjugated to a compound that binds podocalyxin. For example, optical imaging is a widely used imaging modality. Examples include optical labeling of cellular components, and angiography such as fluorescein angiography and indocyanine green angiography. Examples of optical imaging agents include, for example, fluorescein, a fluorescein derivative, indocyanine green, Oregon green, a derivative of Oregon green derivative, rhodamine green, a derivative of rhodamine green, an eosin, an erytlirosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye.

In one example, the level of podocalyxin is detected using ultrasound. For example, the detectable label is an ultrasound agent. Suitable ultrasound agents will be apparent to the skilled person and/or are described herein. For example, the ultrasound agent is a microbubble-releasing agent (as described for example, in Willmann et al., 2017; Yeh et al., 2015; Abou-Elkacem et al., 2015; Tsuruta et al., 2014). In one example, a compound that detects podocalyxin is coupled to the microbubble. Various methods of coupling will be apparent to the skilled person and include, for example, covalent and non-covalent coupling. Following administration of the microbubble to the subject, the contact between the microbubble and its target (i.e., the endometrial epithelial cells) is enhanced by external application of an ultrasonic field. A microbubble, driven by an ultrasound field near its resonance frequency, experiences net primary and secondary ultrasound radiation forces, also known as Bjerknes forces. Ultrasound can displace microbubbles over significant distances (up to millimeters) in the direction of the ultrasound propagation and can cause attraction between microbubbles leading to aggregate formation. Thus, the microbubbles can be concentrated on the target.

The ability to monitor the level of podocalyxin in a subject in vivo and over the duration of the cycle and/or multiple cycles will assist in the diagnosis of infertility in the subject, allowing establishment of a therapeutic prognosis.

### Improving Endometrial Epithelial Receptivity and Treating Implantation Failure

The present inventors have also shown that persistent expression of podocalyxin in the endometrial luminal epithelium during the putative receptive phase is associated with implantation failure.

Currently in IVF practice, the endometrium is stimulated with progesterone prior to embryo transfer. However, there is no optimisation of drug type, dose and/or route prior to administration as there is no marker to assess the effectiveness of a hormonal preparation on endometrial epithelial receptivity.

The present inventors have shown that progesterone down-regulates podocalyxin in the luminal epithelium specifically for receptivity development.

Additionally, the present inventors have shown that microRNAs miR-145 and miR-199 are downstream regulators of progesterone in the suppression of podocalyxin during the establishment of endometrial epithelial receptivity.

Accordingly, the findings by the inventors provide the basis for using podocalyxin as a functional biomarker to optimize endometrial protocols for assisted reproductive technologies. For example, the findings by the inventors also provide the basis for methods of targeting podocalyxin to treat implantation failure.

In one example of the disclosure, methods as described herein according to any example of the disclosure involve reducing expression and/or the level of podocalyxin.

For example, the present disclosure provides methods of improving endometrial epithelial receptivity for embryo implantation in a subject comprising determining a level of podocalyxin in endometrial epithelial cells in the subject, and optionally based on the level of podocalyxin in the cells, administering to the subject a compound in an amount sufficient to reduce the level of podocalyxin in the endometrial epithelial cells.

For example, a subject may be in a pre-receptive state based on the level of podocalyxin in the cells and administration of a compound to the subject is sufficient to reduce the level of podocalyxin in the endometrial epithelial cells, thereby transitioning the subject to a receptive state.

The findings also provide the basis for methods of assessing effectiveness of a compound on improving endometrial epithelial receptivity for embryo implantation

As used herein, the term “compound” shall be understood to refer to any agent that is suitable for use in any method described herein. For example, a compound suitable for use in the present disclosure refers to any agent that alters the level (e.g., reduces the level) of podocalyxin in the endometrial epithelial cells. Compounds suitable for use in the present disclosure will be apparent to the skilled person and include, for example, any agent that down-regulates podocalyxin transcription or translation of the nucleic acid in endometrial luminal epithelial cells. For example, suitable compounds include, but are not limited to hormonal preparations and nucleic acids.

### Hormonal Preparations

In one example of any method described herein, the compound is a hormonal preparation. A variety of hormonal preparations suitable for use in the present disclosure will be apparent to the skilled person and include for example, progesterone, progestogen and an analog and combinations thereof.

### Nucleic Acids

In one example of any method described herein, the compound is a nucleic acid. For example, the nucleic acid is an antisense polynucleotide, a catalytic nucleic acid, an interfering RNA, a siRNA or a microRNA.

### Antisense Nucleic Acids

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann 1999).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding podocalyxin, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding podocalyxin. The full-length sequence complementary to the entire gene transcript may be used. The length can be 100-2000 nucleotides. The degree of identity of the antisense sequence to the targeted transcript should be at least 90%, for example, 95-100%.

### Catalytic Nucleic Acid

The term “catalytic nucleic acid” refers to a DNA molecule or DNA-containing molecule (also known in the art as a “deoxyribozyme” or “DNAzyme”) or a RNA or RNA-containing molecule (also known as a “ribozyme” or “RNAzyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are useful in this disclosure are a hammerhead ribozyme and a hairpin ribozyme.

### RNA Interference

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding podocalyxin. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is well within the capacity of a person skilled in the art, particularly considering WO99/32619, WO99/53050, WO99/49029 and WO01/34815.

The length of the sense and antisense sequences that hybridize should each be at least 19 contiguous nucleotides, such as at least 30 or 50 nucleotides, for example at least 100, 200, 500 or 1000 nucleotides. The full-length sequence corresponding to the entire gene transcript may be used. The lengths can be 100-2000 nucleotides. The degree of identity of the sense and antisense sequences to the targeted transcript should be at least 85%, for example, at least 90% such as, 95-100%.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search. Exemplary siRNA that reduce expression of podocalyxin are commercially available from Santa Cruz Biotechnology.

Short hairpin RNA (shRNA) that reduce expression of podocalyxin are also known in the art and commercially available from Santa Cruz Biotechnology.

MicroRNA (miRNA or miR) molecules comprise between 18 and 25 nucleotides in length, and is the product of cleavage of a pre-miRNA by the enzyme Dicer. “Pre-miRNA” or “pre-miR” means a non-coding RNA having a hairpin structure, which is the product of cleavage of a pri-miR by the double-stranded RNA-specific ribonuclease known as Drosha. Exemplary microRNAs that reduce podocalyxin expression will be apparent to the skilled person and/or described herein. For example, the nucleic acid is a microRNA, such as miR-199 or mir-145.

### Dosage and Administration

In one example, the method comprises determining the level of podocalyxin in endometrial epithelial cells in the subject and based on the level of podocalyxin in the cells, administering the compound in an amount sufficient to reduce the level of podocalyxin in the cells. For example, based on the level of podocalyxin in the subject one or more or all of dose, type of compound and/or route is modified.

The amount or dose of the compound required to reduce the level of podocalyxin in the cells will be apparent to the skilled person. The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the compound is administered at an initial (or loading) dose which is higher than subsequent (maintenance doses).

In some examples, a dose escalation regime is used, in which a compound is initially administered at a lower dose than used in subsequent doses.

A subject may be retreated with the compound based on the level of podocalyxin, by being given more than one exposure or set of doses, such as at least about two exposures, for example, from about 2 to 60 exposures, and more particularly about 2 to 40 exposures, most particularly, about 2 to 20 exposures.

Administration of a compound according to the methods of the present disclosure can be continuous or intermittent, depending, for example, on the recipient's physiological condition, whether the purpose of the administration is therapeutic or prophylactic, and other factors known to skilled practitioners. The administration may be essentially continuous over a preselected period of time or may be in a series of spaced doses, e.g., either during or after development of a condition.

As described above, methods of monitoring endometrial epithelial receptivity in a subject will be useful for monitoring and determining the effectiveness of a compound in improving the endometrial epithelial receptivity. Monitoring endometrial epithelial receptivity in a subject during administration of the compound will also assist in optimising the treatment regimen for the subject. For example, the level of podocalyxin is determined before and/or after administration of the compound and the dose, route and/or type of compound administered adjusted accordingly.

It will be apparent to the skilled person that optimisation of the dose, route and/or type of compound will assist in improving endometrial epithelial receptivity in the subject and maximise the probability of implantation.

### EXAMPLES

### Example 1: Materials and Methods

### Human Endometrial Tissues for Isolation of Primary Endometrial Epithelial Cells

Ethics approval was obtained from the Human Ethics Committee at Monash Medical Centre (Melbourne, Australia), and all patients provided informed written consent. Endometrial biopsies were obtained from women undergoing hysteroscopy dilatation, curettage or assessment of tubal patency. The menstrual cycle stage was confirmed by routine histologic dating of the tissue.

### Isolation of Primary Human Endometrial Epithelial Cells (HEECs)

Tissues from the proliferative phase (days 6-14) were collected into Dulbecco's modified Eagle's medium/F12 (DMEM/F12, Thermo Fisher Scientific, Mass., USA), and cells were isolated within 24 h of collection. Cells were isolated by enzymatic digestion and filtration as previously described (Marwood et al., 2009). Briefly, endometrial tissue samples were digested with collagenase from Clostridium histolyticum (7.5 U/ml; Sigma) and DNase 1 (2000 U/ml; Roche, Castle Hill, NSW, Australia) in a 37° C. water bath with constant shaking for 2×20 mins. The digestion reaction was quenched with complete medium containing DMEM/F12 supplemented with 10% fetal bovine serum (FBS) (Bovogen Biologicals Pty Ltd, AUS) and 1% antibiotic-antimycotic (Sigma), and filtered through a 45 μm nylon mesh. The human endometrial epithelial cells (HEECs) retained on the mesh were rinsed with 10 ml of PBS into a new tube and centrifuged at 1000 rpm for 5 min at RT; the cell pellet was resuspended in DMEM/F12 supplemented with 10% FBS and 1% antibiotic-antimycotic, seeded into a 24-well plate and incubated at 37° C. under 5% CO₂ in a humidified incubator.

The following day, any unattached cells and red blood cells were removed and the attached HEECs were replenished with fresh medium every 3 days until 90-95% confluency was reached. The HEECs were then used to investigate the hormonal regulation of PCX.

Isolation of the Plasma Membrane Proteins from Primary HEECs

Primary HEECs, isolated as above but without further culture, were lysed with ice cold lysis buffer [25 mM imidazole and 100 mM NaCl pH 7.0 containing protease inhibitors cocktail (Roche)] and passed through a 27.5-gauge needle and syringe seven times, and centrifuged at 15,000 g for 5 min at 4° C. The supernatant was incubated with 100 mM Na₂CO₃ on ice for 1 h (with vortex every 15 mins) and centrifuged at 100,000 g for 60 min at 4° C. to collect the pellet containing the plasma membrane.

The plasma membrane proteins (100 μg) were processed using filter-aided sample preparation (FASP) columns (Expedeon Inc., CA). The tryptic peptides from FASP columns were collected by centrifugation and desalted on C₁₈ StageTips for mass spectrometry analysis.

### Mass Spectrometry Analysis

The extracted peptides were injected and separated by nano-flow reversed-phase liquid chromatography on a nano ultra-performance liquid chromatography (UPLC) system (Waters nanoAcquity, Waters, Milford, Mass.) using a nanoAcquity C18 150×0.075 mm I.D. column (Waters) with a linear 60 min gradient set at a flow rate of 0.4 μL/min from 95% solvent A (0.1% Formic acid in milliQ water) to 100% solvent B (0.1% Formic acid, 80% acetonitrile (Mallinckrodt Baker, Center Valley, Pa.), and 20% milliQ water). The nano UPLC was coupled online to a Q-Exactive mass spectrometer equipped with a nano-electrospray ion source (Thermo Fisher Scientific, Bremen, Germany) set to acquire full scan (70000 resolution) and top-10 multiply charged species selected for fragmentation using the high-energy collision disassociation with single-charged species were ignored. Fragment ions were analyzed with the resolution set at 17500, with the ion threshold set to 1e5 intensity. The activation time was set to 30 ms, and the normalized collision energy was stepped ±20% and set to 26. Raw files consisting of full-scan MS and high resolution MS/MS spectra were searched using the Maxquant algorithm (version 1.4). Trypsin was set to two missed cleavages, and files were searched with variable modifications set for oxidized methionine, and fixed modification in the form of carbamidomethyl Cys residues (using the default Maxquant settings with the cut-off score and delta score for modified peptides set at 40 and 17, respectively). All MS/MS samples were also analyzed using Mascot (Matrix Science, London, UK; version 2.4.1). Mascot was searched with a fragment ion mass tolerance of 0.040 Da and a parent ion tolerance of 20 PPM. Carbamidomethyl of cysteine was specified in Mascot as a fixed modification. Oxidation of methionine and acetyl of the N-terminus were specified in Mascot as variable modifications.

Reported peptides were then analysed in Scaffold (version Scaffold4.4.1.1, Proteome Software Inc., Portland, Oreg.). Peptide identifications were accepted if they could be established at greater than 95% probability by the Scaffold Local FDR algorithm. Protein identifications were accepted if they could be established at greater than 90% probability and contained at least one identified peptide from each sample.

### Culture of Primary HEECs and Hormonal Treatment

Confluent HEECs were seeded into 12 well-plates or glass coverslips for 5 hr at 37° C. under 5% CO₂ in a humidified incubator, then primed overnight with 10 nM of 17β-estradiol (E) (Sigma) in complete medium containing DMEM/F12 supplemented with 10% charcoal stripped FBS. The following day, the E priming medium was removed and the cells were replenished with fresh complete medium containing 10 nM E without or with 1 μM medroxyprogestrone-17-acetate (P) (Sigma), which were designated as E and E+P respectively. Cells were treated with E or E+P for a time course of 48 h, 72 h and 96 h. At the conclusion of each time point, cells were either washed twice with PBS, trypsinised, pelleted and snapped frozen for RNA isolation, or scraped with ice cold PBS for protein isolation, or fixed with ice cold 100% methanol or 4% (W/v) paraformaldehyde (PFA) for immunofluorescence.

Endometrial Tissues from Normal Healthy Women for Localization of PCX Protein

Endometrial tissues were obtained in accordance with the Ethics Committee for the Protection of Human Subjects at the University of North Carolina and Greenville Hospital System. Biopsies were taken from normal healthy women at different stages of the menstrual cycle with 25-35 day intermenstrual intervals (n=22). Exclusion criteria include: age <18 or >35 years, body mass index >29, abnormal PAP test within the past year, attempting or currently pregnant, sexually active and not using condoms, with an intrauterine device in place, history of pregnancy loss, uterine abnormalities such as fibroids, breastfeeding, medication that influences endometrial morphology, known cervical stenosis, allergy to betadine and underlying medical disorders. Cycle day was determined by the first day of menstruation. Urinary LH was determined by a home test kit (Ovuquick One Step, Conception Technologies, San Diego, Calif.). Endometrial samples were classified by the reported cycle day and by the number of days after the LH surge (LH+). Day of cycle was also confirmed by hematoxylin and eosin. Endometrial biopsies were obtained from proliferative (n=5), early-secretory (n=6, LH+4-5), mid-secretory (n=6, LH+7-10) and late-secretory (n=5, LH+12-13) phases of the menstrual cycle. All endometrial biopsies were fixed in formalin and embedded in paraffin.

### Immunohistochemical Localization of PCX in Human Endometrial Tissues

Endometrial sections (5 μm) were deparaffinised in histosol, rehydrated and antigen was retrieved by microwaving (10 min at high power in 0.01M citrate buffer pH 6.0). Endogenous peroxidase was quenched with 3% H₂O₂ in methanol for 10 min and non-specific binding was blocked with 15% horse serum in high salt TBS (0.3M NaCl, 0.05M Tris base pH 7.6) containing 0.1% Tween 20 for 20 min. The sections were incubated for 1 h at 37° C. with primary PCX antibody (Ab2, details on P42, 2 μg/ml) in 10% fetal calf serum in high salt TBS containing 0.1% Tween 20. Mouse IgG (Dako) replaced the primary antibody in the negative control. Sections were washed and appropriate biotinylated secondary antibodies (Vector laboratories, Inc. USA) were applied for 30 min at room temperature. Signals were amplified with StreptABC/HRP (Dako) for 30 min at room temperature and visualized with diaminobenzidine (Dako). Cell nuclei were stained with haematoxylin (blue) and sections were mounted with DPX reagent.

### Quantification of PCX Staining in Endometrial Tissues

Slides were blindly analysed using image analysis software Fiji 1.51o (National Institutes of Health, Bethesda, Md.). For every section, three representative images of LE, GE and BV were taken. Each image was analysed by background subtraction using the rolling ball algorithm and “colour deconvolution” using the built in vector hematoxylin and diaminobenzidine (HDAB) plugin, which separated the image into 3 panels: hematoxylin, DAB and background. On the DAB panel (showing PCX staining), the region of interest was selected with the freehand tool and its gray value measured. The mean gray value per section was calculated from three representative images and converted to optical density unit [ODU=log₁₀(255/mean gray value), which was used to express the PCX staining intensity.

### Western Blot Analysis

Cells were lysed with 50 mM Tris-HCl pH7.4, 150 mM NaCl, 1 mM EGTA, 2 mM EDTA, 1% Triton X containing protease inhibitor cocktail (Roche). Lysates were frozen on dry ice for 10 mins, then thawed at room temperature for a further 5 mins. This freeze-thaw cycle was repeated three times. Samples were then centrifuged at 14000 rpm for mins at 4° C. and the supernatant containing proteins were separated on a 10% SDS-polyacrylamide gel and transferred onto polyvinyl difluoride membrane (GE Healthcare, Rydalmere, NSW, Australia). The membrane was blocked with 5% BSA in Tris-buffered saline [10 mmol/L Tris (pH7.5) and 0.14 mol/L NaCl] with 0.02% Tween20. Three PCX antibodies were used for western blot analysis: Ab1 was raised against the highly glycosylated mucin region aa 23-427 (AF1658, R&D Systems Minneapolis, Minn.); Ab2 was raised against a portion of the extracellular domain aa 251-427 (3D3, Santa Cruz, Dallas, Tex.) (Kershaw et al., 1997); Ab3 was raised against the extracellular, transmembrane and intracellular part of PCX aa 300-500 (EPR9518, Abcam, Cambridge, UK) (Kershaw et al., 1997). Appropriate secondary antibodies included goat IgG-HRP, mouse IgG-HRP or rabbit IgG-HRP (Dako, Victoria, Australia). Bands were visualized using the Lumi-light enhancer solution (Roche). Membranes were probed for R-actin (Cell Signaling Technology, Danvers, Mass.) for loading control. Recombinant human PCX which contained the extracellular part of PCX (rPCX, aa23-427, R&D Systems) and human umbilical vein endothelial cells (HUVECs) served as positive controls. This experiment was repeated four times.

### Transient Knockdown of PCX in Ishikawa Cells

Ishikawa cells (a generous gift by Professor Masato Nishida of National Hospital Organization, Kasumigaura Medical Center, Ibaraki-ken, Japan) were cultured overnight at 5.6×10⁵ cells/well in a 6-well plate in complete medium containing modified Eagle's medium (MEM, Life Technologies, Carlsbad, Calif.) supplemented with 10% (v/v) FBS, 1% antibiotic-antimycotic and 1% L-glutamine. The following day, cells were replenished with Opti-MEM medium for transfection. PCX-unique 27mer siRNA duplex (SR303611B) and the universal scrambled negative control siRNA duplex (SR30004) were obtained from Origene (Rockville, Md.). One microliter of master mix containing control or PCX siRNA (20 μM stock) was added into 250 μl Opti-MEM medium, 4 μl of lipofectamine transfection reagent was diluted in 250 μl Opti-MEM medium, they were then mixed together and added to the wells. After 24 h incubation at 37° C., cells were changed to complete media and cultured for another 24 h and PCX knockdown (KD) confirmed by qRT-PCR and western blot.

### Stable Overexpression of PCX in Ishikawa Cells

An expression construct of human PCX open reading (RC210816) and the empty pCMV6 (control plasmid) were purchased from Origene. Ishikawa cells were grown on a 6-well plate to confluence in MEM medium supplemented with 10% FBS, 1% antibiotic-antimycotic and 1% L-glutamine, then washed with PBS and replenished with Opti-MEM medium the following day for transfection as previously described (Heng et al., 2015). A mastermix of plasmid DNA (containing PCX or control) and lipofectamine transfection reagent (Life Technologies) in a 1:3 ratio in Opti-MEM medium (Life Technologies) was added to the well (1 μg DNA/well) and incubated for 24 h at 37° C. under 5% CO₂ in a humidified incubator. The cells were replenished with fresh Opti-MEM medium and cultured for another 24 h, then transferred into a 10 cm Petri dish containing complete medium with 2% geneticin. After reaching ˜90% confluency, cells were trypsinised, seeded very sparsely in 25 cm petri-dishes (˜20,000 cells/dish), and cultured until individual colonies formed. Each colony was then trypsinised and transferred into 96-well plates. Colonies that grew well were up-scaled sequentially to larger wells of 48-, 24-, 12- and 6-well plates. The final colonies were confirmed by qRT-PCR and western blot analysis.

Confirmation of PCX in Ishikawa Cells by qRT-PCR

Total RNA was extracted from primary HEECs, HUVECs and Ishikawa cells (PCX-OE, PCX-KD and controls) using the RNeasy Mini Kit (Qiagen, Hilden, Germany), and treated with TURBO DNA-free kit (Invitrogen, Vilnius, Lithuania). Total RNA (500 ng) was reverse transcribed using the Superscript III First-Strand Synthesis System (Invitrogen, Carlsbad, Calif.) per manufacturer's instructions. qRT-PCR was performed as above for PCX. Quantitative PCR was performed on the Applied Biosystems 7900HT fast real-time PCR system, using Power SYBR Green PCR master mix (Applied Biosystems, Warrington, UK) and primers listed in Table 1.

TABLE 1
Primer sequences

| | Primer sequence (5′-3′) | |
|---|---|---|
| Gene | Forward | Reverse |
| PODXL | GAGCAGTCAAAGCCAC | TGGTCCCCTAGCTTCATGTC |
| (PCX) | CTTC | |
| CDH1 | GAAGGTGACAGAGCCT | GATCGGTTACCGTGATCAAA |
| | CTGGAT | ATC |
| TJP1 | GGGAACAACATACAGT | CCCCACTCTGAAAATGAGGA |
| | GACGC | |
| CLDN4 | CCC CGA GAG | AGC GTC CAC GGG AGT |
| | AGAGTG CCC TG | TGA GGA |
| OCLN | CTCTCTCAGCCAGCCT | GTTCCATAGCCTCTGTCCCA |
| | ACTC | |
| WNT7A | TGCCCGGACTCTCATG | GTGTGGTCCAGCACGTCTTG |
| | AAC | |
| LEFTY2 | CTGGACCTCAGGGACT | TCAATGTACATCTCCTGGCG |
| | ATGG | |
| LIF | TGCCAATGCCCTCTTT | GTTGACAGCCCAGCTTCTTC |
| | ATTC | |
| CSF1 | TAGCCACATGATTGGG | CTCAAATGTAATTTGGCACG |
| | AGTGGA | AGGTC |
| ERBB4 | GATGATCGTATGAAGC | CGGTATACAAACTGGTTCCT |
| | TTCCCA | ATTC |
| FGF2 | CGGATGGGGGTAGTGA | ATCTTGAGGTGGAAGGGTCT |
| | GCA | |
| TGFB1 | CAACAATTCCTGGCGA | GCTAAGGCGAAAGCCCTCAA |
| | TACCT | T |
| MMP14 | GCAGAAGTTTTACGGC | TCGAACATTGGCCTTGATCT |
| | TTGCA | C |
| YWHAZ | CCGCCAGGACAAACCA | ACTTTTGGTACATTGTGGCT |
| | GTAT | TCAA |
| 18S | CGGCTACCACATCCAA | GCTGGAATTACCGCGGCT |
| | GGAA | |

### Immunofluorescence Analysis of PCX in Primary HEECs

Cells grown on glass coverslips were fixed with ice cold methanol for 10 min and rinsed 3 times with PBS. Cells were permeabilised with 0.1% Triton-X100 in PBS for min and blocked with 15% horse serum and 2% human serum in PBS for 30 min. Cells were incubated with Ab1 (at 6 μg/ml) overnight at 4° C. in 5% horse serum/PBS. The following day, cells were washed for 3 times 5 min with PBS containing 0.2% Tween20 and incubated with horse anti-goat biotinylated secondary antibody (at 10 μg/ml, Vector Laboratories, Peterborough, UK) for 1 h at RT, then with streptavidin conjugated Alexa Fluor 488 (at 10 μg/ml, Invitrogen, Carlsbad, Calif.) for 2 h at RT. The nuclei were stained with DAPI (at 0.5 μg/ml, Sigma). The signal was visualized by fluorescence microscopy (Olympus Optical, Tokyo, Japan).

### Analysis of Ishikawa Cell Adhesion to Fibronectin

Analysis of Ishikawa cell adhesion to fibronectin was performed as previously described in Heng et al., 2015.

Briefly, 96-well plates were coated with 10 μg/ml fibronectin (Corning Life Sciences, Tewksbury, Mass.) and Ishikawa cells (PCX-OE, PCX-KD or controls) were added to the fibronectin-coated wells (2×10⁴ cells/well) and incubated for 90 min at 37° C. Non-adherent cells were removed and the wells were gently washed with PBS+ (containing Ca2+Mg2+), and incubated with 0.2% crystal violet in 10% ethanol for 5 min at RT without agitation. After removing the crystal violet solution, each well was washed 3 times with PBS+ to remove all remaining crystal violet stain. The bound cells (stained purple) were solubilized with solubilization buffer (a 50/50 mix of 0.1 M NaH₂PO₄, pH 4.5 and 50% ethanol) for 5 min on a rocker at 250 rpm at RT. The absorbance at 560 nm was measured with an Envision plate reader (PerkinElmer, Waltham, Mass.). Wells with media alone were included as negative control.

Collection and Isolation of Trophoblast Villi from Term Placenta

Ethics approval was obtained from Monash Health Human Research Ethics Committee and all subjects provided informed written consent for the collection of placental samples from elective caesarean birth of healthy term singleton pregnancies.

Trophoblasts were isolated as previously described (Wallace et al., 2017). In brief, placental cotyledons were excised and washed with Hank's balanced salt solution, the villi (˜25 g) were scraped from the cotyledons and digested with buffer containing DMEM low glucose, 1% penicillin, 1% streptomycin, 0.25% trypsin, 0.25% grade II dispase, 0.1 mg/ml DNase 1 in a 37° C. shaking water bath for 15 mins. After 3 cycles of digestion, the cell suspension was separated by Percoll gradient centrifugation, trophoblast cells were collected and cultured in DMEM with 10% FBS, 1% antibiotic-antimycotic at 37° C. under 8% O₂ overnight.

### Preparation of Primary Trophoblast Spheroids

AggreWell™ 400 plate (Stemcell Technologies, Vancouver, Canada) was pre-rinsed with 2 ml anti-adherence rinsing solution, centrifuged at 2000 g for 5 min at RT, and washed with 2 ml of DMEM/F12 medium as per manufacturer's protocol. Primary trophoblast cells were trypsinised, and resuspended in EB formation medium (Stemcell) and 9.6×10⁵ cells/ml were transfer into each well of the AggreWell™ 400 plate. Each well was topped up with EB medium to a total of 2 ml/well, centrifuged at 100 g for 5 min at RT and incubated at 37° C. under 5% CO₂ in a humidified incubator for 48 h.

For spheroid invasion studies, 5 μl per 1 ml of either vibrant cell-labelling solution DiO or DiI (Thermo Fisher Scientific) was added to the medium prior to centrifugation. Trophoblast spheroids of approximately 100 μm in diameter formed after this 48 h incubation. The spheroids were dislodged from the Aggrewell plate by manual pipetting, passed through a 40 μm cell strainer to remove spheroids less than ˜100 μM in size. The final spheroids were collected into a low binding 6-well plate by inverting the cell strainer on top of the plate and rinsing it with DMEM/F12 supplemented with 10% FBS, 1% antibiotic-antimycotic for attachment and invasion experiments.

### Assessment of Primary Trophoblast Spheroid Attachment to Ishikawa Monolayer

Control or PCX-OE Ishikawa cells were cultured overnight at 37° C. in a 96-well flat-bottom plate to form a monolayer. Concurrently prepared primary trophoblast spheroids were then transferred onto the top of Ishikawa monolayer (approximately 30 spheroids per well in 100 μl of medium), and incubated for 1 h, 2 h, 4 h, 6 h, 12 h or 24 h respectively. The exact number of trophoblast spheroids added in each well was counted before the wells were washed 3 times with PBS to remove unattached spheroids. Fresh culture medium was added and the attached spheroids in each well were counted and the attachment rate (percentage of attached/pre-washed spheroids) was calculated. Each experiment was based on the average of triplicate wells and the final data was expressed as mean±SD of 3-5 independent experiments.

### Assessment of Primary Trophoblast Spheroid Traversing Through Ishikawa Monolayer

Glass coverslip slides containing 8-well chambers (Sarstedt, Germany) were coated with a mixture of collagen type 1 (Merck-Millipore, USA) and human fibronectin (Corning, USA) in DMEM for 10 min at RT then 1 h at 37° C. Control and PCX-OE Ishikawa cells were cultured on top of the matrix in conditioned medium containing G418 to form a monolayer overnight at 37° C., 5% CO₂. The following day the conditioned medium was removed from each well and replenished with conditioned medium containing either vybrant cell-labeling solution DiO or DiI depending on the combination used to stain the spheroids (Thermo Fisher Scientific, 5 μl per 1 ml of medium) and incubated for another 24 hr. Medium containing the vybrant solution was removed and the wells were washed twice with PBS, approximately 1-3 spheroids in 100 μl of trophoblast conditioned medium (DMEM/F12 supplemented with 10% FBS and 1% antibiotic-antimycotic) were then transferred into each chamber of either control or PCX-OE Ishikawa monolayers, and co-cultured for 24 h or 48 h at 37° C., 5% CO₂. The chambers were then imaged using confocal microscopy fitted with a 37° C., 5% CO₂ incubator (Olympus, Japan).

### Assessment of Human Embryo Attachment

Control or PCX-OE Ishikawa cells were cultured in conditioned medium containing G418 overnight at 37° C., 5% CO₂ in 96-well flat bottom plates to form a monolayer. Prior to co-culture with human embryos, the conditioned medium was removed and replenished with fresh medium without G418 and left to equilibrate for 4 h at 37° C., 5% CO₂.

The use of cryopreserved human embryos collected at the Centre for Reproductive Medicine (CRG, UZ Brussels, Belgium) were approved by the Institute Ethical Committee and the Federal Committee for Scientific Research on Human Embryos in vitro. With written informed consent from patients, embryos used for this particular study were from embryos donated to research after the legally determined cryopreservation period of five years. Good quality vitrified 5 day post fertilization (dpf) blastocysts, which are full and expanding blastocysts with A or B scoring for both inner cell mass (ICM) and trophectoderm (TE) according to Gardner and Schoolcraft criteria (Gardner et al., 1999) were warmed using the Vitrification Thaw Kit (Vit Kit-Thaw, Irvine Scientific, USA) following manufacturer's protocol and transferred into 25 μl droplets of Origio blastocyst medium (Origio, The Netherlands) for recovery at 37° C. with 20% O₂, 6% CO₂ and 89% N₂. A large hole was made in the zona pellucida (ZP) of each blastocyst, approximately a quarter in length using a laser to assist with embryo hatching overnight. Based on morphological scoring, only good quality 6dp embryos hatched from the ZP were used for further experiments. Each embryo was removed from their culture droplet, rinsed with Ishikawa conditioned medium (without G418), transferred to the top of control and PCX-OE monolayer and co-cultured for 15 h and 24 h at 37° C., 5% CO₂. The rate of embryo attachment to Ishikawa monolayer was assessed under a stereological light microscope (Nikon, Japan) where the medium was gently pipetted up and down 3-4 times using a 200 μl tip at the different time points. Free floating embryos were considered as unattached. The attachment rate was calculated as the percentage of the number of attached embryo over the total number of transferred embryos. The final data was the average value of 3 independent experiments.

### Assessment of Human Embryo Traversing Through Ishikawa Monolayer

A monolayer of control and PCX-OE Ishikawa cells was prepared on a layer of matrix on glass coverslip slides containing 8-well chambers as previously described for the assessment of trophoblast spheroid traversing the Ishikawa monolayer. This model also used 6dpf embryos with the same selection criteria as the above attachment assay, but instead of warming 5dpf embryos, 3dpf embryos were warmed as prior to setting up the invasion model embryos need to be stained with either DiO or DiI. Thus, good quality vitrified 3dpf blastocysts, at compaction C1 and C2 stages according to Gardner and Schoolcraft criteria (Gardner et al., 1999), were warmed using the Vitrification Thaw Kit (Vit Kit-Thaw, Irvine Scientific, USA) following manufacturer's protocol and transferred into 25 μl droplets of Origio blastocyst medium (Origio, The Netherlands) for recovery at 37° C. with 20% O₂, 6% CO₂ and 89% N₂. A large hole was made in the zona pellucida (ZP) of each 4dpf blastocyst using a laser and left to recover overnight. The next day good quality 5dpf blastocysts were transferred into culture droplets containing vybrant cell-labeling solution DiO or DiI (Thermo Fisher Scientific, 10 μl per 1 ml of medium) and incubated for 24 h at 37° C. with 20% O₂, 6% CO₂ and 89% N₂. Based on morphological scoring, only good quality 6dpf embryos hatched from the ZP were used for the invasion assay experiments. Each embryo was removed from the culture droplet, rinsed with Ishikawa conditioned medium (without G418), transferred to the top of control or PCX-OE monolayer and co-cultured for 24 h at 37° C., 5% CO₂. Following the co-culture, each chamber was imaged using confocal microscopy (Zesis, Germany).

### Confocal Imaging Analysis of Trophoblast Spheroid and Human Embryo Invasion

Surface mapping for primary trophoblast spheroids or human embryos co-cultured with Ishikawa monolayers (control or PCX-OE) was performed using the Imaris software (version 9.2.1, Bitplane, AG). The extent of invasion was determined by the volume of spheroid/embryo that invaded through the monolayer and was present beneath the Ishikawa monolayer.

### RNAseq of Control and PCX-OE Ishikawa Cells

Ishikawa cells were cultured overnight at 5.6×10⁵ cells/well in a 6-well plate in MEM medium supplemented with 10% FBS, 1% antibiotic-antimycotic and 1% L-glutamine. The following day, cells were washed with PBS and total RNA was isolated from control and PCX-OE Ishikawa cells using the RNeasy Mini Kit (Qiagen), and treated with TURBO DNA-free kit (Invitrogen).

Initial raw read processing was performed and raw 75 bp single-end FASTQ reads were assessed for quality using FastQC (Andrews 2010) and results aggregated using R/Bioconductor package ngsReports (Ward et al. 2018). Reads were then trimmed for sequence adapters using AdapterRemoval (Schubert et al. 2016) and aligned to the human genome GRCh37 using the RNA-seq alignment algorithm STAR (Dobin et al. 2013). After alignment, mapped sequence reads were summarised to the GRCh37.p13 (NCBI:GCA_000001405.14 2013-09) gene intervals using featureCounts (Liao et al. 2014), and count table transferred to the R statistical programming environment for expression analysis. Effect of sequence duplicates were also investigated using the function MarkDuplicates from the Picard tools package (http://broadinstitute.github.io/picard).

Gene expression analyses were carried out in R using Bioconductor packages edgeR (Robinson et al. 2009; McCarthy et al. 2012) and limma (Richie et al. 2015). Gene counts were filtered for low expression counts by removing genes with less than 1 count per million (cpm) in more than two samples and then normalised by the method of trimmed mean of M-values (TMM; Robinson & Oshlack, 2010). Differential gene expression was carried out on log-CPM counts and precision weights available from the voom function in limma (Law et al. 2014), with linear modelling and empirical Bayes moderation.

Annotation of results were carried out using Ensembl annotations (http://grch37.ensembl.org) available in biomaRt (Durinck et al. 2009), and expression results displayed in heatmaps using the pheatmap package (Kolde 2019). Additional pathway and gene set enrichment analyses were carried out using clusterProfiler (Yu et al. 2012) and msigdbr (Dolgalev 2018) on KEGG pathway (https://www.genome.jp/kegg/pathway.html) and Molecular Signature (MSigDB) databases (Liberzon et al. 2015).

### Immunofluorescence of Junctional Proteins in Ishikawa Cells

Control and PCX-OE Ishikawa cells were grown on glass coverslips, fixed in either 4% (w/v) paraformaldehyde (for analysis of E-cadherin, Wnt-7A, claudin-4 and ZO-1), or in 100% methanol (for occludin). Cells were then blocked at RT with protocols optimized for individual antibodies (E-cadherin: 10% horse serum and 1% BSA in PBS for 1 h; Wnt-7A: 10% horse serum in PBS for 2 h; Claudin-4: 10% horse serum, 2% human serum, 0.1% fish skin gelatin and 0.1% Triton X-100 in PBS containing 0.2% Tween20 for 1 h; ZO-1: 1% BSA in PBS for 2 h; and occludin: 10% goat serum, 2% human serum, 0.1% fish skin gelatin and 0.1% Triton X-100 in PBS containing 0.2% Tween20 for 1 h.

Cells were probed overnight at 4° C. with the primary antibodies, E-cadherin (2 μg/ml, ab1416, Abcam), Wnt-7A (6 μg/ml, AF3008, R&D), claudin-4 (6 μg/ml, sc-376643, Santa Cruz), occludin (1 μg/ml, 71-1500, Thermo Fisher) and ZO-1 (10 μg/ml, 61-7300, Thermo Fisher). The following day, the cells were washed 3 times 15 min in PBS, incubated with the appropriate biotinylated secondary antibodies for 1 h at RT, followed by the addition of streptavidin conjugated Alexa Fluor 488 for 1 h at RT. The nuclei were stained with DAPI for 5 min at RT (0.5 μg/ml in PBS, Sigma). The fluorescence signal was visualized by fluorescence microscopy (Olympus Optical, Tokyo, Japan).

### Assessment of Ishikawa Monolayer Permeability

For measurement of both trans-epithelial electrical resistance (TER) and the transport of fluorescein isothiocyanate (FITC)-conjugated dextran 40,000 from the upper to the bottom wells, permeable transwell inserts (6.5 mm, 0.4 μm pore, Corning, N.Y.) coated with 10 μg/ml fibronectin (BD Biosciences, NSW, AUST) were used. Control and PCX-OE Ishikawa cells were seeded (6×10⁴ cells per insert) and incubated overnight with complete media containing 2% G418. TER was measured after 96 h using a Millipore MilliCell-Electrical Resistance System (Millipore, Mass.). The upper chamber was replaced with serum-free media and lower chamber contained complete media (both containing 2% G418). The cells were maintained at 37° C. using a warming plate throughout TER measurements. Four TER readings (ohm×cm²) were taken from each well and readings from duplicate wells averaged to obtain the raw TER. The final value was obtained by subtracting the background TER from wells that contained no cells in the same experiment.

To measure the passage of FITC dextran, control and PCX-OE Ishikawa cells were also cultured for 96 h. Afterwards, fresh complete medium containing 2% G418 was added to bottom chamber and fresh complete medium containing 2% G418 and FITC dextran (1 mg/ml, Sigma) was added to the upper chamber. The cells were incubated at 37° C. for 2 h, the media from the bottom chamber was collected and diluted 1:5 in PBS for fluorescence measurements at 485/535 nm (Clariostar, BMG LabTech, Victoria, Australia). The final fluorescence reading was obtained after subtracting the background (PBS only) and the data were expressed as mean±SD of four independent experiments.

Endometrial Tissues Obtained from the Endometrial Scratch Procedure

A cohort of archived endometrial tissues biopsied during the endometrial scratch procedure during fertility treatment were retrieved for immunohistochemical analysis of PCX in the luminal epithelium. All biopsies were taken in the mid-secretory phase (d20-24) in the natural cycle of the month immediately prior to IVF treatment. All patients experienced ≥2 cycles of implantation failure prior to undergoing the scratch procedure, and a single high quality embryo (grade A-C) was transferred in the immediate next cycle after the scratch. Samples were biopsied between 2012-2016 at Monash IVF (Clayton, VIC, Australia) and analysed/archived by Anatpath Services (Gardenvale, VIC, Australia) after fixing in formalin. Ethics approval for retrieving such tissues from Anatpath for this study was obtained from Monash Health.

### Statistics

GraphPad Prism version 7.00 (GraphPad Software, San Diego, Calif.) was used for statistical analysis of unpaired t-test, one-way ANOVA or Fisher's exact test where appropriate, and data were expressed as mean±SD. Significance was defined as *P<0.05, **P≤0.005, ***P≤0.0005 and ****P≤0.0001.

### Example 2: Proteomic Identification of Podocalyxin in Primary Human Endometrial Epithelial Cells

Primary endometrial epithelial cells (HEECs) from human endometrial tissues were isolated and enriched for plasma membrane proteins as described in Example 1.

The resulting proteins were analysed by mass spectrometry and a total of 250 proteins were identified (Table 2). Of these, 47 were deemed to be cell membrane proteins, 10 of which were associated with cell adhesion including podocalyxin (PCX).

To confirm the proteomic finding, total cell lysates of primary HEECs isolated from the proliferative phase endometrium (as for the proteomic study) were analysed by western blot using 3 antibodies against different regions of human PCX.

A dominant band of ˜150 kDa was detected by all 3 antibodies with compatible levels in both cell types. Ab1 detected an additional fainter band of ˜80 kDa in both HUVECs and HEECs, whereas Ab2 recognized additional bands of ˜45, 37 and 30 kDa primarily in HUVECs. The size of rPCX was slightly <150 kDa, consistent with it containing the extracellular domain only. These data confirmed that PCX was expressed in the proliferative phase endometrial epithelial cells.

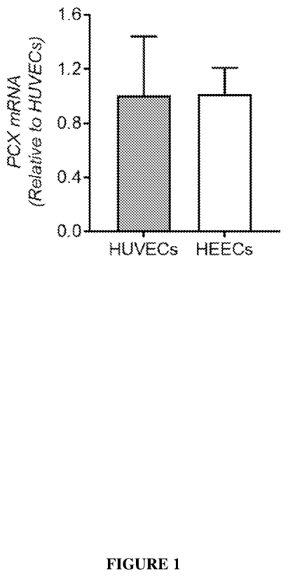

RT-PCR analysis further validated this finding, detecting compatible levels of PCX mRNA transcripts in HEECs and HUVECs (positive control; FIG. 1).

TABLE 2
Proteins identified by LC-MS/MS analysis of plasma membrane-enriched proteins of human endometrial
epithelial cells (HEECs) isolated from the proliferative phase of the menstrual cycle

| | | | | | % Probability | | | |
|---|---|---|---|---|---|---|---|---|
| | | Protein | | | (Total unique peptides) | | | |
| Cellular | Category/ | accession | | | Samples | | | Protein |
| localisation | Function | number | Gene name | Protein name | 1 | 2 | 3 | # |
| Cell | Cell adhesion | O00592 | PODXL | Podocalyxin | 99(1) | 99(1) | 100(3) | 1 |
| membrane | | P23229 | ITGA6 | Integrin alpha-6 | 100(9) | 100(8) | 100(9) | 2 |
| | | P06756 | ITGAV | Integrin alpha V protein | 100(7) | 100(2) | 100(8) | 3 |
| | | P05106 | ITGB3 | Integrin beta-3 | 100(5) | 100(2) | 100(7) | 4 |
| | | P21926 | CD9 | CD9 antigen | 100(2) | 100(3) | 100(1) | 5 |
| | | P56199 | ITGA1 | Integrin alpha-1 | 100(5) | 100(6) | 100(6) | 6 |
| | | Q9Y639 | NPTN | Neuroplastin | 100(3) | 99(1) | 99(1) | 7 |
| | | P15941 | MUC1 | Mucin-1 | 98(1) | 99(1) | | 8 |
| | | P05556 | ITGB1 | Integrin beta-1 | 100(9) | 100(11) | 100(12) | 9 |
| | | P16422 | EPCAM | Epithelial cell adhesion | 100(3) | 100(3) | 100(2) | 10 |
| | | | | molecule | | | | |
| | Actin | P04083 | ANXA1 | Annexin | 100(7) | 100(9) | 100(3) | 11 |
| | cytoskeleton | Q9NVD7 | PARVA | Alpha-Parvin | 100(4) | 100(6) | 100(2) | 12 |
| | reorganisation | P15311 | EZR | Ezrin | 100(7) | 100(4) | 100(3) | 13 |
| | | Q13308 | PTK7 | Inactive tyrosine-protein | 100(9) | 100(8) | 100(2) | 14 |
| | | | | kinase 7 | | | | |
| | | P11233 | RALA | Ras-related protein Ral-A | 100(3) | 100(2) | 100(2) | 15 |
| | | P35222 | CTNNB1 | Catenin beta-1 | 100(8) | 100(8) | 100(2) | 16 |
| | Cell-cell | P18206 | VCL | Vinculin | 100(17) | 100(26) | 100(20) | 17 |
| | junction | Q9Y490 | TLN1 | Talin-1 | 100(34) | 100(36) | 100(4) | 18 |
| | ATP activity | P05023 | ATP1A1 | Sodium/potassium- | 100(6) | 100(4) | 100(4) | 19 |
| | | | | transporting ATPase | | | | |
| | | | | subunit alpha-1 | | | | |
| | | P54709 | ATP1B3 | Sodium/potassium- | 100(3) | 100(1) | 100(2) | 20 |
| | | | | transporting ATPase | | | | |
| | | | | subunit beta-3 | | | | |
| | Biosynthetic | Q9HDC9 | APMAP | Adipocyte plasma | 97(1) | 99(1) | 100(1) | 21 |
| | processes | | | membrane-associated | | | | |
| | | | | protein | | | | |
| | Blood | Q9NZM1 | MYOF | Myoferlin | 100(9) | 100(8) | 100(4) | 22 |
| | circulation and | P13987 | CD59 | CD59 glycoprotein | 100(3) | 100(2) | 100(4) | 23 |
| | remodelling | P12821 | ACE | Angiotensin-converting | 99(2) | 100(1) | 100(2) | 24 |
| | | | | enzyme | | | | |
| | Transport | P23634 | ATP2B4 | Plasma membrane | 100(7) | 100(7) | 100(4) | 25 |
| | | | | calcium-transporting | | | | |
| | | | | ATPase 4 | | | | |
| | | P23526 | AHCY | Adenosylhomocysteinase | 100(4) | 100(6) | 100(6) | 26 |
| | | Q03135 | CAV1 | Caveolin | 100(1) | 99(1) | 100(1) | 27 |
| | | P27797 | CALR | Calreticulin | 100(5) | 100(5) | 100(4) | 28 |
| | | O00299 | CLIC1 | Chloride intracellular | 100(2) | 100(1) | 100(2) | 29 |
| | | | | channel protein 1 | | | | |
| | | P51148 | RAB5C | Ras-related protein | 100(2) | 100(1) | 100(1) | 30 |
| | | | | Rab-5C | | | | |
| | | O43493 | TGOLN2 | Trans-golgi network | 99(1) | 99(1) | 99(1) | 31 |
| | | | | protein 2, isoform CRA | | | | |
| | Cell | P15144 | ANPEP | Aminopeptidase N | 100(8) | 100(3) | 100(2) | 32 |
| | differentiation | P80723 | BASP1 | Brain acid soluble protein | 100(3) | 100(3) | 100(6) | 33 |
| | | | | 1 | | | | |
| | Cell | P04632 | CAPNS1 | Calpain small subunit 1 | 100(2) | 100(2) | 100(1) | 34 |
| | proliferation | | | | | | | |
| | Cell-cell | O14672 | ADAM10 | Disintegrin and | 100(7) | 100(4) | 100(2) | 35 |
| | signalling | | | metalloproteinase domain- | | | | |
| | | | | containing protein 10 | | | | |
| | | Q07075 | ENPEP | Glutamyl aminopeptidase | 100(4) | 100(1) | 99(1) | 36 |
| | Cellular | P46940 | IQGAP1 | IQGAP1 IQ motif | 100(19) | 100(21) | 100(15) | 37 |
| | response | | | containing GTPase | | | | |
| | | | | activating protein 1 | | | | |
| | | Q9UBI6 | GNG12 | Guanine nucleotide- | 100(2) | 99(1) | 100(1) | 38 |
| | | | | binding protein | | | | |
| | | | | G(I)/G(S)/G(O) subunit | | | | |
| | | | | gamma-12] | | | | |
| | | P61769 | B2M | Beta-2-microglobulin | 100(1) | 100(1) | 100(1) | 39 |
| | Establishment | P26038 | MSN | Moesin | 100(11) | 100(9) | 100(4) | 40 |
| | of cellular | | | | | | | |
| | polarity | | | | | | | |
| | Glycolysis | P06733 | ENO1 | Alpha-enolase | 100(7) | 100(8) | 100(6) | 41 |
| | Lipid particle | Q9P2B2 | PTGFRN | Prostaglandin F2 receptor | 100(5) | 100(6) | 100(3) | 42 |
| | organisation | | | negative regulator | | | | |
| | Metabolic | P08473 | MME | Neprilysin | 100(3) | 100(4) | 100(1) | 43 |
| | process | | | | | | | |
| | Protein folding | P07900 | HSP90AA1 | Heat shock protein HSP | 100(18) | 100(23) | 100(10) | 44 |
| | | | | 90-alpha | | | | |
| | | Q15084 | PDIA6 | Isoform 2 of Protein | 100(7) | 100(12) | 100(7) | 45 |
| | | | | disulfide-isomerase A6 | | | | |
| | Metabolic | Q07065 | CKAP4 | Cytoskeleton-associated | 100(3) | 100(2) | 100(5) | 46 |
| | | | | protein 4 | | | | |
| | processing | P14384 | CPM | Carboxypeptidase M | 100(2) | 100(5) | 100(2) | 47 |
| Cytoplasm | | P43686 | PSMC4 | 26S proteasome regulatory | 100% | 100% | 100% | 48 |
| | | | | subunit 6B | | | | |
| | | P25398 | RPS12 | 40S ribosomal protein S12 | 100% | 100% | 100% | 49 |
| | | P42677 | RPS27 | 40S ribosomal protein S27 | 100% | 100% | 100% | 50 |
| | | P23396 | RPS3 | 40S ribosomal protein S3 | 100% | 100% | 100% | 51 |
| | | P61247 | RPS3A | 40S ribosomal protein S3a | 100% | 100% | 100% | 52 |
| | | P62701 | RPS4X | 40S ribosomal protein S4, | 100% | 100% | 100% | 53 |
| | | | | X isoform | | | | |
| | | P61513 | RPL37 | 60S ribosomal protein L37 | 100% | 100% | 100% | 54 |
| | | P26373 | RPL13 | 60S ribosomal protein L13 | 100% | 100% | 100% | 55 |
| | | P40429 | RPL13A | 60S ribosomal protein | 100% | 100% | 100% | 56 |
| | | | | L13a | | | | |
| | | P39023 | RPL3 | 60S ribosomal protein L3 | 100% | 100% | 100% | 57 |
| | | P62917 | RPL8 | 60S ribosomal protein L8 | 100% | 100% | 100% | 58 |
| | | P62736 | ACTA2 | Actin, aortic smooth | 100% | 100% | 100% | 59 |
| | | | | muscle | | | | |
| | | O15143 | ARPC1B | Actin-related protein 2/3 | 100% | 100% | 100% | 60 |
| | | | | complex subunit 1B | | | | |
| | | O15144 | ARPC2 | Actin-related protein 2/3 | 100% | 100% | 100% | 61 |
| | | | | complex subunit 2 | | | | |
| | | P61158 | ACTR3 | Actin-related protein 3 | 100% | 100% | 100% | 62 |
| | | Q9ULA0 | DNPEP | Aspartyl aminopeptidase | 100% | 100% | 100% | 63 |
| | | P04040 | CAT | Catalase | 100% | 100% | 100% | 64 |
| | | Q15717 | ELAVL1 | ELAV-like protein 1 | 100% | 100% | 100% | 65 |
| | | P13639 | EEF2 | Elongation factor 2 | 100% | 100% | 100% | 66 |
| | | P38919 | EIF4A3 | Eukaryotic initiation | 100% | 100% | 100% | 67 |
| | | | | factor 4A-III | | | | |
| | | O00303 | EIF3F | Eukaryotic translation | 100% | 100% | 100% | 68 |
| | | | | initiation factor 3 subunit F | | | | |
| | | Q13347 | EIF3I | Eukaryotic translation | 100% | 100% | 100% | 69 |
| | | | | initiation factor 3 subunit I | | | | |
| | | P55060 | CSE1L | Exportin-2 | 100% | 100% | 100% | 70 |
| | | P04792 | HSPB1 | Heat shock protein beta-1 | 100% | 100% | 100% | 71 |
| | | O94788-2 | ALDH1A2 | Aldehyde dehydrogenase | 100% | 100% | 100% | 72 |
| | | | | 1 family member 2A | | | | |
| | | Q86XR7-2 | TICAM2 | Toll like receptor adaptor | 100% | 100% | 100% | 73 |
| | | | | molecule 2 | | | | |
| | | Q16851-2 | UGP2 | UTP-glucose-1-phosphate | 100% | 100% | 100% | 74 |
| | | | | uridylyltransferase | | | | |
| | | P40926 | MDH2 | Malate dehydrogenase | 100% | 100% | 100% | 75 |
| | | Q96IJ6 | GMPPA | Mannose-1-phosphate | 100% | 100% | 100% | 76 |
| | | | | guanyltransferase alpha | | | | |
| | | Q06830 | PRDX1 | Peroxiredoxin-1 | 100% | 100% | 100% | 77 |
| | | Q13162 | PRDX4 | Peroxiredoxin-4 | 100% | 100% | 100% | 78 |
| | | P30041 | PRDX6 | Peroxiredoxin-6 | 100% | 100% | 100% | 79 |
| | | P00558 | PGK1 | Phosphoglycerate kinase 1 | 100% | 100% | 100% | 80 |
| | | P25787 | PSMA2 | Proteasome subunit alpha | 100% | 100% | 100% | 81 |
| | | | | type-2 | | | | |
| | | P14618 | PKM | Pyruvate kinase PKM | 100% | 100% | 100% | 82 |
| | | P52565 | ARHGDIA | Rho GDP-dissociation | 100% | 100% | 100% | 83 |
| | | | | inhibitor 1 | | | | |
| | | Q07960 | ARHGAP1 | Rho GTPase-activating | 100% | 100% | 100% | 84 |
| | | | | protein 1 | | | | |
| | | EAW72088 | RPL5 | Ribosomal protein L5, | 100% | 100% | 100% | 85 |
| | | | | isoform CRA_b | | | | |
| | | Q9UHD8 | SEPT9 | Septin-9 | 100% | 100% | 100% | 86 |
| | | P49458 | SRP9 | Signal recognition particle | 100% | 100% | 100% | 87 |
| | | | | 9 kDa protein | | | | |
| | | P62314 | SNRPD1 | Small nuclear | 100% | 100% | 100% | 88 |
| | | | | ribonucleoprotein Sm D1 | | | | |
| | | Q99832 | CCT7 | T-complex protein 1 | 100% | 100% | 100% | 89 |
| | | | | subunit eta | | | | |
| | | P10599 | TXN | Thioredoxin | 100% | 100% | 100% | 90 |
| | | P37837 | TALDO1 | Transaldolase | 100% | 100% | 100% | 91 |
| | | P55072 | VCP | Transitional endoplasmic | 100% | 100% | 100% | 92 |
| | | | | reticulum ATPase | | | | |
| | | P22314 | UBA1 | Ubiquitin-like modifier- | 100% | 100% | 100% | 93 |
| | | | | activating enzyme 1 | | | | |
| | | P35998 | PSMC2 | 26S protease regulatory | 100% | 100% | 100% | 94 |
| | | | | subunit 7 | | | | |
| | | P46783 | RPS10 | 40S ribosomal protein S10 | 100% | 100% | 100% | 95 |
| | | P62269 | RPS18 | 40S ribosomal protein S18 | 100% | 100% | 100% | 96 |
| | | P15880 | RPS2 | 40S ribosomal protein S2 | 100% | 100% | 100% | 97 |
| | | P60866 | RPS20 | 40S ribosomal protein S20 | 100% | 100% | 100% | 98 |
| | | P62906 | RPL10A | 60S ribosomal protein L10a | 100% | 100% | 100% | 99 |
| | | Q01813 | PFKP | ATP-dependent 6- | 100% | 100% | 100% | 100 |
| | | | | phosphofructokinase, | | | | |
| | | | | platelet type | | | | |
| | | P59998 | ARPC4 | Actin-related protein 2/3 | 100% | 100% | 100% | 101 |
| | | | | complex subunit 4 | | | | |
| | | Q01518 | CAP1 | Adenylyl cyclase- | 100% | 100% | 100% | 102 |
| | | | | associated protein 1 | | | | |
| | | P11766 | ADH5 | Alcohol dehydrogenase | 100% | 100% | 100% | 103 |
| | | | | class-3 | | | | |
| | | O43707 | ACTN4 | Alpha-actinin-4 | 100% | 100% | 100% | 104 |
| | | P48444 | ARCN1 | Archain 1, isoform CRA | 100% | 100% | 100% | 105 |
| | | Q13867 | BLMH | Bleomycin hydrolase | 100% | 100% | 100% | 106 |
| | | P35606 | COPB2 | Coatomer protein | 100% | 100% | 100% | 107 |
| | | | | complex, subunit beta 2 | | | | |
| | | Q86VP6 | CAND1 | Cullin-associated | 100% | 100% | 100% | 108 |
| | | | | NEDD8-dissociated | | | | |
| | | | | protein 1 | | | | |
| | | Q14204 | DYNC1H1 | Cytoplasmic dynein 1 | 100% | 100% | 100% | 109 |
| | | | | heavy chain 1 | | | | |
| | | Q13409 | DYNC1I2 | Cytoplasmic dynein 1 | 100% | 100% | 100% | 110 |
| | | | | intermediate chain 2 | | | | |
| | | Q16555 | DPYSL2 | Dihydropyrimidinase- | 100% | 100% | 100% | 111 |
| | | | | related protein 2 | | | | |
| | | Q16531 | DDB1 | DNA damage-binding | 100% | 100% | 100% | 112 |
| | | | | protein 1 | | | | |
| | | Q16643 | DBN1 | Drebrin | 100% | 100% | 100% | 113 |
| | | Q14203 | DCTN1 | Dynactin subunit 1 | 100% | 100% | 100% | 114 |
| | | P68103 | EEF1A1 | Elongation factor 1-alpha 1 | 100% | 100% | 100% | 115 |
| | | P62495 | ETF1 | Eukaryotic peptide chain | 100% | 100% | 100% | 116 |
| | | | | release factor subunit 1 | | | | |
| | | Q14152 | EIF3A | Eukaryotic translation | 100% | 100% | 100% | 117 |
| | | | | initiation factor 3 subunit A | | | | |
| | | P55884 | EIF3B | Eukaryotic translation | 100% | 100% | 100% | 118 |
| | | | | initiation factor 3 subunit B | | | | |
| | | Q99613 | EIF3C | Eukaryotic translation | 100% | 100% | 100% | 119 |
| | | | | initiation factor 3 subunit C | | | | |
| | | P52907 | CAPZA1 | F-actin-capping protein | 100% | 100% | 100% | 120 |
| | | | | subunit alpha-1 | | | | |
| | | P49327 | FASN | Fatty acid synthase | 100% | 100% | 100% | 121 |
| | | P21333 | FLNA | Filamin-a | 100% | 100% | 100% | 122 |
| | | P04075 | ALDOA | Fructose-bisphosphate | 100% | 100% | 100% | 123 |
| | | | | aldolase A | | | | |
| | | P17931 | LGALS3 | Galectin-3 | 100% | 100% | 100% | 124 |
| | | Q08380 | LGALS3BP | Galectin-3-binding protein | 100% | 100% | 100% | 125 |
| | | P06744 | GPI | Glucose-6-phosphate | 100% | 100% | 100% | 126 |
| | | | | isomerase | | | | |
| | | P09211 | GSTP1 | Glutathione S-transferase P | 100% | 100% | 100% | 127 |
| | | P04406 | GAPDH | Glyceraldehyde-3- | 100% | 100% | 100% | 128 |
| | | | | phosphate dehydrogenase | | | | |
| | | P11142 | HSPA8 | Heat shock cognate 71 | 100% | 100% | 100% | 129 |
| | | | | kDa protein | | | | |
| | | P35579 | MYH9 | Myosin-9 | 100% | 100% | 100% | 130 |
| | | P67809 | YBX1 | Nuclease-sensitive | 100% | 100% | 97% | 131 |
| | | | | element-binding protein 1 | | | | |
| | | P19338 | NCL | Nucleolin | 100% | 100% | 93% | 132 |
| | | P13796 | LCP1 | Plastin-2 | 100% | 99% | 100% | 133 |
| | | P11940 | PABPC1 | Polyadenylate-binding | 100% | 99% | 100% | 134 |
| | | | | protein 1 | | | | |
| | | P26599 | PTBP1 | Polypyrimidine tract- | 100% | 0 | 100% | 135 |
| | | | | binding protein 1 | | | | |
| | | P07737 | PFN1 | Profilin-1 | 100% | 0 | 100% | 136 |
| | | Q8WUM4 | PDCD6IP | Programmed cell death 6- | 100% | 0 | 99% | 137 |
| | | | | interacting protein | | | | |
| | | P25786 | PSMA1 | Proteasome subunit alpha | 99% | 100% | 100% | 138 |
| | | | | type-1 | | | | |
| | | P25789 | PSMA4 | Proteasome subunit alpha | 99% | 100% | 100% | 139 |
| | | | | type-4 | | | | |
| | | O14818 | PSMA7 | Proteasome subunit alpha | 99% | 100% | 100% | 140 |
| | | | | type-7 | | | | |
| | | P20618 | PSMB1 | Proteasome subunit beta | 99% | 100% | 100% | 141 |
| | | | | type-1 | | | | |
| | | P40306 | PSMB10 | Proteasome subunit beta | 99% | 100% | 100% | 142 |
| | | | | type-10 | | | | |
| | | P49720 | PSMB3 | Proteasome subunit beta | 99% | 100% | 100% | 143 |
| | | | | type-3 | | | | |
| | | P28070 | PSMB4 | Proteasome subunit beta | 99% | 100% | 100% | 144 |
| | | | | type-4 | | | | |
| | | P28074 | PSMB5 | Proteasome subunit beta | 99% | 100% | 100% | 145 |
| | | | | type-5 | | | | |
| | | Q99497 | PARK7 | Protein DJ-1 | 98% | 100% | 100% | 146 |
| | | P31949 | S100A11 | Protein S100-A11 | 96% | 100% | 100% | 147 |
| | | O94979 | SEC31A | Protein transport protein | 96% | 100% | 100% | 148 |
| | | | | Sec31A | | | | |
| | | Q9HCE1 | MOV10 | Putative helicase MOV-10 | 96% | 100% | 100% | 149 |
| | | Q9BRX8 | PRXL2A | Redox-regulatory protein | 95% | 100% | 100% | 150 |
| | | | | FAM213A | | | | |
| | | P00352 | ALDH1A1 | Retinal dehydrogenase 1 | 93% | 100% | 100% | 151 |
| | | P13489 | RNH1 | Ribonuclease inhibitor | 93% | 100% | 100% | 152 |
| | | Q16181 | SEPT7 | Septin 7 | 0 | 100% | 100% | 153 |
| | | Q01130 | SFRS2 | Serine/arginine-rich- | 0 | 100% | 100% | 154 |
| | | | | splicing factor 2 | | | | |
| | | P62306 | SNRPF | Small nuclear | 0 | 100% | 100% | 155 |
| | | | | ribonucleoprotein F | | | | |
| | | P62318 | SNRPD3 | Small nuclear | 0 | 100% | 100% | 156 |
| | | | | ribonucleoprotein Sm D3 | | | | |
| | | P30626 | SRI | Sorcin | 0 | 100% | 100% | 157 |
| | | Q01082 | SPTBN1 | Spectrin beta chain, non- | 0 | 100% | 100% | 158 |
| | | | | erythrocytic 1 | | | | |
| | | Q15393 | SF3B3 | Splicing factor 3B subunit 3 | 0 | 100% | 100% | 159 |
| | | Q7KZF4 | SND1 | Staphylococcal nuclease | 0 | 100% | 100% | 160 |
| | | | | domain-containing protein 1 | | | | |
| | | Q99536 | VAT1 | Synaptic vesicle | 0 | 100% | 100% | 161 |
| | | | | membrane protein VAT-1 | | | | |
| | | | | homolog | | | | |
| | | P17987 | TCP1 | T-complex protein 1 | 0 | 100% | 100% | 162 |
| | | | | subunit alpha | | | | |
| | | P78371 | CCT2 | T-complex protein 1 | 0 | 100% | 100% | 163 |
| | | | | subunit beta | | | | |
| | | P50991 | CCT4 | T-complex protein 1 | 0 | 100% | 100% | 164 |
| | | | | subunit delta | | | | |
| | | P48643 | CCT5 | T-complex protein 1 | 0 | 100% | 100% | 165 |
| | | | | subunit epsilon | | | | |
| | | P50990 | CCT8 | T-complex protein 1 | 0 | 100% | 100% | 166 |
| | | | | subunit theta | | | | |
| | | Q99598 | TSN | Translin | 0 | 100% | 100% | 167 |
| | | P06753 | TPM3 | Tropomyosin alpha-3 | 0 | 100% | 100% | 168 |
| | | | | chain | | | | |
| | | P67936 | TPM4 | Tropomyosin alpha-4 | 0 | 100% | 100% | 169 |
| | | | | chain | | | | |
| | | P68366 | TUBA4A | Tubulin alpha-4A chain | 0 | 100% | 100% | 170 |
| | | Q12792 | TWF1 | Twinfilin-1 | 0 | 100% | 100% | 171 |
| | | P62979 | RPS27A | Ubiquitin-40S ribosomal | 0 | 100% | 100% | 172 |
| | | | | protein S27a | | | | |
| | | O75083 | WDR1 | WD repeat-containing | 0 | 100% | 100% | 173 |
| | | | | protein 1 | | | | |
| | | P16989 | YBX3 | Y-box-binding protein 3 | 0 | 100% | 100% | 174 |
| Nucleus | | P46776 | RPL27A | 60S ribosomal protein | 100% | 100% | 100% | 175 |
| | | | | L27a | | | | |
| | | O75367 | H2AFY | Core histone macro- | 100% | 100% | 100% | 176 |
| | | | | H2A.1 | | | | |
| | | P24534 | EEF1B2 | Elongation factor 1-beta | 100% | 100% | 100% | 177 |
| | | P29692 | EEF1D | Elongation factor 1-delta | 100% | 100% | 100% | 178 |
| | | P60228 | EIF3E | Eukaryotic translation | 100% | 100% | 100% | 179 |
| | | | | initiation factor 3 subunit E | | | | |
| | | P35573 | AGL | Glycogen debranching | 100% | 100% | 100% | 180 |
| | | | | enzyme | | | | |
| | | O60832 | DKC1 | H/ACA ribonucleoprotein | 100% | 100% | 100% | 181 |
| | | | | complex subunit DKC1 | | | | |
| | | P46087 | NOP2 | Isoform 2 of Putative | 100% | 100% | 100% | 182 |
| | | | | ribosomal RNA | | | | |
| | | | | methyltransferase NOP2 | | | | |
| | | P55769 | SNU13 | NHP2-like protein 1 | 100% | 100% | 100% | 183 |
| | | P55209 | NAP1L1 | Nucleosome assembly | 100% | 100% | 100% | 184 |
| | | | | protein 1-like 1 | | | | |
| | | O75340 | PDCD6 | Programmed cell death | 100% | 100% | 100% | 185 |
| | | | | protein 6 | | | | |
| | | Q52LJ0 | FAM98B | Protein FAM98B | 100% | 100% | 100% | 186 |
| | | Q13838 | DDX39B | Spliceosome RNA | 100% | 100% | 100% | 187 |
| | | | | helicase DDX39B | | | | |
| | | P06454 | PTMA | Thymosin alpha-1 | 100% | 100% | 100% | 188 |
| | | P13010 | XRCC5 | X-ray repair cross- | 100% | 100% | 100% | 189 |
| | | | | complementing protein 5 | | | | |
| | | P39019 | RPS19 | 40S ribosomal protein S19 | 100% | 100% | 100% | 190 |
| | | P42766 | RPL35 | 60S ribosomal protein L35 | 100% | 100% | 100% | 191 |
| | | Q08211 | DHX9 | ATP-dependent RNA | 100% | 100% | 100% | 192 |
| | | | | helicase A | | | | |
| | | P27695 | APEX1 | DNA-(apurinic or | 100% | 100% | 100% | 193 |
| | | | | apyrimidinic site) lyase | | | | |
| | | P04843 | RPN1 | Dolichyl- | 100% | 100% | 100% | 194 |
| | | | | diphosphooligosaccharide-- | | | | |
| | | | | protein | | | | |
| | | | | glycosyltransferase | | | | |
| | | | | subunit 1 | | | | |
| | | Q9UBQ5 | EIF3K | Eukaryotic translation | 100% | 100% | 100% | 195 |
| | | | | initiation factor 3 subunit K | | | | |
| | | Q9Y262 | EIF3L | Eukaryotic translation | 100% | 100% | 100% | 196 |
| | | | | initiation factor 3 subunit L | | | | |
| | | Q9NY12 | GAR1 | GAR1 H/ACA | 100% | 100% | 100% | 197 |
| | | | | ribonucleoprotein | | | | |
| | | | | complex subunit 1 | | | | |
| | | Q5SSJ5 | HP1BP3 | Heterochromatin protein | 100% | 100% | 100% | 198 |
| | | | | 1-binding protein 3 | | | | |
| | | P52597 | HNRNPF | Heterogeneous nuclear | 100% | 100% | 100% | 199 |
| | | | | ribonucleoprotein F | | | | |
| | | P52272 | HNRNPM | Heterogeneous nuclear | 100% | 100% | 100% | 200 |
| | | | | ribonucleoprotein M | | | | |
| | | Q1KMD3 | HNRNPUL2 | Heterogeneous nuclear | 100% | 100% | 100% | 201 |
| | | | | ribonucleoprotein U-like | | | | |
| | | | | protein 2 | | | | |
| | | P16401 | HIST1H1B | Histone H1.5 | 100% | 100% | 100% | 202 |
| | | Q09666 | AHNAK | Neuroblast differentiation- | 100% | 100% | 100% | 203 |
| | | | | associated protein | | | | |
| | | | | AHNAK | | | | |
| | | P43490 | NAMPT | Nicotinamide | 100% | 100% | 99% | 204 |
| | | | | phosphoribosyltransferase | | | | |
| | | O00567 | NOP56 | Nucleolar protein 56 | 100% | 100% | 93% | 205 |
| | | P06748 | NPM1 | Nucleophosmin | 100% | 100% | 91% | 206 |
| | | P02545 | LMNA | Prelamin-A/C | 100% | 0 | 100% | 207 |
| | | P27694 | RPA1 | Replication protein A 70 | 94% | 100% | 100% | 208 |
| | | | | kDa DNA-binding subunit | | | | |
| | | Q01130 | SRSF2 | Serine/arginine-rich- | 0 | 100% | 100% | 209 |
| | | | | splicing factor 2 | | | | |
| | | P84103 | SRSF3 | Serine/arginine-rich- | 0 | 100% | 100% | 210 |
| | | | | splicing factor 3 | | | | |
| | | Q16629 | SRSF7 | Serine/arginine-rich- | 0 | 100% | 100% | 211 |
| | | | | splicing factor 7 | | | | |
| | | P63162 | SNRPN | Small nuclear | 0 | 100% | 100% | 212 |
| | | | | ribonucleoprotein- | | | | |
| | | | | associated protein N | | | | |
| | | Q92522 | H1FX | Histone H1x | 0 | 100% | 100% | 213 |
| | | P26368 | U2AF2 | Splicing factor U2AF 65 | 0 | 100% | 100% | 214 |
| | | | | kDa subunit | | | | |
| | | P12956 | XRCC6 | X-ray repair cross- | 0 | 100% | 100% | 215 |
| | | | | complementing protein 6 | | | | |
| Other | | P11021 | HSPA5 | 78 kDa glucose-regulated | 100% | 100% | 100% | 216 |
| | | | | protein | | | | |
| | | P27824 | CANX | Calnexin | 100% | 100% | 100% | 217 |
| | | P07339 | CTSD | Cathepsin D | 100% | 100% | 100% | 218 |
| | | Q9Y2Q3 | GSTK1 | Glutathione S-transferase | 100% | 100% | 100% | 219 |
| | | | | kappa 1 | | | | |
| | | Q92896 | GLG1 | Golgi apparatus protein 1 | 100% | 100% | 100% | 220 |
| | | P11047 | LAMC1 | Laminin subunit gamma-1 | 100% | 100% | 100% | 221 |
| | | Q9P2E9 | RRBP1 | RRBP1 protein | 100% | 100% | 100% | 222 |
| | | P22314 | UBA1 | Ubiquitin-like modifier- | 100% | 100% | 100% | 223 |
| | | | | activating enzyme 1 | | | | |
| | | Q99714 | HSD17B10 | 3-hydroxyacyl-CoA | 100% | 100% | 100% | 224 |
| | | | | dehydrogenase type-2 | | | | |
| | | P55084 | HADHB | 3-ketoacyl-CoA thiolase | 100% | 100% | 100% | 225 |
| | | P18085 | ARF4 | ADP-ribosylation factor 4 | 100% | 100% | 100% | 226 |
| | | P06576 | ATP5F1B | ATP synthase subunit | 100% | 100% | 100% | 227 |
| | | | | beta, mitochondrial | | | | |
| | | Q13938 | CAPS | Calcyphosin | 100% | 100% | 100% | 228 |
| | | P12111 | COL6A3 | Collagen alpha-3(VI) | 100% | 100% | 100% | 229 |
| | | | | chain | | | | |
| | | Pl2277 | CKB | Creatine kinase B-type | 100% | 100% | 100% | 230 |
| | | P30040 | ERP29 | Endoplasmic reticulum | 100% | 100% | 100% | 231 |
| | | | | resident protein 29 | | | | |
| | | Q9BS26 | ERP44 | Endoplasmic reticulum | 100% | 100% | 100% | 232 |
| | | | | resident protein 44 | | | | |
| | | P09382 | LGALS1 | Galectin-1 | 100% | 100% | 100% | 233 |
| | | P22749 | GNLY | Granulysin | 100% | 100% | 100% | 234 |
| | | P34932 | HSPA4 | Heat shock 70 kDa protein 4 | 100% | 100% | 100% | 235 |
| | | Q9Y4L1 | HYOU1 | Hypoxia up-regulated | 100% | 100% | 100% | 236 |
| | | | | protein 1 | | | | |
| | | P00387 | CYB5R3 | NADH-cytochrome b5 | 100% | 100% | 100% | 237 |
| | | | | reductase 3 | | | | |
| | | P14543 | NID1 | Nidogen-1 | 100% | 100% | 99% | 238 |
| | | P23284 | PPIB | Peptidyl-prolyl cis-trans | 100% | 100% | 0 | 239 |
| | | | | isomerase B | | | | |
| | | P07237 | P4HB | Protein disulfide- | 99% | 100% | 100% | 240 |
| | | | | isomerase | | | | |
| | | P13667 | PDIA4 | Protein disulfide- | 99% | 100% | 99% | 241 |
| | | | | isomerase A4 | | | | |
| | | O60493 | SNX3 | Sorting nexin-3 | 0 | 100% | 100% | 242 |
| | | O60635 | TSPAN1 | Tetraspanin-1 | 0 | 100% | 100% | 243 |
| | | P40939 | HADHA | Trifunctional enzyme | 0 | 100% | 100% | 244 |
| | | | | subunit alpha | | | | |
| | | P07355 | ANXA2 | Annexin A2 | 100% | 100% | 100% | 245 |
| | | Q9UBG0 | MRC2 | C-type mannose receptor 2 | 100% | 100% | 100% | 246 |
| | | P62888 | RPL30 | 60S ribosomal protein L30 | 100% | 100% | 100% | 247 |
| | | Q13561 | DCTN2 | Dynactin subunit 2 | 100% | 100% | 100% | 248 |
| | | Q5T4S7 | UBR4 | E3 ubiquitin-protein ligase | 100% | 100% | 100% | 249 |
| | | | | UBR4 | | | | |
| | | Q00796 | SORD | Sorbitol dehydrogenase | 0 | 100% | 100% | 250 |

### Example 3: PCX is Localized to the Apical Membrane of Epithelial and Endothelial Cells in the Human Endometrium and is Down-Regulated Specifically in the Luminal Epithelium Coinciding with Receptivity Establishment

The cellular localization of PCX in the human endometrium across the menstrual cycle was examined by immunohistochemistry, as described in Example 1.

All 3 PCX antibodies detected a similar pattern of staining. In the proliferative phase, PCX was localized strongly to the apical surface of both the luminal and glandular epithelial cells (LE and GE respectively), as well as of endothelial cells in blood vessels (BV). The stroma showed no/below detection. This pattern persisted more or less to the early secretory phase, after which drastic differences emerged, especially in LE. In the mid-secretary phase, while PCX staining was still strong in both GE and BV, it was almost non-detectable in LE. In the late-secretory phase, whilst LE continued to be with minimal PCX, GE displayed fainter PCX staining compared to earlier phases.

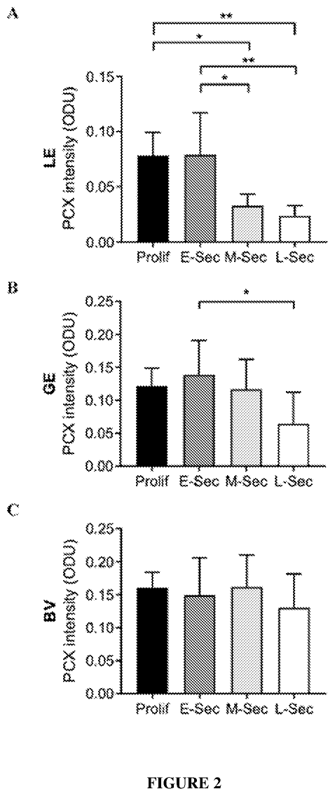

The PCX staining in LE, GE and BV across the cycle was quantified (FIG. 2A-C). As shown in FIG. 2, LE showed the most dramatic changes with cycle progression. PCX in LE was highest in the proliferative phase, but reduced profoundly and specifically from the mid-secretory phase, coinciding with the establishment of receptivity. In contrast, PCX in GE was variable and did not show significant reductions until the late-secretary phase. PCX in BV did not show significant cycle-dependent changes.

### Example 4: PCX is Enhanced by Estrogen and Reduced by Progesterone in Primary HEECs In Vitro

As estrogen (E) and progesterone (P) drive endometrial proliferation and differentiation respectively, the impact of these hormones on PCX in primary HEECs was determined.

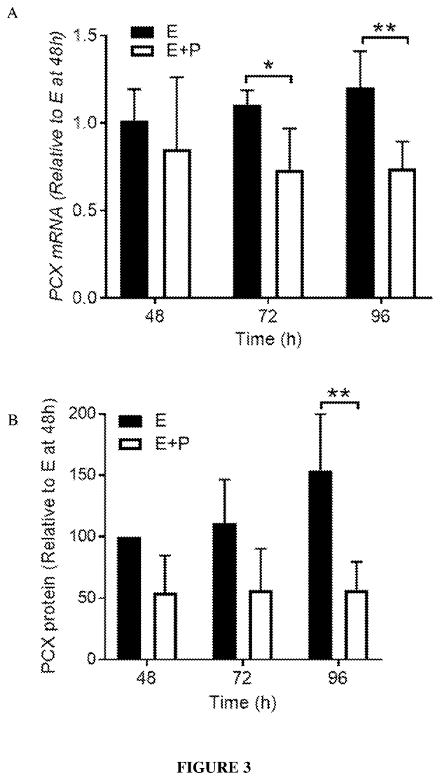

Primary HEECs from the proliferative phase (as for the proteomic study) were isolated and treated with E alone (to mimic the proliferative phase) or P following E priming (E+P, to mimic the secretory phase) for 48, 72 and 96 h respectively. Real-time RT-PCR analysis showed that PCX mRNA was gradually but subtly increased by E but reduced overtime by E+P (FIG. 3A), although the time-dependent changes were not statistically significantly neither for E nor for E+P. However, PCX mRNA was lower in cells treated with E+P than E alone significantly at 72 h, and highly significantly at 96 h (FIG. 3A). Western blot analysis showed a similar pattern of PCX protein changes albeit the difference between E vs E+P was significant only at 96 h (FIG. 3B).

To further validate this finding, HEECs treated with E or E+P for 96 h were analyzed by immunofluorescence. Cells treated with E showed strong PCX staining, whilst those treated with E+P displayed much reduced levels of PCX. Collectively, these results are consistent with E promoting whereas P reducing PCX in primary HEECs. However, PCX changes in isolated cells were not as drastic as those observed in LE in the endometrial tissue, very likely because primary cells were of a mixture of LE and GE origin (further subtype purification is not possible due to the lack of markers). Nevertheless, these results support the notion that P reduces PCX in endometrial epithelial cells.

### Example 5: PCX Knockdown Increases Whereas Overexpression Decreases Ishikawa Cell Adhesiveness

The unique expression pattern and hormonal regulation of PCX prompted investigation into whether PCX influences epithelial receptivity to embryo implantation. Due to the scarcity of primary HEECs, Ishikawa cells were employed for functional studies. PCX expression levels in Ishikawa cells were altered and their adhesiveness to fibronectin determined.

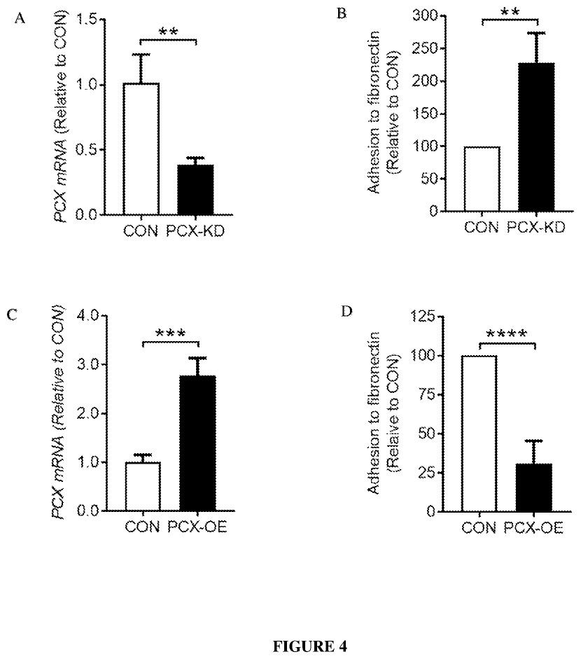

PCX was transiently knocked down (KD) in Ishikawa cells by siRNA. Real-time RT-PCR analysis showed a 60% reduction of PCX mRNA in PCX-KD compared to control (CON) cells (FIG. 4A). Western blot analysis further confirmed this knockdown. When tested for adhesion to fibronectin, PCX-KD cells were 2.5 times more adhesive than the control (FIG. 4B), suggesting that reducing PCX increased their adhesiveness.

Following this, PCX was overexpressed (OE) in Ishikawa cells. The full length human PCX was stably transfected into Ishikawa cells, and PCX overexpression was confirmed by RT-PCR (FIG. 4C) and western blot. The PCX-OE cells expressed 2.8 times of PCX than the control cells. These PCX-OE cells were 75% less adhesive than the control to fibronectin (FIG. 4D). Collectively, these results suggest an inverse correlation between the level of PCX expression and Ishikawa cell adhesiveness.

### Example 6: PCX Overexpression Reduces Ishikawa Cell Receptivity to Trophoblast Spheroid Attachment

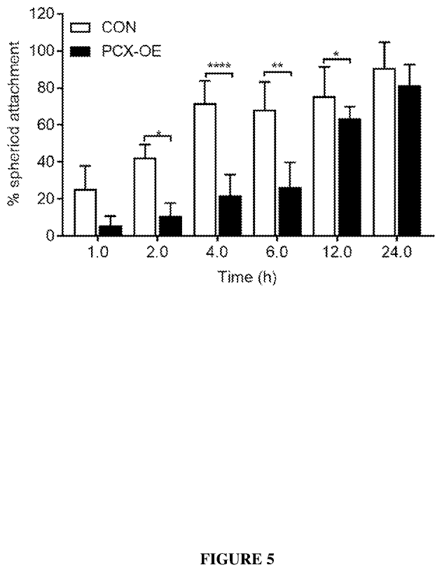

The impact of PCX-OE on Ishikawa receptivity to embryo attachment was examined using an in vitro model (Heng et al., 2015), in which a monolayer of Ishikawa cells mimics the endometrial luminal epithelium, and spheroids (˜100 μm) made of primary human trophoblasts mimics blastocysts. Equal numbers of spheroids were co-cultured on top of the Ishikawa monolayer and stable spheroid attachment was assessed over 24 h (FIG. 5). For the control monolayer, 25% of the added spheroids attached within 1 h, 42% within 2 h and 72% attached within 4 h. Thereafter the attachment increased slowly overtime, reaching 76% by 12 h and a maximal of 91% by 24 h. However, as shown in FIG. 5, the PCX-OE monolayer showed very different attachment dynamics. Only 6% of spheroids attached within 1 h and 11% within 2 h; the attachment slowly increased to 22% by 4 h and 27% by 6 h. Even at 12 h, spheroid attachment to PCX-OE monolayer (64%) was still significantly lower than the control (76%). It was only by 24 h that the PCX-OE, reaching a maximal attachment rate of 82%, did not significantly differ from the control. These results suggest that PCX reduced Ishikawa cell receptivity to trophoblast spheroid attachment, and it slowed down the process of attachment.

### Example 7: PCX Overexpression Impedes Invasion of Trophoblast Spheroids Through the Ishikawa Monolayer

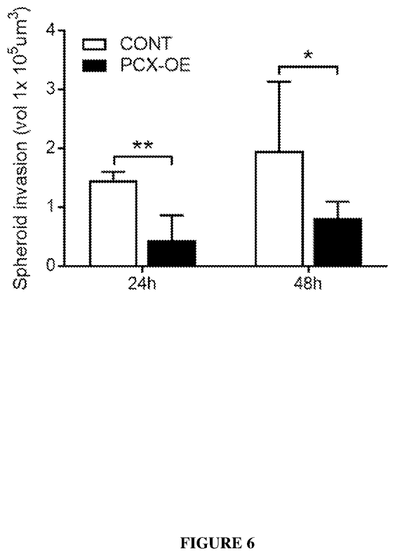

In the human, implantation requires the embryo to attach to the luminal epithelium then traverse between epithelial cells to move to the stroma. To investigate whether PCX influences the traversing process of trophoblast spheroids through the Ishikawa monolayer, we labelled trophoblast spheroids and Ishikawa cells with different dyes, cultured Ishikawa cells on a layer of matrix to form a monolayer, and then co-cultured the spheroids on top for 24 h and 48 h respectively. The position of trophoblast spheroids within the Ishikawa monolayer was examined by confocal z-stack scanning microscopy. By 24 h, spheroid invasion was clearly visible for the control monolayer, however, the process just started for the PCX-OE monolayer. By 48 h, all spheroids penetrated the monolayer, but the degree of penetration was still visibly less for the PCX-OE than control cells. The volume of spheroids present beneath the Ishikawa monolayer was quantified as a measurement of invasion (FIG. 6). The average spheroid volume beneath the PCX-OE monolayer was 30% (highly significant) and 40% (significant) of that of the control at 24 h and 48 h respectively. These data suggest that PCX-OE rendered the Ishikawa monolayer more difficult for trophoblast spheroids to traverse.

### Example 8: PCX Overexpression in Ishikawa Cells Also Hinders Attachment and Invasion of Human Embryos

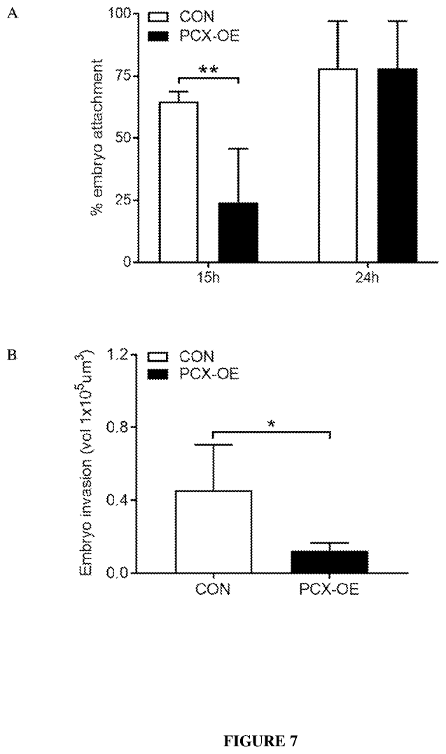

The in vitro attachment and invasion assays were repeated using human embryos in place of trophoblast spheroids. Human blastocysts were co-cultured on top of control and PCX-OE Ishikawa cell monolayers, and stable attachment was assessed at 15 h and 24 h respectively (FIG. 7A). At 15 h, 65% of blastocysts added to the control monolayer attached, whereas only 25% attached to the PCX-OE monolayer. By 24 h, however, the attachment rate reached 78% for both monolayers. This data suggests that PCX in Ishikawa monolayer again reduced the speed of embryo attachment, consistent with the observation made with trophoblast spheroids.

Embryo invasion through the Ishikawa monolayer was then assessed. Dye-labelled blastocysts were co-cultured on top of dye-labelled Ishikawa monolayer for 24 h, and the position of the embryo within the monolayer was examined by confocal imaging. Embryo invasion was visually less for the PCX-OE than control monolayer. The quantified volume of embryos that penetrated through the PCX-OE monolayer was significantly lower than that of the control (FIG. 7B). Embryo invasion at 48 h was also assessed however, all embryos had collapsed by that point and no data was available. These results suggest that PCX also hindered embryo traversing through the Ishikawa monolayer, again consistent with the observation made with trophoblast spheroids.

### Example 9: PCX Overexpression Down-Regulates Genes Required for Cell Adhesion and Implantation but Up-Regulates Those Controlling Epithelial Barrier Functions

### RNAseq Analysis of Control and PCX-OE Ishikawa Cells

To understand how PCX renders Ishikawa cells to be less receptive to embryo attachment and invasion, total mRNA transcription of control and PCX-OE Ishikawa cells was compared by RNAseq. Expression of 15,103 genes was detected, and the two cell types clustered into two distinctive groups by an unsupervised clustering analysis (data not shown). A total of 940 genes were found to be expressed significantly different between the two groups [p<0.01, Log(2)FC>2 or <−2], with 659 down-regulated and 281 up-regulated in PCX-OE compared to the control (Table 3).

TABLE 3
Genes that were expressed significantly differently
between PCX-OE and control Ishikawa cells.

| | | | Levels in PCX- | |
|---|---|---|---|---|
| | | | OE compared to | |
| Gene ID | Gene Name | EntrezGene ID | control cells | number |
| ENSG00000123243 | ITIH5 | 80760 | Up-regulated | 1 |
| ENSG00000170370 | EMX2 | 2018 | Up-regulated | 2 |
| ENSG00000106541 | AGR2 | 10551 | Up-regulated | 3 |
| ENSG00000102390 | PBDC1 | 51260 | Up-regulated | 4 |
| ENSG00000144057 | ST6GAL2 | 84620 | Up-regulated | 5 |
| ENSG00000132698 | RAB25 | 57111 | Up-regulated | 6 |
| ENSG00000196533 | NA | NA | Up-regulated | 7 |
| ENSG00000189334 | S100A14 | 57402 | Up-regulated | 8 |
| ENSG00000187156 | NA | NA | Up-regulated | 9 |
| ENSG00000167741 | GGT6 | 124975 | Up-regulated | 10 |
| ENSG00000217236 | SP9 | 100131390 | Up-regulated | 11 |
| ENSG00000143768 | LEFTY2 | 7044 | Up-regulated | 12 |
| ENSG00000094755 | GABRP | 2568 | Up-regulated | 13 |
| ENSG00000143416 | SELENBP1 | 8991 | Up-regulated | 14 |
| ENSG00000197083 | ZNF300P1 | NA | Up-regulated | 15 |
| ENSG00000118322 | ATP10B | 23120 | Up-regulated | 16 |
| ENSG00000229847 | EMX2OS | NA | Up-regulated | 17 |
| ENSG00000113209 | PCDHB5 | 26167 | Up-regulated | 18 |
| ENSG00000112494 | UNC93A | 54346 | Up-regulated | 19 |
| ENSG00000154764 | WNT7A | 7476 | Up-regulated | 20 |
| ENSG00000064655 | EYA2 | 2139 | Up-regulated | 21 |
| ENSG00000154451 | GBP5 | 115362 | Up-regulated | 22 |
| ENSG00000070526 | ST6GALNAC1 | 55808 | Up-regulated | 23 |
| ENSG00000083307 | GRHL2 | 79977 | Up-regulated | 24 |
| ENSG00000240754 | NA | NA | Up-regulated | 25 |
| ENSG00000180353 | HCLS1 | 3059 | Up-regulated | 26 |
| ENSG00000162949 | CAPN13 | 92291 | Up-regulated | 27 |
| ENSG00000204983 | PRSS1 | 5644 | Up-regulated | 28 |
| ENSG00000250591 | PRSS3P1 | NA | Up-regulated | 29 |
| ENSG00000120457 | KCNJ5 | 3762 | Up-regulated | 30 |
| ENSG00000187621 | TCL6 | NA | Up-regulated | 31 |
| ENSG00000155495 | MAGEC1 | 9947 | Up-regulated | 32 |
| ENSG00000081479 | LRP2 | 4036 | Up-regulated | 33 |
| ENSG00000166828 | SCNN1G | 6340 | Up-regulated | 34 |
| ENSG00000184719 | RNLS | 55328 | Up-regulated | 35 |
| ENSG00000136155 | SCEL | 8796 | Up-regulated | 36 |
| ENSG00000253898 | LINC01419 | NA | Up-regulated | 37 |
| ENSG00000183742 | MACC1 | 346389 | Up-regulated | 38 |
| ENSG00000154654 | NCAM2 | 4685 | Up-regulated | 39 |
| ENSG00000140873 | ADAMTS18 | 170692 | Up-regulated | 40 |
| ENSG00000148346 | LCN2 | 3934 | Up-regulated | 41 |
| ENSG00000233834 | AC005083.1 | NA | Up-regulated | 42 |
| ENSG00000237438 | CECR7 | NA | Up-regulated | 43 |
| ENSG00000124939 | SCGB2A1 | 4246 | Up-regulated | 44 |
| ENSG00000115221 | ITGB6 | 3694 | Up-regulated | 45 |
| ENSG00000273203 | AC006946.2 | NA | Up-regulated | 46 |
| ENSG00000253313 | C1orf210 | 149466 | Up-regulated | 47 |
| ENSG00000243236 | GSTA9P | NA | Up-regulated | 48 |
| ENSG00000071909 | MYO3B | 140469 | Up-regulated | 49 |
| ENSG00000188511 | C22orf34 | 348645 | Up-regulated | 50 |
| ENSG00000267795 | SMIM22 | 440335 | Up-regulated | 51 |
| ENSG00000250606 | NA | NA | Up-regulated | 52 |
| ENSG00000130701 | RBBP8NL | 140893 | Up-regulated | 53 |
| ENSG00000137648 | TMPRSS4 | 56649 | Up-regulated | 54 |
| ENSG00000196189 | SEMA4A | 64218 | Up-regulated | 55 |
| ENSG00000178750 | STX19 | 415117 | Up-regulated | 56 |
| ENSG00000070190 | DAPP1 | 27071 | Up-regulated | 57 |
| ENSG00000230099 | TRBV5-4 | NA | Up-regulated | 58 |
| ENSG00000128422 | KRT17 | 3872 | Up-regulated | 59 |
| ENSG00000158639 | PAGE5 | 90737 | Up-regulated | 60 |
| ENSG00000152822 | GRM1 | 2911 | Up-regulated | 61 |
| ENSG00000152779 | SLC16A12 | 387700 | Up-regulated | 62 |
| ENSG00000189143 | CLDN4 | 1364 | Up-regulated | 63 |
| ENSG00000256001 | AC079949.1 | NA | Up-regulated | 64 |
| ENSG00000144648 | ACKR2 | 1238 | Up-regulated | 65 |
| ENSG00000140297 | GCNT3 | 9245 | Up-regulated | 66 |
| ENSG00000064270 | ATP2C2 | 9914 | Up-regulated | 67 |
| ENSG00000185156 | MFSD6L | 162387 | Up-regulated | 68 |
| ENSG00000143217 | NECTIN4 | 81607 | Up-regulated | 69 |
| ENSG00000117228 | GBP1 | 2633 | Up-regulated | 70 |
| ENSG00000110195 | FOLR1 | 2348 | Up-regulated | 71 |
| ENSG00000257084 | MIR200CHG | NA | Up-regulated | 72 |
| ENSG00000052344 | PRSS8 | 5652 | Up-regulated | 73 |
| ENSG00000253417 | LINC02159 | NA | Up-regulated | 74 |
| ENSG00000188488 | SERPINA5 | 5104 | Up-regulated | 75 |
| ENSG00000273328 | AC099329.2 | NA | Up-regulated | 76 |
| ENSG00000104490 | NCALD | 83988 | Up-regulated | 77 |
| ENSG00000205642 | VCX3B | 425054 | Up-regulated | 78 |
| ENSG00000066230 | SLC9A3 | 6550 | Up-regulated | 79 |
| ENSG00000248713 | C4orf54 | 285556 | Up-regulated | 80 |
| ENSG00000165023 | DIRAS2 | 54769 | Up-regulated | 81 |
| ENSG00000111846 | GCNT2 | 2651 | Up-regulated | 82 |
| ENSG00000105523 | FAM83E | 54854 | Up-regulated | 83 |
| ENSG00000189299 | FOXR2 | 139628 | Up-regulated | 84 |
| ENSG00000139946 | PELI2 | 57161 | Up-regulated | 85 |
| ENSG00000180432 | CYP8B1 | 1582 | Up-regulated | 86 |
| ENSG00000205336 | ADGRG1 | 9289 | Up-regulated | 87 |
| ENSG00000101276 | SLC52A3 | 113278 | Up-regulated | 88 |
| ENSG00000155066 | PROM2 | 150696 | Up-regulated | 89 |
| ENSG00000243709 | LEFTY1 | 10637 | Up-regulated | 90 |
| ENSG00000078114 | NEBL | 10529 | Up-regulated | 91 |
| ENSG00000146374 | RSPO3 | 84870 | Up-regulated | 92 |
| ENSG00000196557 | CACNA1H | 8912 | Up-regulated | 93 |
| ENSG00000179178 | TMEM125 | 128218 | Up-regulated | 94 |
| ENSG00000204682 | CASC10 | 399726 | Up-regulated | 95 |
| ENSG00000189108 | IL1RAPL2 | 26280 | Up-regulated | 96 |
| ENSG00000183378 | OVCH2 | 341277 | Up-regulated | 97 |
| ENSG00000166558 | SLC38A8 | 146167 | Up-regulated | 98 |
| ENSG00000115339 | GALNT3 | 2591 | Up-regulated | 99 |
| ENSG00000133962 | CATSPERB | 79820 | Up-regulated | 100 |
| ENSG00000158578 | ALAS2 | 212 | Up-regulated | 101 |
| ENSG00000146411 | SLC2A12 | 154091 | Up-regulated | 102 |
| ENSG00000162069 | BICDL2 | 146439 | Up-regulated | 103 |
| ENSG00000168916 | ZNF608 | 57507 | Up-regulated | 104 |
| ENSG00000047457 | CP | 1356 | Up-regulated | 105 |
| ENSG00000250366 | TUNAR | NA | Up-regulated | 106 |
| ENSG00000129151 | BBOX1 | 8424 | Up-regulated | 107 |
| ENSG00000205890 | AC108134.1 | NA | Up-regulated | 108 |
| ENSG00000272141 | AL390719.2 | NA | Up-regulated | 109 |
| ENSG00000233198 | RNF224 | 643596 | Up-regulated | 110 |
| ENSG00000136267 | DGKB | 1607 | Up-regulated | 111 |
| ENSG00000272189 | AL024508.2 | NA | Up-regulated | 112 |
| ENSG00000188897 | AC099489.1 | 400499 | Up-regulated | 113 |
| ENSG00000079215 | SLC1A3 | 6507 | Up-regulated | 114 |
| ENSG00000176945 | MUC20 | 200958 | Up-regulated | 115 |
| ENSG00000115705 | TPO | 7173 | Up-regulated | 116 |
| ENSG00000170421 | KRT8 | 3856 | Up-regulated | 117 |
| ENSG00000258791 | LINC00520 | NA | Up-regulated | 118 |
| ENSG00000140505 | CYP1A2 | 1544 | Up-regulated | 119 |
| ENSG00000197249 | SERPINA1 | 5265 | Up-regulated | 120 |
| ENSG00000204136 | GGTA1P | 2681 | Up-regulated | 121 |
| ENSG00000181885 | CLDN7 | 1366 | Up-regulated | 122 |
| ENSG00000260581 | AC011374.1 | NA | Up-regulated | 123 |
| ENSG00000173175 | ADCY5 | 111 | Up-regulated | 124 |
| ENSG00000224520 | KRT8P45 | NA | Up-regulated | 125 |
| ENSG00000107796 | ACTA2 | 59 | Up-regulated | 126 |
| ENSG00000004468 | CD38 | 952 | Up-regulated | 127 |
| ENSG00000242640 | RPS29P11 | NA | Up-regulated | 128 |
| ENSG00000271826 | PLS3-AS1 | NA | Up-regulated | 129 |
| ENSG00000174502 | SLC26A9 | 115019 | Up-regulated | 130 |
| ENSG00000203635 | AC 144450.1 | NA | Up-regulated | 131 |
| ENSG00000272703 | AP005137.2 | NA | Up-regulated | 132 |
| ENSG00000149573 | MPZL2 | 10205 | Up-regulated | 133 |
| ENSG00000231672 | DIRC3 | NA | Up-regulated | 134 |
| ENSG00000102678 | FGF9 | 2254 | Up-regulated | 135 |
| ENSG00000261804 | AC007342.4 | NA | Up-regulated | 136 |
| ENSG00000062038 | CDH3 | 1001 | Up-regulated | 137 |
| ENSG00000135373 | EHF | 26298 | Up-regulated | 138 |
| ENSG00000163817 | SLC6A20 | 54716 | Up-regulated | 139 |
| ENSG00000130508 | PXDN | 7837 | Up-regulated | 140 |
| ENSG00000131037 | EPS8L1 | 54869 | Up-regulated | 141 |
| ENSG00000261122 | LINC02167 | NA | Up-regulated | 142 |
| ENSG00000196188 | CTSE | 1510 | Up-regulated | 143 |
| ENSG00000250420 | AACSP1 | NA | Up-regulated | 144 |
| ENSG00000163132 | MSX1 | 4487 | Up-regulated | 145 |
| ENSG00000234147 | AL035446.1 | NA | Up-regulated | 146 |
| ENSG00000204661 | C5orf60 | 285679 | Up-regulated | 147 |
| ENSG00000261068 | AL512274.1 | NA | Up-regulated | 148 |
| ENSG00000170454 | KRT75 | 9119 | Up-regulated | 149 |
| ENSG00000215386 | MIR99AHG | NA | Up-regulated | 150 |
| ENSG00000115590 | IL1R2 | 7850 | Up-regulated | 151 |
| ENSG00000262714 | AC007342.5 | NA | Up-regulated | 152 |
| ENSG00000120549 | KIAA1217 | 56243 | Up-regulated | 153 |
| ENSG00000149972 | CNTN5 | 53942 | Up-regulated | 154 |
| ENSG00000254429 | AP001972.1 | NA | Up-regulated | 155 |
| ENSG00000165025 | SYK | 6850 | Up-regulated | 156 |
| ENSG00000124429 | POF1B | 79983 | Up-regulated | 157 |
| ENSG00000139679 | LPAR6 | 10161 | Up-regulated | 158 |
| ENSG00000143603 | KCNN3 | 3782 | Up-regulated | 159 |
| ENSG00000187017 | ESPN | 83715 | Up-regulated | 160 |
| ENSG00000135114 | OASL | 8638 | Up-regulated | 161 |
| ENSG00000228933 | AC107419.1 | NA | Up-regulated | 162 |
| ENSG00000260711 | AL121839.2 | NA | Up-regulated | 163 |
| ENSG00000184368 | MAP7D2 | 256714 | Up-regulated | 164 |
| ENSG00000154556 | SORBS2 | 8470 | Up-regulated | 165 |
| ENSG00000119922 | IFIT2 | 3433 | Up-regulated | 166 |
| ENSG00000164197 | RNF180 | 285671 | Up-regulated | 167 |
| ENSG00000070731 | ST6GALNAC2 | 10610 | Up-regulated | 168 |
| ENSG00000269067 | ZNF728 | 388523 | Up-regulated | 169 |
| ENSG00000157765 | SLC34A2 | 10568 | Up-regulated | 170 |
| ENSG00000184792 | OSBP2 | 23762 | Up-regulated | 171 |
| ENSG00000244586 | WNT5A-AS1 | NA | Up-regulated | 172 |
| ENSG00000183117 | CSMD1 | 64478 | Up-regulated | 173 |
| ENSG00000272081 | AC008972.2 | NA | Up-regulated | 174 |
| ENSG00000039068 | CDH1 | 999 | Up-regulated | 175 |
| ENSG00000113924 | HGD | 3081 | Up-regulated | 176 |
| ENSG00000118785 | SPP1 | 6696 | Up-regulated | 177 |
| ENSG00000120162 | MOB3B | 79817 | Up-regulated | 178 |
| ENSG00000196878 | LAMB3 | 3914 | Up-regulated | 179 |
| ENSG00000120278 | PLEKHG1 | 57480 | Up-regulated | 180 |
| ENSG00000230006 | ANKRD36BP2 | NA | Up-regulated | 181 |
| ENSG00000114251 | WNT5A | 7474 | Up-regulated | 182 |
| ENSG00000240668 | KRT8P36 | NA | Up-regulated | 183 |
| ENSG00000196139 | AKR1C3 | 8644 | Up-regulated | 184 |
| ENSG00000151322 | NPAS3 | 64067 | Up-regulated | 185 |
| ENSG00000139714 | MORN3 | 283385 | Up-regulated | 186 |
| ENSG00000254285 | KRT8P3 | NA | Up-regulated | 187 |
| ENSG00000143365 | RORC | 6097 | Up-regulated | 188 |
| ENSG00000160588 | MPZL3 | 196264 | Up-regulated | 189 |
| ENSG00000175318 | GRAMD2A | 196996 | Up-regulated | 190 |
| ENSG00000151632 | AKR1C2 | 1646 | Up-regulated | 191 |
| ENSG00000118407 | FILIP1 | 27145 | Up-regulated | 192 |
| ENSG00000146904 | EPHA1 | 2041 | Up-regulated | 193 |
| ENSG00000066629 | EML1 | 2009 | Up-regulated | 194 |
| ENSG00000122012 | SV2C | 22987 | Up-regulated | 195 |
| ENSG00000180758 | GPR157 | 80045 | Up-regulated | 196 |
| ENSG00000196482 | ESRRG | 2104 | Up-regulated | 197 |
| ENSG00000178078 | STAP2 | 55620 | Up-regulated | 198 |
| ENSG00000135205 | CCDC146 | 57639 | Up-regulated | 199 |
| ENSG00000137486 | ARRB1 | 408 | Up-regulated | 200 |
| ENSG00000271926 | AC008972.1 | NA | Up-regulated | 201 |
| ENSG00000103449 | SALL1 | 6299 | Up-regulated | 202 |
| ENSG00000165168 | CYBB | 1536 | Up-regulated | 203 |
| ENSG00000131242 | RAB11FIP4 | 84440 | Up-regulated | 204 |
| ENSG00000138670 | RASGEF1B | 153020 | Up-regulated | 205 |
| ENSG00000183785 | TUBA8 | 51807 | Up-regulated | 206 |
| ENSG00000041982 | TNC | 3371 | Up-regulated | 207 |
| ENSG00000164120 | HPGD | 3248 | Up-regulated | 208 |
| ENSG00000173698 | ADGRG2 | 10149 | Up-regulated | 209 |
| ENSG00000150551 | LYPD1 | 116372 | Up-regulated | 210 |
| ENSG00000184226 | PCDH9 | 5101 | Up-regulated | 211 |
| ENSG00000110693 | SOX6 | 55553 | Up-regulated | 212 |
| ENSG00000168140 | VASN | 114990 | Up-regulated | 213 |
| ENSG00000197165 | SULT1A2 | 6799 | Up-regulated | 214 |
| ENSG00000272068 | AL365181.2 | NA | Up-regulated | 215 |
| ENSG00000005102 | MEOX1 | 4222 | Up-regulated | 216 |
| ENSG00000198774 | RASSF9 | 9182 | Up-regulated | 217 |
| ENSG00000073282 | TP63 | 8626 | Up-regulated | 218 |
| ENSG00000171243 | SOSTDC1 | 25928 | Up-regulated | 219 |
| ENSG00000138161 | CUZD1 | 50624 | Up-regulated | 220 |
| ENSG00000081818 | PCDHB4 | 56131 | Up-regulated | 221 |
| ENSG00000176046 | NUPR1 | 26471 | Up-regulated | 222 |
| ENSG00000151320 | AKAP6 | 9472 | Up-regulated | 223 |
| ENSG00000157992 | KRTCAP3 | 200634 | Up-regulated | 224 |
| ENSG00000168952 | STXBP6 | 29091 | Up-regulated | 225 |
| ENSG00000156463 | SH3RF2 | 153769 | Up-regulated | 226 |
| ENSG00000115290 | GRB14 | 2888 | Up-regulated | 227 |
| ENSG00000054179 | ENTPD2 | 954 | Up-regulated | 228 |
| ENSG00000119411 | BSPRY | 54836 | Up-regulated | 229 |
| ENSG00000136167 | LCP1 | 3936 | Up-regulated | 230 |
| ENSG00000167608 | TMC4 | 147798 | Up-regulated | 231 |
| ENSG00000132874 | SLC14A2 | 8170 | Up-regulated | 232 |
| ENSG00000078018 | MAP2 | 4133 | Up-regulated | 233 |
| ENSG00000114854 | TNNC1 | 7134 | Up-regulated | 234 |
| ENSG00000105519 | CAPS | 828 | Up-regulated | 235 |
| ENSG00000076864 | RAP1GAP | 5909 | Up-regulated | 236 |
| ENSG00000078401 | EDN1 | 1906 | Up-regulated | 237 |
| ENSG00000165929 | TC2N | 123036 | Up-regulated | 238 |
| ENSG00000149418 | ST14 | 6768 | Up-regulated | 239 |
| ENSG00000175707 | KDF1 | 126695 | Up-regulated | 240 |
| ENSG00000249751 | ECSCR | 641700 | Up-regulated | 241 |
| ENSG00000172201 | ID4 | 3400 | Up-regulated | 242 |
| ENSG00000137558 | PI15 | 51050 | Up-regulated | 243 |
| ENSG00000050628 | PTGER3 | 5733 | Up-regulated | 244 |
| ENSG00000145743 | FBXL17 | 64839 | Up-regulated | 245 |
| ENSG00000188112 | C6orfl32 | 647024 | Up-regulated | 246 |
| ENSG00000203727 | SAMD5 | 389432 | Up-regulated | 247 |
| ENSG00000130707 | ASS1 | 445 | Up-regulated | 248 |
| ENSG00000091592 | NLRP1 | 728392 | Up-regulated | 249 |
| ENSG00000091592 | NLRP1 | 22861 | Up-regulated | 250 |
| ENSG00000006047 | YBX2 | 51087 | Up-regulated | 251 |
| ENSG00000102890 | ELMO3 | 79767 | Up-regulated | 252 |
| ENSG00000105855 | ITGB8 | 3696 | Up-regulated | 253 |
| ENSG00000138821 | SLC39A8 | 64116 | Up-regulated | 254 |
| ENSG00000220023 | NA | NA | Up-regulated | 255 |
| ENSG00000198626 | RYR2 | 6262 | Up-regulated | 256 |
| ENSG00000143816 | WNT9A | 7483 | Up-regulated | 257 |
| ENSG00000178538 | CA8 | 767 | Up-regulated | 258 |
| ENSG00000227184 | NA | NA | Up-regulated | 259 |
| ENSG00000164761 | TNFRSF11B | 4982 | Up-regulated | 260 |
| ENSG00000203499 | IQANK1 | NA | Up-regulated | 261 |
| ENSG00000140092 | FBLN5 | 10516 | Up-regulated | 262 |
| ENSG00000130545 | CRB3 | 92359 | Up-regulated | 263 |
| ENSG00000117595 | IRF6 | 3664 | Up-regulated | 264 |
| ENSG00000132205 | EMILIN2 | 84034 | Up-regulated | 265 |
| ENSG00000136842 | TMOD1 | 7111 | Up-regulated | 266 |
| ENSG00000134532 | SOX5 | 6660 | Up-regulated | 267 |
| ENSG00000205213 | LGR4 | 55366 | Up-regulated | 268 |
| ENSG00000176788 | BASP1 | 10409 | Up-regulated | 269 |
| ENSG00000162772 | ATF3 | 467 | Up-regulated | 270 |
| ENSG00000010810 | FYN | 2534 | Up-regulated | 271 |
| ENSG00000035115 | SH3YL1 | 26751 | Up-regulated | 272 |
| ENSG00000184349 | EFNA5 | 1946 | Up-regulated | 273 |
| ENSG00000119888 | EPCAM | 4072 | Up-regulated | 274 |
| ENSG00000165474 | GJB2 | 2706 | Up-regulated | 275 |
| ENSG00000129354 | AP1M2 | 10053 | Up-regulated | 276 |
| ENSG00000144278 | GALNT13 | 114805 | Up-regulated | 277 |
| ENSG00000159166 | LAD1 | 3898 | Up-regulated | 278 |
| ENSG00000047597 | XK | 7504 | Up-regulated | 279 |
| ENSG00000130396 | AFDN | 4301 | Up-regulated | 280 |
| ENSG00000151726 | ACSL1 | 2180 | Up-regulated | 281 |
| ENSG00000102038 | SMARCA1 | 6594 | Down-regulated | 1 |
| ENSG00000005249 | PRKAR2B | 5577 | Down-regulated | 2 |
| ENSG00000106789 | CORO2A | 7464 | Down-regulated | 3 |
| ENSG00000197956 | S100A6 | 6277 | Down-regulated | 4 |
| ENSG00000135842 | FAM129A | 116496 | Down-regulated | 5 |
| ENSG00000127990 | SGCE | 8910 | Down-regulated | 6 |
| ENSG00000141756 | FKBP10 | 60681 | Down-regulated | 7 |
| ENSG00000175505 | CLCF1 | 23529 | Down-regulated | 8 |
| ENSG00000198930 | CSAG1 | 158511 | Down-regulated | 9 |
| ENSG00000089597 | GANAB | 23193 | Down-regulated | 10 |
| ENSG00000204525 | HLA-C | 3107 | Down-regulated | 11 |
| ENSG00000006534 | ALDH3B1 | 221 | Down-regulated | 12 |
| ENSG00000067057 | PFKP | 5214 | Down-regulated | 13 |
| ENSG00000196754 | S100A2 | 6273 | Down-regulated | 14 |
| ENSG00000206052 | DOK6 | 220164 | Down-regulated | 15 |
| ENSG00000213694 | S1PR3 | 1903 | Down-regulated | 16 |
| ENSG00000137936 | BCAR3 | 8412 | Down-regulated | 17 |
| ENSG00000198682 | PAPSS2 | 9060 | Down-regulated | 18 |
| ENSG00000196154 | S100A4 | 6275 | Down-regulated | 19 |
| ENSG00000123146 | ADGRE5 | 976 | Down-regulated | 20 |
| ENSG00000198624 | CCDC69 | 26112 | Down-regulated | 21 |
| ENSG00000131389 | SLC6A6 | 6533 | Down-regulated | 22 |
| ENSG00000111674 | ENO2 | 2026 | Down-regulated | 23 |
| ENSG00000213401 | MAGEA12 | 4111 | Down-regulated | 24 |
| ENSG00000074211 | PPP2R2C | 5522 | Down-regulated | 25 |
| ENSG00000170500 | LONRF2 | 164832 | Down-regulated | 26 |
| ENSG00000172638 | EFEMP2 | 30008 | Down-regulated | 27 |
| ENSG00000187720 | THSD4 | 79875 | Down-regulated | 28 |
| ENSG00000196155 | PLEKHG4 | 25894 | Down-regulated | 29 |
| ENSG00000175556 | LONRF3 | 79836 | Down-regulated | 30 |
| ENSG00000072657 | TRHDE | 29953 | Down-regulated | 31 |
| ENSG00000159164 | SV2A | 9900 | Down-regulated | 32 |
| ENSG00000144824 | PHLDB2 | 90102 | Down-regulated | 33 |
| ENSG00000165806 | CASP7 | 840 | Down-regulated | 34 |
| ENSG00000176490 | DIRAS1 | 148252 | Down-regulated | 35 |
| ENSG00000135905 | DOCK10 | 55619 | Down-regulated | 36 |
| ENSG00000105048 | TNNT1 | 7138 | Down-regulated | 37 |
| ENSG00000158164 | TMSB15A | 286527 | Down-regulated | 38 |
| ENSG00000158164 | TMSB15A | 11013 | Down-regulated | 39 |
| ENSG00000253910 | PCDHGB2 | 56103 | Down-regulated | 40 |
| ENSG00000086289 | EPDR1 | 54749 | Down-regulated | 41 |
| ENSG00000105137 | SYDE1 | 85360 | Down-regulated | 42 |
| ENSG00000100979 | PLTP | 5360 | Down-regulated | 43 |
| ENSG00000205978 | NYNRIN | 57523 | Down-regulated | 44 |
| ENSG00000168077 | SCARA3 | 51435 | Down-regulated | 45 |
| ENSG00000185904 | LINC00839 | NA | Down-regulated | 46 |
| ENSG00000100167 | Sep-03 | 55964 | Down-regulated | 47 |
| ENSG00000126561 | STAT5A | 6776 | Down-regulated | 48 |
| ENSG00000104870 | FCGRT | 2217 | Down-regulated | 49 |
| ENSG00000175928 | LRRN1 | 57633 | Down-regulated | 50 |
| ENSG00000197043 | ANXA6 | 309 | Down-regulated | 51 |
| ENSG00000103710 | RASL12 | 51285 | Down-regulated | 52 |
| ENSG00000108797 | CNTNAP1 | 8506 | Down-regulated | 53 |
| ENSG00000166450 | PRTG | 283659 | Down-regulated | 54 |
| ENSG00000075618 | FSCN1 | 6624 | Down-regulated | 55 |
| ENSG00000100228 | RAB36 | 9609 | Down-regulated | 56 |
| ENSG00000184867 | ARMCX2 | 9823 | Down-regulated | 57 |
| ENSG00000159263 | SIM2 | 6493 | Down-regulated | 58 |
| ENSG00000130005 | GAMT | 2593 | Down-regulated | 59 |
| ENSG00000129675 | ARHGEF6 | 9459 | Down-regulated | 60 |
| ENSG00000066248 | NGEF | 25791 | Down-regulated | 61 |
| ENSG00000108387 | Sep-04 | 5414 | Down-regulated | 62 |
| ENSG00000198832 | SELENOM | 140606 | Down-regulated | 63 |
| ENSG00000151617 | EDNRA | 1909 | Down-regulated | 64 |
| ENSG00000184258 | CDR1 | 1038 | Down-regulated | 65 |
| ENSG00000135424 | ITGA7 | 3679 | Down-regulated | 66 |
| ENSG00000005961 | ITGA2B | 3674 | Down-regulated | 67 |
| ENSG00000184838 | PRR16 | 51334 | Down-regulated | 68 |
| ENSG00000163909 | HEYL | 26508 | Down-regulated | 69 |
| ENSG00000182013 | PNMA8A | 55228 | Down-regulated | 70 |
| ENSG00000169126 | ARMC4 | 55130 | Down-regulated | 71 |
| ENSG00000106366 | SERPINE1 | 5054 | Down-regulated | 72 |
| ENSG00000101955 | SRPX | 8406 | Down-regulated | 73 |
| ENSG00000136274 | NACAD | 23148 | Down-regulated | 74 |
| ENSG00000151572 | ANO4 | 121601 | Down-regulated | 75 |
| ENSG00000163053 | SLC16A14 | 151473 | Down-regulated | 76 |
| ENSG00000124507 | PACSIN1 | 29993 | Down-regulated | 77 |
| ENSG00000106665 | CLIP2 | 7461 | Down-regulated | 78 |
| ENSG00000117289 | NA | NA | Down-regulated | 79 |
| ENSG00000116962 | NID1 | 4811 | Down-regulated | 80 |
| ENSG00000156299 | TIAM1 | 7074 | Down-regulated | 81 |
| ENSG00000112183 | RBM24 | 221662 | Down-regulated | 82 |
| ENSG00000182272 | B4GALNT4 | 338707 | Down-regulated | 83 |
| ENSG00000136653 | NA | NA | Down-regulated | 84 |
| ENSG00000116729 | WLS | 79971 | Down-regulated | 85 |
| ENSG00000177508 | IRX3 | 79191 | Down-regulated | 86 |
| ENSG00000159403 | C1R | 715 | Down-regulated | 87 |
| ENSG00000129244 | ATP1B2 | 482 | Down-regulated | 88 |
| ENSG00000005884 | ITGA3 | 3675 | Down-regulated | 89 |
| ENSG00000092964 | DPYSL2 | 1808 | Down-regulated | 90 |
| ENSG00000148143 | ZNF462 | 58499 | Down-regulated | 91 |
| ENSG00000136490 | LIMD2 | 80774 | Down-regulated | 92 |
| ENSG00000020181 | ADGRA2 | 25960 | Down-regulated | 93 |
| ENSG00000101938 | CHRDL1 | 91851 | Down-regulated | 94 |
| ENSG00000143369 | ECM1 | 1893 | Down-regulated | 95 |
| ENSG00000057019 | DCBLD2 | 131566 | Down-regulated | 96 |
| ENSG00000254122 | PCDHGB7 | 56099 | Down-regulated | 97 |
| ENSG00000124813 | RUNX2 | 860 | Down-regulated | 98 |
| ENSG00000122574 | WIPF3 | 644150 | Down-regulated | 99 |
| ENSG00000151474 | FRMD4A | 55691 | Down-regulated | 100 |
| ENSG00000127124 | HIVEP3 | 59269 | Down-regulated | 101 |
| ENSG00000135929 | CYP27A1 | 1593 | Down-regulated | 102 |
| ENSG00000146013 | GFRA3 | 2676 | Down-regulated | 103 |
| ENSG00000147872 | PLIN2 | 123 | Down-regulated | 104 |
| ENSG00000136542 | GALNT5 | 11227 | Down-regulated | 105 |
| ENSG00000091844 | RGS17 | 26575 | Down-regulated | 106 |
| ENSG00000007314 | SCN4A | 6329 | Down-regulated | 107 |
| ENSG00000006062 | MAP3K14 | 9020 | Down-regulated | 108 |
| ENSG00000197291 | RAMP2-AS1 | NA | Down-regulated | 109 |
| ENSG00000182963 | GJC1 | 10052 | Down-regulated | 110 |
| ENSG00000134548 | SPX | 80763 | Down-regulated | 111 |
| ENSG00000183087 | GAS6 | 2621 | Down-regulated | 112 |
| ENSG00000050165 | DKK3 | 27122 | Down-regulated | 113 |
| ENSG00000031081 | ARHGAP31 | 57514 | Down-regulated | 114 |
| ENSG00000101187 | SLCO4A1 | 28231 | Down-regulated | 115 |
| ENSG00000149260 | CAPN5 | 726 | Down-regulated | 116 |
| ENSG00000111817 | DSE | 29940 | Down-regulated | 117 |
| ENSG00000100097 | LGALS1 | 3956 | Down-regulated | 118 |
| ENSG00000107957 | SH3PXD2A | 9644 | Down-regulated | 119 |
| ENSG00000169862 | CTNND2 | 1501 | Down-regulated | 120 |
| ENSG00000128656 | CHN1 | 1123 | Down-regulated | 121 |
| ENSG00000020633 | RUNX3 | 864 | Down-regulated | 122 |
| ENSG00000196876 | SCN8A | 6334 | Down-regulated | 123 |
| ENSG00000154310 | TNIK | 23043 | Down-regulated | 124 |
| ENSG00000043143 | JADE2 | 23338 | Down-regulated | 125 |
| ENSG00000144712 | CAND2 | 23066 | Down-regulated | 126 |
| ENSG00000174004 | NRROS | 375387 | Down-regulated | 127 |
| ENSG00000131477 | RAMP2 | 10266 | Down-regulated | 128 |
| ENSG00000035862 | TIMP2 | 7077 | Down-regulated | 129 |
| ENSG00000130203 | APOE | 348 | Down-regulated | 130 |
| ENSG00000167880 | EVPL | 2125 | Down-regulated | 131 |
| ENSG00000138311 | ZNF365 | 22891 | Down-regulated | 132 |
| ENSG00000149596 | JPH2 | 57158 | Down-regulated | 133 |
| ENSG00000170745 | KCNS3 | 3790 | Down-regulated | 134 |
| ENSG00000128849 | CGNL1 | 84952 | Down-regulated | 135 |
| ENSG00000117600 | PLPPR4 | 9890 | Down-regulated | 136 |
| ENSG00000137285 | TUBB2B | 347733 | Down-regulated | 137 |
| ENSG00000150051 | MKX | 283078 | Down-regulated | 138 |
| ENSG00000128335 | APOL2 | 23780 | Down-regulated | 139 |
| ENSG00000144642 | RBMS3 | 27303 | Down-regulated | 140 |
| ENSG00000267750 | RUNDC3A-AS1 | NA | Down-regulated | 141 |
| ENSG00000110811 | P3H3 | 10536 | Down-regulated | 142 |
| ENSG00000170537 | TMC7 | 79905 | Down-regulated | 143 |
| ENSG00000139629 | GALNT6 | 11226 | Down-regulated | 144 |
| ENSG00000087303 | NID2 | 22795 | Down-regulated | 145 |
| ENSG00000065534 | MYLK | 4638 | Down-regulated | 146 |
| ENSG00000170743 | SYT9 | 143425 | Down-regulated | 147 |
| ENSG00000146966 | DENND2A | 27147 | Down-regulated | 148 |
| ENSG00000074370 | ATP2A3 | 489 | Down-regulated | 149 |
| ENSG00000115641 | FHL2 | 2274 | Down-regulated | 150 |
| ENSG00000105974 | CAV1 | 857 | Down-regulated | 151 |
| ENSG00000178860 | MSC | 9242 | Down-regulated | 152 |
| ENSG00000131459 | GFPT2 | 9945 | Down-regulated | 153 |
| ENSG00000114948 | ADAM23 | 8745 | Down-regulated | 154 |
| ENSG00000066032 | CTNNA2 | 1496 | Down-regulated | 155 |
| ENSG00000183578 | TNFAIP8L3 | 388121 | Down-regulated | 156 |
| ENSG00000178568 | ERBB4 | 2066 | Down-regulated | 157 |
| ENSG00000197977 | ELOVL2 | 54898 | Down-regulated | 158 |
| ENSG00000104998 | IL27RA | 9466 | Down-regulated | 159 |
| ENSG00000204128 | C2orf72 | 257407 | Down-regulated | 160 |
| ENSG00000007062 | PROM1 | 8842 | Down-regulated | 161 |
| ENSG00000171408 | PDE7B | 27115 | Down-regulated | 162 |
| ENSG00000113790 | EHHADH | 1962 | Down-regulated | 163 |
| ENSG00000250305 | TRMT9B | 57604 | Down-regulated | 164 |
| ENSG00000162989 | KCNJ3 | 3760 | Down-regulated | 165 |
| ENSG00000100599 | RIN3 | 79890 | Down-regulated | 166 |
| ENSG00000135919 | SERPINE2 | 5270 | Down-regulated | 167 |
| ENSG00000105329 | TGFB1 | 7040 | Down-regulated | 168 |
| ENSG00000183145 | RIPPLY3 | 53820 | Down-regulated | 169 |
| ENSG00000113448 | PDE4D | 5144 | Down-regulated | 170 |
| ENSG00000092969 | TGFB2 | 7042 | Down-regulated | 171 |
| ENSG00000185652 | NTF3 | 4908 | Down-regulated | 172 |
| ENSG00000231789 | PIK3CD-AS2 | NA | Down-regulated | 173 |
| ENSG00000111052 | LIN7A | 8825 | Down-regulated | 174 |
| ENSG00000109452 | INPP4B | 8821 | Down-regulated | 175 |
| ENSG00000184194 | GPR173 | 54328 | Down-regulated | 176 |
| ENSG00000109099 | PMP22 | 5376 | Down-regulated | 177 |
| ENSG00000066468 | FGFR2 | 2263 | Down-regulated | 178 |
| ENSG00000186684 | CYP27C1 | 339761 | Down-regulated | 179 |
| ENSG00000168748 | CA7 | 766 | Down-regulated | 180 |
| ENSG00000175175 | PPM1E | 22843 | Down-regulated | 181 |
| ENSG00000152495 | CAMK4 | 814 | Down-regulated | 182 |
| ENSG00000180801 | ARSJ | 79642 | Down-regulated | 183 |
| ENSG00000128872 | TMOD2 | 29767 | Down-regulated | 184 |
| ENSG00000159784 | FAM131B | 9715 | Down-regulated | 185 |
| ENSG00000122861 | PLAU | 5328 | Down-regulated | 186 |
| ENSG00000177469 | CAVIN1 | 284119 | Down-regulated | 187 |
| ENSG00000198885 | ITPRIPL1 | 150771 | Down-regulated | 188 |
| ENSG00000157227 | MMP14 | 4323 | Down-regulated | 189 |
| ENSG00000250386 | AC233724.10 | NA | Down-regulated | 190 |
| ENSG00000066735 | KIF26A | 26153 | Down-regulated | 191 |
| ENSG00000113327 | GABRG2 | 2566 | Down-regulated | 192 |
| ENSG00000013016 | EHD3 | 30845 | Down-regulated | 193 |
| ENSG00000157064 | NMNAT2 | 23057 | Down-regulated | 194 |
| ENSG00000259498 | TPM1-AS | NA | Down-regulated | 195 |
| ENSG00000184922 | FMNL1 | 752 | Down-regulated | 196 |
| ENSG00000168243 | GNG4 | 2786 | Down-regulated | 197 |
| ENSG00000180611 | MB21D2 | 151963 | Down-regulated | 198 |
| ENSG00000175592 | FOSL1 | 8061 | Down-regulated | 199 |
| ENSG00000126970 | ZC4H2 | 55906 | Down-regulated | 200 |
| ENSG00000153246 | PLA2R1 | 22925 | Down-regulated | 201 |
| ENSG00000088992 | TESC | 54997 | Down-regulated | 202 |
| ENSG00000156103 | MMP16 | 4325 | Down-regulated | 203 |
| ENSG00000166780 | C16orf45 | 89927 | Down-regulated | 204 |
| ENSG00000166750 | SLFN5 | 162394 | Down-regulated | 205 |
| ENSG00000143195 | ILDR2 | 387597 | Down-regulated | 206 |
| ENSG00000242779 | ZNF702P | NA | Down-regulated | 207 |
| ENSG00000112320 | SOBP | 55084 | Down-regulated | 208 |
| ENSG00000121361 | KCNJ8 | 3764 | Down-regulated | 209 |
| ENSG00000107147 | KCNT1 | 57582 | Down-regulated | 210 |
| ENSG00000118596 | SLC16A7 | 9194 | Down-regulated | 211 |
| ENSG00000118257 | NRP2 | 8828 | Down-regulated | 212 |
| ENSG00000131018 | SYNE1 | 23345 | Down-regulated | 213 |
| ENSG00000067715 | SYT1 | 6857 | Down-regulated | 214 |
| ENSG00000133169 | BEX1 | 55859 | Down-regulated | 215 |
| ENSG00000171812 | COL8A2 | 1296 | Down-regulated | 216 |
| ENSG00000101000 | PROCR | 10544 | Down-regulated | 217 |
| ENSG00000168743 | NPNT | 255743 | Down-regulated | 218 |
| ENSG00000146216 | TTBK1 | 84630 | Down-regulated | 219 |
| ENSG00000143355 | LHX9 | 56956 | Down-regulated | 220 |
| ENSG00000159231 | CBR3 | 874 | Down-regulated | 221 |
| ENSG00000140403 | DNAJA4 | 55466 | Down-regulated | 222 |
| ENSG00000141750 | STAC2 | 342667 | Down-regulated | 223 |
| ENSG00000013588 | GPRC5A | 9052 | Down-regulated | 224 |
| ENSG00000153993 | SEMA3D | 223117 | Down-regulated | 225 |
| ENSG00000168685 | IL7R | 3575 | Down-regulated | 226 |
| ENSG00000164161 | HHIP | 64399 | Down-regulated | 227 |
| ENSG00000100626 | GALNT16 | 57452 | Down-regulated | 228 |
| ENSG00000134874 | DZIP1 | 22873 | Down-regulated | 229 |
| ENSG00000090376 | IRAK3 | 11213 | Down-regulated | 230 |
| ENSG00000182632 | CCNYL2 | NA | Down-regulated | 231 |
| ENSG00000102383 | ZDHHC15 | 158866 | Down-regulated | 232 |
| ENSG00000144681 | STAC | 6769 | Down-regulated | 233 |
| ENSG00000137198 | GMPR | 2766 | Down-regulated | 234 |
| ENSG00000196353 | CPNE4 | 131034 | Down-regulated | 235 |
| ENSG00000162444 | RBP7 | 116362 | Down-regulated | 236 |
| ENSG00000129682 | FGF13 | 2258 | Down-regulated | 237 |
| ENSG00000166446 | CDYL2 | 124359 | Down-regulated | 238 |
| ENSG00000125148 | MT2A | 4502 | Down-regulated | 239 |
| ENSG00000141505 | ASGR1 | 432 | Down-regulated | 240 |
| ENSG00000154736 | ADAMTS5 | 11096 | Down-regulated | 241 |
| ENSG00000188582 | PAQR9 | 344838 | Down-regulated | 242 |
| ENSG00000179546 | HTR1D | 3352 | Down-regulated | 243 |
| ENSG00000113946 | CLDN16 | 10686 | Down-regulated | 244 |
| ENSG00000145362 | ANK2 | 287 | Down-regulated | 245 |
| ENSG00000155011 | DKK2 | 27123 | Down-regulated | 246 |
| ENSG00000205038 | PKHD1L1 | 93035 | Down-regulated | 247 |
| ENSG00000179954 | SSC5D | 284297 | Down-regulated | 248 |
| ENSG00000102265 | TIMP1 | 7076 | Down-regulated | 249 |
| ENSG00000156587 | UBE2L6 | 9246 | Down-regulated | 250 |
| ENSG00000167034 | NKX3-1 | 4824 | Down-regulated | 251 |
| ENSG00000184378 | ACTRT3 | 84517 | Down-regulated | 252 |
| ENSG00000081803 | CADPS2 | 93664 | Down-regulated | 253 |
| ENSG00000149131 | SERPING1 | 710 | Down-regulated | 254 |
| ENSG00000047648 | ARHGAP6 | 395 | Down-regulated | 255 |
| ENSG00000037280 | FLT4 | 2324 | Down-regulated | 256 |
| ENSG00000174672 | BRSK2 | 9024 | Down-regulated | 257 |
| ENSG00000110324 | IL10RA | 3587 | Down-regulated | 258 |
| ENSG00000164398 | ACSL6 | 23305 | Down-regulated | 259 |
| ENSG00000172936 | MYD88 | 4615 | Down-regulated | 260 |
| ENSG00000134363 | FST | 10468 | Down-regulated | 261 |
| ENSG00000103742 | IGDCC4 | 57722 | Down-regulated | 262 |
| ENSG00000116106 | EPHA4 | 2043 | Down-regulated | 263 |
| ENSG00000155961 | RAB39B | 116442 | Down-regulated | 264 |
| ENSG00000156453 | PCDH1 | 5097 | Down-regulated | 265 |
| ENSG00000148288 | GBGT1 | 26301 | Down-regulated | 266 |
| ENSG00000174307 | PHLDA3 | 23612 | Down-regulated | 267 |
| ENSG00000138131 | LOXL4 | 84171 | Down-regulated | 268 |
| ENSG00000184058 | TBX1 | 6899 | Down-regulated | 269 |
| ENSG00000050555 | LAMC3 | 10319 | Down-regulated | 270 |
| ENSG00000197410 | DCHS2 | 54798 | Down-regulated | 271 |
| ENSG00000164112 | TMEM155 | 132332 | Down-regulated | 272 |
| ENSG00000069535 | MAOB | 4129 | Down-regulated | 273 |
| ENSG00000166147 | FBN1 | 2200 | Down-regulated | 274 |
| ENSG00000042493 | CAPG | 822 | Down-regulated | 275 |
| ENSG00000075340 | ADD2 | 119 | Down-regulated | 276 |
| ENSG00000076356 | PLXNA2 | 5362 | Down-regulated | 277 |
| ENSG00000166888 | STAT6 | 6778 | Down-regulated | 278 |
| ENSG00000273274 | ZBTB8B | 728116 | Down-regulated | 279 |
| ENSG00000121316 | PLBD1 | 79887 | Down-regulated | 280 |
| ENSG00000136425 | CIB2 | 10518 | Down-regulated | 281 |
| ENSG00000151834 | GABRA2 | 2555 | Down-regulated | 282 |
| ENSG00000154330 | PGM5 | 5239 | Down-regulated | 283 |
| ENSG00000148908 | RGS10 | 6001 | Down-regulated | 284 |
| ENSG00000139970 | RTN1 | 6252 | Down-regulated | 285 |
| ENSG00000134326 | CMPK2 | 129607 | Down-regulated | 286 |
| ENSG00000100379 | KCTD17 | 79734 | Down-regulated | 287 |
| ENSG00000067798 | NAV3 | 89795 | Down-regulated | 288 |
| ENSG00000154229 | PRKCA | 5578 | Down-regulated | 289 |
| ENSG00000105255 | FSD1 | 79187 | Down-regulated | 290 |
| ENSG00000141314 | RHBDL3 | 162494 | Down-regulated | 291 |
| ENSG00000088881 | EBF4 | 57593 | Down-regulated | 292 |
| ENSG00000187902 | SHISA7 | 729956 | Down-regulated | 293 |
| ENSG00000169594 | BNC1 | 646 | Down-regulated | 294 |
| ENSG00000185950 | IRS2 | 8660 | Down-regulated | 295 |
| ENSG00000166897 | ELFN2 | 114794 | Down-regulated | 296 |
| ENSG00000171227 | TMEM37 | 140738 | Down-regulated | 297 |
| ENSG00000089327 | FXYD5 | 53827 | Down-regulated | 298 |
| ENSG00000163453 | IGFBP7 | 3490 | Down-regulated | 299 |
| ENSG00000142149 | HUNK | 30811 | Down-regulated | 300 |
| ENSG00000169783 | LINGO1 | 84894 | Down-regulated | 301 |
| ENSG00000112559 | MDFI | 4188 | Down-regulated | 302 |
| ENSG00000131094 | C1QL1 | 10882 | Down-regulated | 303 |
| ENSG00000135144 | DTX1 | 1840 | Down-regulated | 304 |
| ENSG00000147234 | FRMPD3 | 84443 | Down-regulated | 305 |
| ENSG00000162951 | LRRTM1 | 347730 | Down-regulated | 306 |
| ENSG00000189221 | MAOA | 4128 | Down-regulated | 307 |
| ENSG00000133083 | DCLK1 | 9201 | Down-regulated | 308 |
| ENSG00000162367 | TAL1 | 6886 | Down-regulated | 309 |
| ENSG00000224940 | PRRT4 | 401399 | Down-regulated | 310 |
| ENSG00000186891 | TNFRSF18 | 8784 | Down-regulated | 311 |
| ENSG00000158089 | GALNT14 | 79623 | Down-regulated | 312 |
| ENSG00000260947 | AL356489.2 | NA | Down-regulated | 313 |
| ENSG00000122025 | FLT3 | 2322 | Down-regulated | 314 |
| ENSG00000161249 | DMKN | 93099 | Down-regulated | 315 |
| ENSG00000139289 | PHLDA1 | 22822 | Down-regulated | 316 |
| ENSG00000112333 | NR2E1 | 7101 | Down-regulated | 317 |
| ENSG00000186197 | EDARADD | 128178 | Down-regulated | 318 |
| ENSG00000075461 | CACNG4 | 27092 | Down-regulated | 319 |
| ENSG00000107551 | RASSF4 | 83937 | Down-regulated | 320 |
| ENSG00000175274 | TP53I11 | 9537 | Down-regulated | 321 |
| ENSG00000163879 | DNALI1 | 7802 | Down-regulated | 322 |
| ENSG00000166250 | CLMP | 79827 | Down-regulated | 323 |
| ENSG00000101265 | RASSF2 | 9770 | Down-regulated | 324 |
| ENSG00000163531 | NFASC | 23114 | Down-regulated | 325 |
| ENSG00000091513 | TF | 7018 | Down-regulated | 326 |
| ENSG00000164318 | EGFLAM | 133584 | Down-regulated | 327 |
| ENSG00000113296 | THBS4 | 7060 | Down-regulated | 328 |
| ENSG00000172260 | NEGR1 | 257194 | Down-regulated | 329 |
| ENSG00000177807 | KCNJ10 | 3766 | Down-regulated | 330 |
| ENSG00000129159 | KCNC1 | 3746 | Down-regulated | 331 |
| ENSG00000184524 | CEND1 | 51286 | Down-regulated | 332 |
| ENSG00000154721 | JAM2 | 58494 | Down-regulated | 333 |
| ENSG00000186854 | TRABD2A | 129293 | Down-regulated | 334 |
| ENSG00000081059 | TCF7 | 6932 | Down-regulated | 335 |
| ENSG00000185155 | MIXL1 | 83881 | Down-regulated | 336 |
| ENSG00000081842 | PCDHA6 | 56142 | Down-regulated | 337 |
| ENSG00000108602 | ALDH3A1 | 218 | Down-regulated | 338 |
| ENSG00000144406 | UNC80 | 285175 | Down-regulated | 339 |
| ENSG00000006606 | CCL26 | 10344 | Down-regulated | 340 |
| ENSG00000103485 | QPRT | 105369247 | Down-regulated | 341 |
| ENSG00000103485 | QPRT | 23475 | Down-regulated | 342 |
| ENSG00000128591 | FLNC | 2318 | Down-regulated | 343 |
| ENSG00000186469 | GNG2 | 54331 | Down-regulated | 344 |
| ENSG00000213626 | LBH | 81606 | Down-regulated | 345 |
| ENSG00000163040 | CCDC74A | 90557 | Down-regulated | 346 |
| ENSG00000143786 | CNIH3 | 149111 | Down-regulated | 347 |
| ENSG00000180818 | HOXC10 | 3226 | Down-regulated | 348 |
| ENSG00000171246 | NPTX1 | 4884 | Down-regulated | 349 |
| ENSG00000164619 | BMPER | 168667 | Down-regulated | 350 |
| ENSG00000082175 | PGR | 5241 | Down-regulated | 351 |
| ENSG00000064218 | DMRT3 | 58524 | Down-regulated | 352 |
| ENSG00000167680 | SEMA6B | 10501 | Down-regulated | 353 |
| ENSG00000117152 | RGS4 | 5999 | Down-regulated | 354 |
| ENSG00000141540 | TTYH2 | 94015 | Down-regulated | 355 |
| ENSG00000159640 | ACE | 1636 | Down-regulated | 356 |
| ENSG00000146250 | PRSS35 | 167681 | Down-regulated | 357 |
| ENSG00000092096 | SLC22A17 | 51310 | Down-regulated | 358 |
| ENSG00000167311 | ART5 | 116969 | Down-regulated | 359 |
| ENSG00000177875 | CCDC184 | 387856 | Down-regulated | 360 |
| ENSG00000104267 | CA2 | 760 | Down-regulated | 361 |
| ENSG00000250510 | GPR162 | 27239 | Down-regulated | 362 |
| ENSG00000244509 | APOBEC3C | 27350 | Down-regulated | 363 |
| ENSG00000115232 | ITGA4 | 3676 | Down-regulated | 364 |
| ENSG00000171282 | NA | NA | Down-regulated | 365 |
| ENSG00000067840 | PDZD4 | 57595 | Down-regulated | 366 |
| ENSG00000112246 | SIM1 | 6492 | Down-regulated | 367 |
| ENSG00000164342 | TLR3 | 7098 | Down-regulated | 368 |
| ENSG00000272695 | GAS6-DT | NA | Down-regulated | 369 |
| ENSG00000019186 | CYP24A1 | 1591 | Down-regulated | 370 |
| ENSG00000111728 | ST8SIA1 | 6489 | Down-regulated | 371 |
| ENSG00000138622 | HCN4 | 10021 | Down-regulated | 372 |
| ENSG00000119865 | CNRIP1 | 25927 | Down-regulated | 373 |
| ENSG00000197261 | C6orf141 | 135398 | Down-regulated | 374 |
| ENSG00000115380 | EFEMP1 | 2202 | Down-regulated | 375 |
| ENSG00000176697 | BDNF | 627 | Down-regulated | 376 |
| ENSG00000134013 | LOXL2 | 4017 | Down-regulated | 377 |
| ENSG00000169554 | ZEB2 | 9839 | Down-regulated | 378 |
| ENSG00000131620 | ANO1 | 55107 | Down-regulated | 379 |
| ENSG00000227825 | SLC9A7P1 | NA | Down-regulated | 380 |
| ENSG00000084628 | NKAIN1 | 79570 | Down-regulated | 381 |
| ENSG00000084636 | COL16A1 | 1307 | Down-regulated | 382 |
| ENSG00000116983 | HPCAL4 | 51440 | Down-regulated | 383 |
| ENSG00000235387 | SPAAR | 158376 | Down-regulated | 384 |
| ENSG00000078596 | ITM2A | 9452 | Down-regulated | 385 |
| ENSG00000068831 | RASGRP2 | 10235 | Down-regulated | 386 |
| ENSG00000139874 | SSTR1 | 6751 | Down-regulated | 387 |
| ENSG00000137727 | ARHGAP20 | 57569 | Down-regulated | 388 |
| ENSG00000259070 | LINC00639 | NA | Down-regulated | 389 |
| ENSG00000159713 | TPPP3 | 51673 | Down-regulated | 390 |
| ENSG00000164929 | BAALC | 79870 | Down-regulated | 391 |
| ENSG00000079102 | RUNX1T1 | 862 | Down-regulated | 392 |
| ENSG00000196104 | SPOCK3 | 50859 | Down-regulated | 393 |
| ENSG00000168394 | TAP1 | 6890 | Down-regulated | 394 |
| ENSG00000188452 | CERKL | 375298 | Down-regulated | 395 |
| ENSG00000153071 | DAB2 | 1601 | Down-regulated | 396 |
| ENSG00000171811 | CFAP46 | 54777 | Down-regulated | 397 |
| ENSG00000111186 | WNT5B | 81029 | Down-regulated | 398 |
| ENSG00000172935 | MRGPRF | 116535 | Down-regulated | 399 |
| ENSG00000166448 | TMEM130 | 222865 | Down-regulated | 400 |
| ENSG00000147255 | IGSF1 | 3547 | Down-regulated | 401 |
| ENSG00000176406 | RIMS2 | 9699 | Down-regulated | 402 |
| ENSG00000162373 | BEND5 | 79656 | Down-regulated | 403 |
| ENSG00000134775 | FHOD3 | 80206 | Down-regulated | 404 |
| ENSG00000004809 | SLC22A16 | 85413 | Down-regulated | 405 |
| ENSG00000148408 | CACNA1B | 774 | Down-regulated | 406 |
| ENSG00000141526 | SLC16A3 | 9123 | Down-regulated | 407 |
| ENSG00000117598 | PLPPR5 | 163404 | Down-regulated | 408 |
| ENSG00000105642 | KCNN1 | 3780 | Down-regulated | 409 |
| ENSG00000050438 | SLC4A8 | 9498 | Down-regulated | 410 |
| ENSG00000116771 | AGMAT | 79814 | Down-regulated | 411 |
| ENSG00000229373 | LINC00452 | 643365 | Down-regulated | 412 |
| ENSG00000223865 | HLA-DPB1 | 3115 | Down-regulated | 413 |
| ENSG00000224818 | AC096677.2 | NA | Down-regulated | 414 |
| ENSG00000123342 | MMP19 | 4327 | Down-regulated | 415 |
| ENSG00000002726 | AOC1 | 26 | Down-regulated | 416 |
| ENSG00000140479 | PCSK6 | 5046 | Down-regulated | 417 |
| ENSG00000223477 | NA | NA | Down-regulated | 418 |
| ENSG00000225968 | ELFN1 | 392617 | Down-regulated | 419 |
| ENSG00000137273 | FOXF2 | 2295 | Down-regulated | 420 |
| ENSG00000134070 | IRAK2 | 3656 | Down-regulated | 421 |
| ENSG00000148948 | LRRC4C | 57689 | Down-regulated | 422 |
| ENSG00000235217 | TSPY26P | NA | Down-regulated | 423 |
| ENSG00000182255 | KCNA4 | 3739 | Down-regulated | 424 |
| ENSG00000121753 | ADGRB2 | 576 | Down-regulated | 425 |
| ENSG00000118946 | PCDH17 | 27253 | Down-regulated | 426 |
| ENSG00000163377 | FAM19A4 | 151647 | Down-regulated | 427 |
| ENSG00000172733 | PURG | 29942 | Down-regulated | 428 |
| ENSG00000205363 | C15orf59 | 388135 | Down-regulated | 429 |
| ENSG00000267121 | AC008105.3 | NA | Down-regulated | 430 |
| ENSG00000172020 | GAP43 | 2596 | Down-regulated | 431 |
| ENSG00000204970 | PCDHA1 | 56147 | Down-regulated | 432 |
| ENSG00000157150 | TIMP4 | 7079 | Down-regulated | 433 |
| ENSG00000005243 | COPZ2 | 51226 | Down-regulated | 434 |
| ENSG00000134802 | SLC43A3 | 29015 | Down-regulated | 435 |
| ENSG00000144339 | TMEFF2 | 23671 | Down-regulated | 436 |
| ENSG00000149403 | GRIK4 | 2900 | Down-regulated | 437 |
| ENSG00000078053 | AMPH | 273 | Down-regulated | 438 |
| ENSG00000123364 | HOXC13 | 3229 | Down-regulated | 439 |
| ENSG00000162545 | CAMK2N1 | 55450 | Down-regulated | 440 |
| ENSG00000188848 | BEND4 | 389206 | Down-regulated | 441 |
| ENSG00000104518 | GSDMD | 79792 | Down-regulated | 442 |
| ENSG00000152932 | RAB3C | 115827 | Down-regulated | 443 |
| ENSG00000183798 | EMILIN3 | 90187 | Down-regulated | 444 |
| ENSG00000105711 | SCN1B | 6324 | Down-regulated | 445 |
| ENSG00000183671 | GPR1 | 2825 | Down-regulated | 446 |
| ENSG00000107105 | ELAVL2 | 1993 | Down-regulated | 447 |
| ENSG00000106624 | AEBP1 | 165 | Down-regulated | 448 |
| ENSG00000126259 | KIRREL2 | 84063 | Down-regulated | 449 |
| ENSG00000168280 | KIF5C | 3800 | Down-regulated | 450 |
| ENSG00000157152 | SYN2 | 6854 | Down-regulated | 451 |
| ENSG00000113389 | NPR3 | 4883 | Down-regulated | 452 |
| ENSG00000100060 | MFNG | 4242 | Down-regulated | 453 |
| ENSG00000163762 | TM4SF18 | 116441 | Down-regulated | 454 |
| ENSG00000177359 | AC024940.2 | NA | Down-regulated | 455 |
| ENSG00000203883 | SOX18 | 54345 | Down-regulated | 456 |
| ENSG00000148516 | ZEB1 | 6935 | Down-regulated | 457 |
| ENSG00000272636 | DOC2B | 8447 | Down-regulated | 458 |
| ENSG00000165633 | VSTM4 | 196740 | Down-regulated | 459 |
| ENSG00000196549 | MME | 4311 | Down-regulated | 460 |
| ENSG00000235098 | ANKRD65 | 441869 | Down-regulated | 461 |
| ENSG00000267123 | LINC02081 | NA | Down-regulated | 462 |
| ENSG00000137672 | TRPC6 | 7225 | Down-regulated | 463 |
| ENSG00000233384 | AC096537.1 | NA | Down-regulated | 464 |
| ENSG00000092051 | JPH4 | 84502 | Down-regulated | 465 |
| ENSG00000174348 | PODN | 127435 | Down-regulated | 466 |
| ENSG00000184905 | TCEAL2 | 140597 | Down-regulated | 467 |
| ENSG00000011422 | PLAUR | 5329 | Down-regulated | 468 |
| ENSG00000249158 | PCDHA11 | 56138 | Down-regulated | 469 |
| ENSG00000230453 | ANKRD18B | 441459 | Down-regulated | 470 |
| ENSG00000120149 | MSX2 | 4488 | Down-regulated | 471 |
| ENSG00000110328 | GALNT18 | 374378 | Down-regulated | 472 |
| ENSG00000154319 | FAM167A | 83648 | Down-regulated | 473 |
| ENSG00000186907 | RTN4RL2 | 349667 | Down-regulated | 474 |
| ENSG00000167600 | CYP2S1 | 29785 | Down-regulated | 475 |
| ENSG00000169071 | ROR2 | 4920 | Down-regulated | 476 |
| ENSG00000261786 | AC006058.1 | NA | Down-regulated | 477 |
| ENSG00000137101 | CD72 | 971 | Down-regulated | 478 |
| ENSG00000170162 | VGLL2 | 245806 | Down-regulated | 479 |
| ENSG00000184809 | B3GALT5-AS1 | NA | Down-regulated | 480 |
| ENSG00000204267 | TAP2 | 6891 | Down-regulated | 481 |
| ENSG00000151702 | FLI1 | 2313 | Down-regulated | 482 |
| ENSG00000169083 | AR | 367 | Down-regulated | 483 |
| ENSG00000266278 | LINC01910 | NA | Down-regulated | 484 |
| ENSG00000165323 | FAT3 | 120114 | Down-regulated | 485 |
| ENSG00000145934 | TENM2 | 57451 | Down-regulated | 486 |
| ENSG00000146070 | PLA2G7 | 7941 | Down-regulated | 487 |
| ENSG00000136944 | LMX1B | 4010 | Down-regulated | 488 |
| ENSG00000101282 | RSPO4 | 343637 | Down-regulated | 489 |
| ENSG00000159212 | CLIC6 | 54102 | Down-regulated | 490 |
| ENSG00000155816 | FMN2 | 56776 | Down-regulated | 491 |
| ENSG00000188620 | HMX3 | 340784 | Down-regulated | 492 |
| ENSG00000180806 | HOXC9 | 3225 | Down-regulated | 493 |
| ENSG00000107984 | DKK1 | 22943 | Down-regulated | 494 |
| ENSG00000166426 | CRABP1 | 1381 | Down-regulated | 495 |
| ENSG00000172548 | NIPAL4 | 348938 | Down-regulated | 496 |
| ENSG00000142227 | EMP3 | 2014 | Down-regulated | 497 |
| ENSG00000167779 | IGFBP6 | 3489 | Down-regulated | 498 |
| ENSG00000151490 | PTPRO | 5800 | Down-regulated | 499 |
| ENSG00000162105 | SHANK2 | 22941 | Down-regulated | 500 |
| ENSG00000034239 | EFCAB1 | 79645 | Down-regulated | 501 |
| ENSG00000173376 | NDNF | 79625 | Down-regulated | 502 |
| ENSG00000102755 | FLT1 | 2321 | Down-regulated | 503 |
| ENSG00000091986 | CCDC80 | 151887 | Down-regulated | 504 |
| ENSG00000261115 | TMEM178B | 100507421 | Down-regulated | 505 |
| ENSG00000267102 | AC060766.1 | NA | Down-regulated | 506 |
| ENSG00000178150 | ZNF114 | 163071 | Down-regulated | 507 |
| ENSG00000128340 | RAC2 | 5880 | Down-regulated | 508 |
| ENSG00000170801 | HTRA3 | 94031 | Down-regulated | 509 |
| ENSG00000179603 | GRM8 | 2918 | Down-regulated | 510 |
| ENSG00000139219 | COL2A1 | 1280 | Down-regulated | 511 |
| ENSG00000077942 | FBLN1 | 2192 | Down-regulated | 512 |
| ENSG00000165349 | SLC7A3 | 84889 | Down-regulated | 513 |
| ENSG00000163283 | ALPP | 250 | Down-regulated | 514 |
| ENSG00000170989 | S1PR1 | 1901 | Down-regulated | 515 |
| ENSG00000000971 | CFH | 3075 | Down-regulated | 516 |
| ENSG00000259886 | NA | NA | Down-regulated | 517 |
| ENSG00000172985 | SH3RF3 | 344558 | Down-regulated | 518 |
| ENSG00000118432 | CNR1 | 1268 | Down-regulated | 519 |
| ENSG00000240694 | PNMA2 | 10687 | Down-regulated | 520 |
| ENSG00000168676 | KCTD19 | 146212 | Down-regulated | 521 |
| ENSG00000177464 | GPR4 | 2828 | Down-regulated | 522 |
| ENSG00000160801 | PTH1R | 5745 | Down-regulated | 523 |
| ENSG00000101134 | DOK5 | 55816 | Down-regulated | 524 |
| ENSG00000148704 | VAX1 | 11023 | Down-regulated | 525 |
| ENSG00000236914 | LINC01852 | NA | Down-regulated | 526 |
| ENSG00000188133 | TMEM215 | 401498 | Down-regulated | 527 |
| ENSG00000269993 | KC877982.1 | NA | Down-regulated | 528 |
| ENSG00000049247 | UTS2 | 10911 | Down-regulated | 529 |
| ENSG00000198053 | SIRPA | 140885 | Down-regulated | 530 |
| ENSG00000123360 | PDE1B | 5153 | Down-regulated | 531 |
| ENSG00000164694 | FNDC1 | 84624 | Down-regulated | 532 |
| ENSG00000102195 | GPR50 | 9248 | Down-regulated | 533 |
| ENSG00000183807 | FAM162B | 221303 | Down-regulated | 534 |
| ENSG00000130038 | CRACR2A | 84766 | Down-regulated | 535 |
| ENSG00000101210 | EEF1A2 | 1917 | Down-regulated | 536 |
| ENSG00000272761 | NA | NA | Down-regulated | 537 |
| ENSG00000250056 | LINC01018 | NA | Down-regulated | 538 |
| ENSG00000184371 | CSF1 | 1435 | Down-regulated | 539 |
| ENSG00000037965 | HOXC8 | 3224 | Down-regulated | 540 |
| ENSG00000024422 | EHD2 | 30846 | Down-regulated | 541 |
| ENSG00000086991 | NOX4 | 50507 | Down-regulated | 542 |
| ENSG00000129009 | ISLR | 3671 | Down-regulated | 543 |
| ENSG00000250320 | AC113383.1 | NA | Down-regulated | 544 |
| ENSG00000105696 | TMEM59L | 25789 | Down-regulated | 545 |
| ENSG00000157851 | DPYSL5 | 56896 | Down-regulated | 546 |
| ENSG00000125378 | BMP4 | 652 | Down-regulated | 547 |
| ENSG00000106483 | SFRP4 | 6424 | Down-regulated | 548 |
| ENSG00000136352 | NKX2-1 | 7080 | Down-regulated | 549 |
| ENSG00000144810 | COL8A1 | 1295 | Down-regulated | 550 |
| ENSG00000170961 | HAS2 | 3037 | Down-regulated | 551 |
| ENSG00000102452 | NALCN | 259232 | Down-regulated | 552 |
| ENSG00000250742 | LINC02381 | NA | Down-regulated | 553 |
| ENSG00000151778 | SERP2 | 387923 | Down-regulated | 554 |
| ENSG00000132932 | ATP8A2 | 51761 | Down-regulated | 555 |
| ENSG00000138685 | FGF2 | 2247 | Down-regulated | 556 |
| ENSG00000151640 | DPYSL4 | 10570 | Down-regulated | 557 |
| ENSG00000225206 | MIR137HG | NA | Down-regulated | 558 |
| ENSG00000128342 | LIF | 3976 | Down-regulated | 559 |
| ENSG00000128918 | ALDH1A2 | 8854 | Down-regulated | 560 |
| ENSG00000168404 | MLKL | 197259 | Down-regulated | 561 |
| ENSG00000137726 | FXYD6 | 53826 | Down-regulated | 562 |
| ENSG00000253304 | TMEM200B | 399474 | Down-regulated | 563 |
| ENSG00000141150 | NA | NA | Down-regulated | 564 |
| ENSG00000006071 | ABCC8 | 6833 | Down-regulated | 565 |
| ENSG00000006638 | TBXA2R | 6915 | Down-regulated | 566 |
| ENSG00000132329 | RAMP1 | 10267 | Down-regulated | 567 |
| ENSG00000182107 | TMEM30B | 161291 | Down-regulated | 568 |
| ENSG00000110076 | NRXN2 | 9379 | Down-regulated | 569 |
| ENSG00000227039 | ITGB2-AS1 | NA | Down-regulated | 570 |
| ENSG00000232480 | TGFB2-AS1 | NA | Down-regulated | 571 |
| ENSG00000104332 | SFRP1 | 6422 | Down-regulated | 572 |
| ENSG00000112902 | SEMA5A | 9037 | Down-regulated | 573 |
| ENSG00000139200 | PIANP | 196500 | Down-regulated | 574 |
| ENSG00000196639 | HRH1 | 3269 | Down-regulated | 575 |
| ENSG00000011028 | MRC2 | 9902 | Down-regulated | 576 |
| ENSG00000107807 | TLX1 | 3195 | Down-regulated | 577 |
| ENSG00000128268 | MGAT3 | 4248 | Down-regulated | 578 |
| ENSG00000007038 | PRSS21 | 10942 | Down-regulated | 579 |
| ENSG00000204442 | FAM155A | 728215 | Down-regulated | 580 |
| ENSG00000225614 | ZNF469 | 84627 | Down-regulated | 581 |
| ENSG00000155970 | MICU3 | 286097 | Down-regulated | 582 |
| ENSG00000122420 | PTGFR | 5737 | Down-regulated | 583 |
| ENSG00000204362 | AL590644.1 | NA | Down-regulated | 584 |
| ENSG00000143631 | FLG | 2312 | Down-regulated | 585 |
| ENSG00000059804 | SLC2A3 | 6515 | Down-regulated | 586 |
| ENSG00000107518 | ATRNL1 | 26033 | Down-regulated | 587 |
| ENSG00000115648 | MLPH | 79083 | Down-regulated | 588 |
| ENSG00000087245 | MMP2 | 4313 | Down-regulated | 589 |
| ENSG00000074047 | GLI2 | 2736 | Down-regulated | 590 |
| ENSG00000131435 | PDLIM4 | 8572 | Down-regulated | 591 |
| ENSG00000145681 | HAPLN1 | 1404 | Down-regulated | 592 |
| ENSG00000179855 | GIPC3 | 126326 | Down-regulated | 593 |
| ENSG00000204381 | LAYN | 143903 | Down-regulated | 594 |
| ENSG00000143473 | KCNH1 | 3756 | Down-regulated | 595 |
| ENSG00000117069 | ST6GALNAC5 | 81849 | Down-regulated | 596 |
| ENSG00000134259 | NGF | 4803 | Down-regulated | 597 |
| ENSG00000147402 | NA | NA | Down-regulated | 598 |
| ENSG00000104967 | NOVA2 | 4858 | Down-regulated | 599 |
| ENSG00000151743 | AMN1 | 196394 | Down-regulated | 600 |
| ENSG00000013297 | CLDN11 | 5010 | Down-regulated | 601 |
| ENSG00000178882 | RFLNA | 100533183 | Down-regulated | 602 |
| ENSG00000178882 | RFLNA | 144347 | Down-regulated | 603 |
| ENSG00000175445 | LPL | 4023 | Down-regulated | 604 |
| ENSG00000183580 | FBXL7 | 23194 | Down-regulated | 605 |
| ENSG00000006611 | USH1C | 10083 | Down-regulated | 606 |
| ENSG00000158352 | SHROOM4 | 57477 | Down-regulated | 607 |
| ENSG00000144583 | Mar-04 | 57574 | Down-regulated | 608 |
| ENSG00000101445 | PPP1R16B | 26051 | Down-regulated | 609 |
| ENSG00000163739 | CXCL1 | 2919 | Down-regulated | 610 |
| ENSG00000104833 | TUBB4A | 10382 | Down-regulated | 611 |
| ENSG00000149557 | FEZ1 | 9638 | Down-regulated | 612 |
| ENSG00000138496 | PARP9 | 83666 | Down-regulated | 613 |
| ENSG00000172123 | SLFN12 | 55106 | Down-regulated | 614 |
| ENSG00000163840 | DTX3L | 151636 | Down-regulated | 615 |
| ENSG00000158445 | KCNB1 | 3745 | Down-regulated | 616 |
| ENSG00000122824 | NUDT10 | 170685 | Down-regulated | 617 |
| ENSG00000179299 | NSUN7 | 79730 | Down-regulated | 618 |
| ENSG00000125355 | TMEM255A | 55026 | Down-regulated | 619 |
| ENSG00000154783 | FGD5 | 152273 | Down-regulated | 620 |
| ENSG00000197757 | HOXC6 | 3223 | Down-regulated | 621 |
| ENSG00000164176 | EDIL3 | 10085 | Down-regulated | 622 |
| ENSG00000167178 | ISLR2 | 57611 | Down-regulated | 623 |
| ENSG00000183876 | ARSI | 340075 | Down-regulated | 624 |
| ENSG00000078549 | ADCYAP1R1 | 117 | Down-regulated | 625 |
| ENSG00000139364 | TMEM132B | 114795 | Down-regulated | 626 |
| ENSG00000166825 | ANPEP | 290 | Down-regulated | 627 |
| ENSG00000154678 | PDE1C | 5137 | Down-regulated | 628 |
| ENSG00000144218 | AFF3 | 3899 | Down-regulated | 629 |
| ENSG00000105409 | ATP1A3 | 478 | Down-regulated | 630 |
| ENSG00000135447 | PPP1R1A | 5502 | Down-regulated | 631 |
| ENSG00000026025 | VIM | 7431 | Down-regulated | 632 |
| ENSG00000122641 | INHBA | 3624 | Down-regulated | 633 |
| ENSG00000176049 | JAKMIP2 | 9832 | Down-regulated | 634 |
| ENSG00000141753 | IGFBP4 | 3487 | Down-regulated | 635 |
| ENSG00000171551 | ECEL1 | 9427 | Down-regulated | 636 |
| ENSG00000115361 | ACADL | 33 | Down-regulated | 637 |
| ENSG00000170571 | EMB | 133418 | Down-regulated | 638 |
| ENSG00000260549 | MT1L | 4500 | Down-regulated | 639 |
| ENSG00000106571 | GLI3 | 2737 | Down-regulated | 640 |
| ENSG00000123609 | NMI | 9111 | Down-regulated | 641 |
| ENSG00000165140 | FBP1 | 2203 | Down-regulated | 642 |
| ENSG00000038427 | VCAN | 1462 | Down-regulated | 643 |
| ENSG00000146938 | NLGN4X | 57502 | Down-regulated | 644 |
| ENSG00000237515 | SHISA9 | 729993 | Down-regulated | 645 |
| ENSG00000019549 | SNAI2 | 6591 | Down-regulated | 646 |
| ENSG00000067177 | PHKA1 | 5255 | Down-regulated | 647 |
| ENSG00000183778 | B3GALT5 | 10317 | Down-regulated | 648 |
| ENSG00000168671 | UGT3A2 | 167127 | Down-regulated | 649 |
| ENSG00000130635 | COL5A1 | 1289 | Down-regulated | 650 |
| ENSG00000169515 | CCDC8 | 83987 | Down-regulated | 651 |
| ENSG00000116132 | PRRX1 | 5396 | Down-regulated | 652 |
| ENSG00000086696 | HSD17B2 | 3294 | Down-regulated | 653 |
| ENSG00000131089 | ARHGEF9 | 23229 | Down-regulated | 654 |
| ENSG00000169181 | GSG1L | 146395 | Down-regulated | 655 |
| ENSG00000167601 | AXL | 558 | Down-regulated | 656 |
| ENSG00000140945 | CDH13 | 1012 | Down-regulated | 657 |
| ENSG00000151892 | GFRA1 | 2674 | Down-regulated | 658 |
| ENSG00000152092 | ASTN1 | 460 | Down-regulated | 659 |

These differentially expressed genes (DEGs) were found to be enriched in 20 molecular pathways by the KEGG pathway enrichment analysis (Table 4), with more genes down-regulated rather than up-regulated in these pathways. Pathways that may be relevant to embryo implantation include ECR-receptor interaction, cell adhesion, focal adhesion and signalling of calcium, Wnt and cAMP and leukocyte transendothelial migration (Table 4).

TABLE 4
Molecular pathways enriched by differentially expressed genes

| | Number of | | | | |
|---|---|---|---|---|---|
| | genes altered | | | Gene names | |
| Enriched Pathways | Total | Up | Down | Up | Down |
| Mucin type O-glycan biosynthesis | 9 | 4 | 5 | GALNT3 | GALNT14 |
| | | | | GCNT3 | GALNT18 |
| | | | | ST6GALNAC1 | GALNT6 |
| | | | | GALNT13 | GALNT16 |
| | | | | | GALNT5 |
| Morphine addiction | 15 | 4 | 11 | GABRP | PRKCA |
| | | | | ADCY5 | PDE1C |
| | | | | ARRB1 | GNG2 |
| | | | | KCNJ5 | PDE1B |
| | | | | | CACNA1B |
| | | | | | GNG4 |
| | | | | | PDE4D |
| | | | | | PDE7B |
| | | | | | KCNJ3 |
| | | | | | GABRG2 |
| | | | | | GABRA2 |
| ECM-receptor interaction | 14 | 6 | 8 | LAMB3 | ITGA3 |
| | | | | ITGB8 | THBS4 |
| | | | | ITGB6 | SV2A |
| | | | | TNC | ITGA4 |
| | | | | SPP1 | COL2A1 |
| | | | | SV2C | ITGA7 |
| | | | | | LAMC3 |
| | | | | | ITGA2B |
| Cell adhesion molecules (CAMs) | 19 | 6 | 13 | CDH3 | VCAN |
| | | | | CDH1 | NLGN4X |
| | | | | CLDN7 | CLDN11 |
| | | | | CLDN4 | NEGRI |
| | | | | ITGB8 | ITGA4 |
| | | | | NCAM2 | NFASC |
| | | | | | JAM2 |
| | | | | | LRRC4C |
| | | | | | HLA-C |
| | | | | | NRXN2 |
| | | | | | HLA-DPB1 |
| | | | | | CNTNAP1 |
| | | | | | CLDN16 |
| Hypertrophic cardiomyopathy (HCM) | 13 | 5 | 8 | ITGB8 | TGFB1 |
| | | | | EDN1 | ITGA3 |
| | | | | ITGB6 | TGFB2 |
| | | | | TNNC1 | ITGA4 |
| | | | | RYR2 | ACE |
| | | | | | ITGA7 |
| | | | | | CACNG4 |
| | | | | | ITGA2B |
| PI3K-Akt signaling pathway | 31 | 9 | 22 | SYK | PRKCA |
| | | | | LAMB3 | FGF2 |
| | | | | FGF9 | ITGA3 |
| | | | | ITGB8 | NTF3 |
| | | | | EFNA5 | THBS4 |
| | | | | ITGB6 | BDNF |
| | | | | TNC | NGF |
| | | | | SPP1 | ITGA4 |
| | | | | LPAR6 | GNG2 |
| | | | | | FLT1 |
| | | | | | CSF1 |
| | | | | | COL2A1 |
| | | | | | PPP2R2C |
| | | | | | FGFR2 |
| | | | | | GNG4 |
| | | | | | FLT4 |
| | | | | | ERBB4 |
| | | | | | ITGA7 |
| | | | | | LAMC3 |
| | | | | | IL7R |
| | | | | | FLT3 |
| | | | | | ITGA2B |
| Pathways in cancer | 41 | 10 | 31 | CDH1 | PRKCA |
| | | | | LAMB3 | FGF2 |
| | | | | WNT7A | STAT6 |
| | | | | WNT5A | TGFB1 |
| | | | | FGF9 | ITGA3 |
| | | | | ADCY5 | TGFB2 |
| | | | | PTGER3 | HHIP |
| | | | | EDN1 | GLI3 |
| | | | | LPAR6 | BMP4 |
| | | | | WNT9A | AR |
| | | | | | TCF7 |
| | | | | | GNG2 |
| | | | | | RAC2 |
| | | | | | NKX3-1 |
| | | | | | WNT5B |
| | | | | | CASP7 |
| | | | | | STAT5A |
| | | | | | GLI2 |
| | | | | | FGFR2 |
| | | | | | HEYL |
| | | | | | GNG4 |
| | | | | | RASGRP2 |
| | | | | | FLT4 |
| | | | | | CTNNA2 |
| | | | | | MMP2 |
| | | | | | LAMC3 |
| | | | | | IL7R |
| | | | | | FLT3 |
| | | | | | EDNRA |
| | | | | | ITGA2B |
| | | | | | RUNX1T1 |
| Focal adhesion | 20 | 6 | 14 | LAMB3 | PRKCA |
| | | | | ITGB8 | FLNC |
| | | | | FYN | ITGA3 |
| | | | | ITGB6 | THBS4 |
| | | | | TNC | CAV1 |
| | | | | SPP1 | MYLK |
| | | | | | ITGA4 |
| | | | | | RAC2 |
| | | | | | FLT1 |
| | | | | | COL2A1 |
| | | | | | FLT4 |
| | | | | | ITGA7 |
| | | | | | LAMC3 |
| | | | | | ITGA2B |
| Dilated cardiomyopathy (DCM) | 12 | 5 | 7 | ADCY5 | TGFB1 |
| | | | | ITGB8 | ITGA3 |
| | | | | ITGB6 | TGFB2 |
| | | | | TNNC1 | ITGA4 |
| | | | | RYR2 | ITGA7 |
| | | | | | CACNG4 |
| | | | | | ITGA2B |
| Calcium signaling pathway | 19 | 6 | 13 | GRM1 | PRKCA |
| | | | | PTGER3 | PDE1C |
| | | | | CACNA1H | PHKA1 |
| | | | | CD38 | CAMK4 |
| | | | | TNNC1 | PTGFR |
| | | | | RYR2 | MYLK |
| | | | | | ATP2A3 |
| | | | | | TBXA2R |
| | | | | | PDE1B |
| | | | | | HRH1 |
| | | | | | CACNA1B |
| | | | | | ERBB4 |
| | | | | | EDNRA |
| Axon guidance | 18 | 5 | 13 | EPHA1 | PRKCA |
| | | | | SEMA4A | SEMA5A |
| | | | | WNT5A | DPYSL2 |
| | | | | EFNA5 | SEMA3D |
| | | | | FYN | SEMA6B |
| | | | | | NGEF |
| | | | | | RAC2 |
| | | | | | EPHA4 |
| | | | | | WNT5B |
| | | | | | LRRC4C |
| | | | | | DPYSL5 |
| | | | | | TRPC6 |
| | | | | | PLXNA2 |
| Arrhythmogenic right ventricular | 10 | 3 | 7 | ITGB8 | ITGA3 |
| cardiomyopathy (ARVC) | | | | ITGB6 | TCF7 |
| | | | | RYR2 | ITGA4 |
| | | | | | ITGA7 |
| | | | | | CTNNA2 |
| | | | | | CACNG4 |
| | | | | | ITGA2B |
| Wnt signaling pathway | 16 | 5 | 11 | RSPO3 | PRKCA |
| | | | | WNT7A | SFRP1 |
| | | | | LGR4 | SFRP4 |
| | | | | WNT5A | TCF7 |
| | | | | WNT9A | DKK2 |
| | | | | | RAC2 |
| | | | | | ROR2 |
| | | | | | FOSL1 |
| | | | | | WNT5B |
| | | | | | RSPO4 |
| | | | | | DKK1 |
| Basal cell carcinoma | 9 | 3 | 6 | WNT7A | HHIP |
| | | | | WNT5A | GLI3 |
| | | | | WNT9A | BMP4 |
| | | | | | TCF7 |
| | | | | | WNT5B |
| | | | | | GLI2 |
| cAMP signaling pathway | 19 | 5 | 14 | ADCY5 | ADCYAP1R1 |
| | | | | PTGER3 | HHIP |
| | | | | EDN1 | GLI3 |
| | | | | RYR2 | CAMK4 |
| | | | | AFDN | BDNF |
| | | | | | TIAM1 |
| | | | | | RAC2 |
| | | | | | ATP1A3 |
| | | | | | HTR1D |
| | | | | | SSTR1 |
| | | | | | PDE4D |
| | | | | | HCN4 |
| | | | | | ATP1B2 |
| | | | | | EDNRA |
| Histidine metabolism | 5 | 0 | 5 | | ALDH3A1 |
| | | | | | MAOB |
| | | | | | MAOA |
| | | | | | AOC1 |
| | | | | | ALDH3B1 |
| MAPK signaling pathway | 24 | 4 | 20 | FGF9 | PRKCA |
| | | | | ARRB1 | FGF2 |
| | | | | EFNA5 | TGFB1 |
| | | | | CACNA1H | FLNC |
| | | | | | MAP3K14 |
| | | | | | TGFB2 |
| | | | | | MYD88 |
| | | | | | NTF3 |
| | | | | | BDNF |
| | | | | | NGF |
| | | | | | RAC2 |
| | | | | | FLT1 |
| | | | | | CSF1 |
| | | | | | FGFR2 |
| | | | | | CACNA1B |
| | | | | | RASGRP2 |
| | | | | | FLT4 |
| | | | | | ERBB4 |
| | | | | | FLT3 |
| | | | | | CACNG4 |
| Hematopoietic cell lineage | 11 | 2 | 9 | IL1R2 | ITGA3 |
| | | | | CD38 | MME |
| | | | | | ITGA4 |
| | | | | | CSF1 |
| | | | | | HLA-DPB1 |
| | | | | | ANPEP |
| | | | | | IL7R |
| | | | | | FLT3 |
| | | | | | ITGA2B |
| Leukocyte transendothelial | 12 | 4 | 8 | CLDN7 | PRKCA |
| migration | | | | CLDN4 | CLDN11 |
| | | | | CYBB | ITGA4 |
| | | | | AFDN | RAC2 |
| | | | | | JAM2 |
| | | | | | CTNNA2 |
| | | | | | MMP2 |
| | | | | | CLDN16 |
| Insulin secretion | 10 | 3 | 7 | ADCY5 | PRKCA |
| | | | | RYR2 | ADCYAP1R1 |
| | | | | KCNN3 | ABCC8 |
| | | | | | ATP1A3 |
| | | | | | KCNN1 |
| | | | | | RIMS2 |
| | | | | | ATP1B2 |

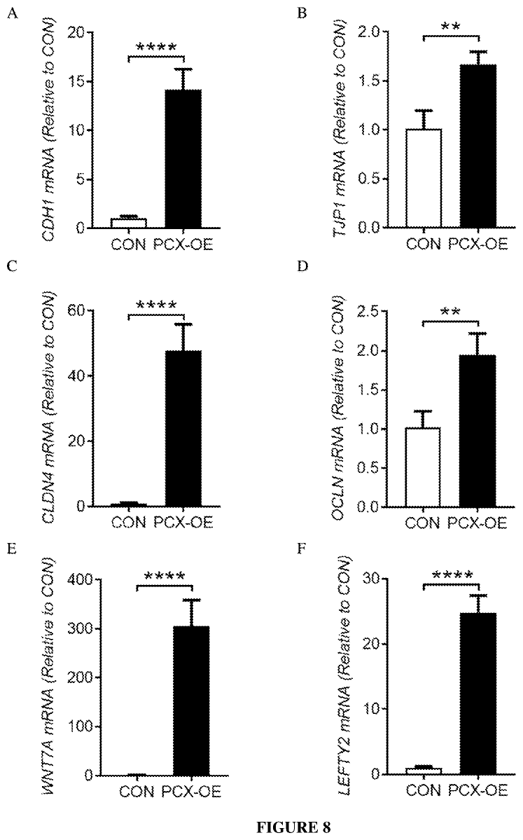

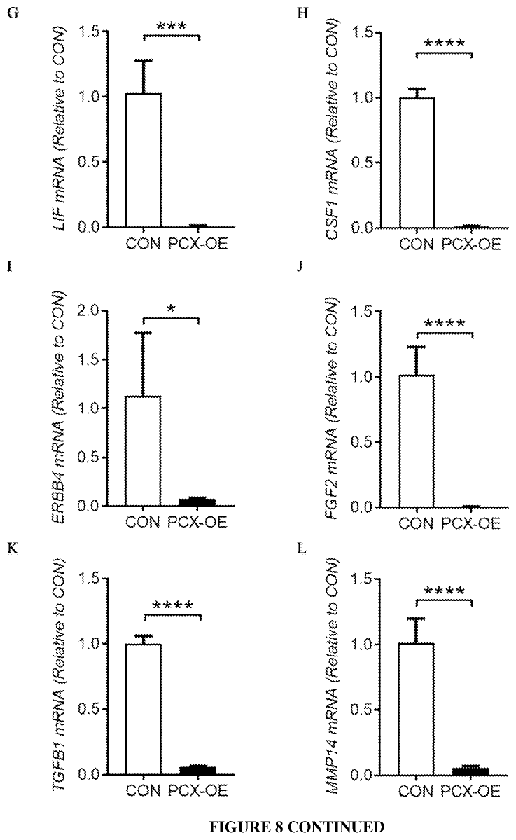

As cell adhesion and epithelial junctions are particularly important for embryo attachment and invasion, more-focused analysis of these pathways was performed. For cell adhesion related genes, 59 were differentially expressed, with 41 (70%) down- and 18 (30%) up-regulated. For epithelial tight junction, 46 genes showed differential expression, with 20 (43%) down- and 26 (57%) up-regulated. For adherence junction, 32 genes were expressed differentially, 12 (37%) down- and 20 (63%) up-regulated. For gap junction, 36 displayed differential expression, 26 (72%) down- and 10 (28%) up-regulated. Collectively, these data indicate that PCX-OE preferentially reduced expression of genes involved in cell adhesion and gap junction but increased those associated with tight/adherence junctions. In particularly, major adherence junction gene CDH1 (encoding E-cadherin), tight junction genes TJP1 (ZO-1), CLDN4 (claudin 4) and OCLN (occludin), were all significantly up-regulated in PCX-OE than control cells, which was further validated by real-time RT-PCR analysis (FIG. 8).

DEGs were further investigated to identify those that are known to be relevant to embryo implantation. As shown in FIG. 8A-F, a number of genes whose expression is linked to implantation failure, such as WNT7A (Wnt family member 7A, Wnt 7A) and LEFTY2 (left-right determination factor 2), were highly significantly up-regulated in PCX-OE cells. In contrast, a number of receptivity promoting factors, including LIF (interleukin 6 family cytokine), CSF1 (colony stimulating factor 1), ERBB4 (HER4), FGF2 (fibroblast growth factor 2), TGFB1 (TGF-beta-1), and a few matrix metallopeptidases such as MMP14 (MT1-MMP), were highly significantly down-regulated in PCX-OE cells (FIG. 8G-L). These results suggest that PCX acts as an upstream negative regulator of endometrial receptivity.

### PCX Tightens Cell-Cell Connection and Increases Epithelial Barrier Functions

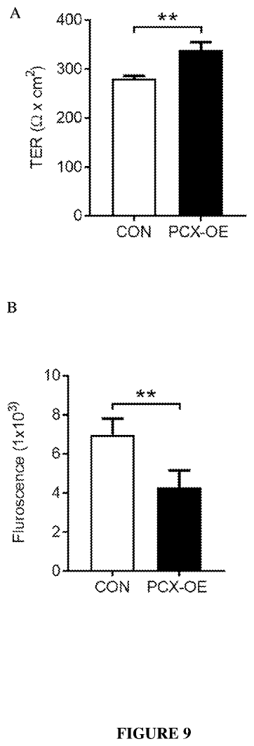

As a major functional feature of PCX-OE cells was inhibition of embryo invasion through the Ishikawa monolayer, immunofluorescence of cell junctional proteins E-cadherin, Wnt 7A, occludin, claudin 4 and ZO-1 was investigated. All these proteins were highly elevated in PCX-OE compared to control cells, consistent with their mRNA expression being significantly up-regulated. These staining results suggest that PCX-OE cells were connected to each other more tightly than control Ishikawa cells. To confirm this result, trans-epithelial electrical resistance (TER) across the monolayer, a biophysical measurement of epithelial barrier integrity, was measured. TER was significantly higher in PCX-OE than the control monolayer (FIG. 9A). The permeability of the monolayers for large molecules was also determined. FITC-labelled dextran (Mol wt 40 kDa) was added to the top of the monolayer and its flux to the bottom was quantified by measuring fluorescence signals in the bottom chamber. Dextran passage through the PCX-OE monolayer was highly significantly lower than that of the control (FIG. 9B), consistent with PCX-OE cells being joined more tightly. Collectively, these results suggest that PCX acts as a major epithelial cell sealant, up-regulating a range of cell junctional proteins to tighten cell-cell connection and to increase epithelial barrier functions. These data thus provide novel molecular and mechanistic insights into why the PCX-OE monolayer was more difficult for trophoblast spheroids and embryos to traverse through than the control Ishikawa monolayer.

Collectively, these studies suggest that PCX plays a critical regulatory role in governing epithelial junction and monolayer integrity. Consequently, PCX negatively regulates epithelial receptivity to embryo attachment as well as invasion, and PCX down-regulation in the endometrial LE is a functional necessity to establish endometrial receptivity.

### Example 10: Positive PCX Immunostaining in LE in the Putative Receptive Endometrium is Significantly Associated with Implantation Failure in IVF Patients

To further confirm that PCX in LE is a negative regulator of endometrial receptivity for embryo implantation, PCX in endometrial tissues from IVF patients was examined. In the current practice at many fertility centres, patients who fail to implant morphologically normal embryos after 2-3 cycles go through an “endometrial scratch biopsy” in the mid-secretory (putative receptive) phase before the next cycle. This biopsy is taken at this particular time when an embryo would normally be transferred, because of level 1 evidence that the scratch-associated injury leads to higher implantation rates in the next cycle, although its efficacy is controversial (van Hoogenhuijze et al., 2019; Frantz et al., 2019; Sar-Shalom et al., 2018: Nastri et al., 2015; Gnainsky et al., 2010). 86 such tissues that were biopsied previously at Monash IVF in Australia were obtained. These patients had transfer of a single high quality embryo in the next cycle and their implantation outcomes were known.

PCX in these endometrial tissues was examined by immunohistochemistry and the association between PCX staining in LE and implantation outcomes determined (Table 5). All tissues (n=86) showed positive PCX staining in the glands and blood vessels (data not shown). When LE staining was examined, 66 (77%) of these tissues were negative for PCX (PCX−), whereas the remaining 20 (23%) stained positively for PCX in >¼ of their LE cells which was defined as PCX+.

TABLE 5
Association of podocalyxin expression and implantation failure

Implantation outcomes

| | | |
|---|---|---|
| LE staining | Success | Failure |

| | | | |
|---|---|---|---|
| Total | 86 (100%) | 30 (35%) | 56 (65%) |
| PCX+ | 20 (23%) | 3 (15%) | 17 (85%) |
| PCX− | 66 (77%) | 27 (41%) | 39 (59%) |

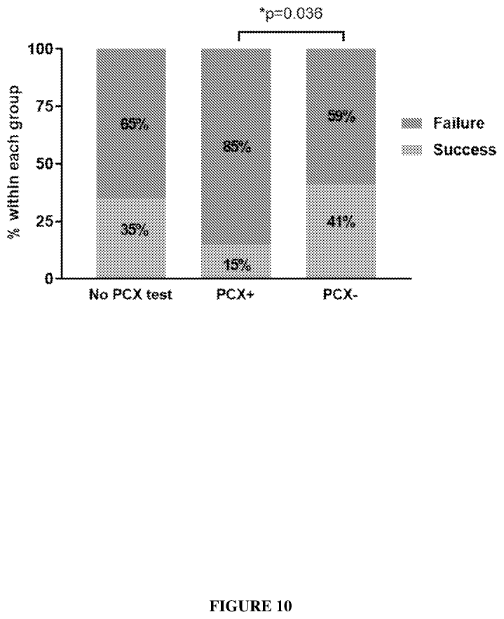

Implantation outcomes (6 week ultrasound) in the PCX− and PCX+ cohorts were then analysed separately (FIG. 10). In total, 30 (35%) of the entire cohort achieved successful implantation. In the PCX− group (66 in total), 27 (41%) were successful in implantation whereas the other 39 (59%) were not. In the PCX+ group (20 in total), however, implantation succeeded only in 3 (15%) and failed in 17 (85%). The difference between the two groups was statistically significant (p=0.036, Fisher's exact test).

These results provide important clinical evidence that PCX in LE is a significant negative regulator of embryo implantation. Moreover, this data in conjunction with the earlier functional studies, suggests that endometrial PCX positivity in LE may also contribute to implantation failure in IVF patients.

### Example 11: Regulation of Endometrial Epithelial PCX by MicroRNAs

The molecular mechanisms behind progesterone-induced down-regulation of PCX in the human endometrial epithelial cells for receptivity were investigated. Thirteen potential miRNAs that may target PCX were bioinformatically identified (Table 6) and their involvement in progesterone-induced PCX down-regulation in endometrial epithelial cells examined.

TABLE 6
Bioinformatically predicted miRNAs that may target PCX

| | |
|---|---|
| 1 | hsa-miR-199-5p |
| 2 | hsa-miR-152-3p |
| 3 | hsa-miR-145-5p |
| 4 | hsa-miR-219-5p |
| 5 | hsa-miR-34-5p |
| 6 | hsa-miR-181-5p |
| 7 | hsa-miR-144-3p |
| 8 | hsa-miR-802 |
| 9 | hsa-miR-125-5p |
| 10 | hsa-miR-143-3p |
| 11 | hsa-miR-202-5p |
| 12 | hsa-miR-124-3p |
| 13 | hsa-miR-15-5p |

Primary human endometrial epithelial cells were isolated and treated with estrogen (E, to mimic the proliferative phase) or estrogen plus progesterone (E+P, to mimic the secretory phase) for 96 h, and the levels of the above miRNAs were analysed by real-time RT-PCR. In addition, the control microRNA (hsa-miR-361-5p) was used.

Briefly, total RNA was extracted by mirVana™ miRNA Isolation Kits (Thermo Fisher Scientific) and RNA concentrations were determined using a NanoDrop™ 1000 Spectrophotometer (Thermo). The miRNA (10 ng) was reverse transcribed using TaqMan® Advanced miRNA cDNA Synthesis Kit (Thermo Fisher Scientific) as per the manufacturer's instructions. Real time RT-PCR was performed with miRNA assays (purchased from Thermo Fisher Scientific, Table 7), using QuantStudio 6 Flex Real-Time PCR System (Applied Biosystems) under the conditions specified in Table 8.

TABLE 8
Cycling conditions of real time RT-PCR analysis of microRNA

| | | Time | Number |
|---|---|---|---|
| | Temperature | (seconds) | of cycles |
| Stage 1 | 95° C. | 20 | 1 |
| Stage 2 | 95° C. | 1 | 40 |
| | 60° C. | 20 | |

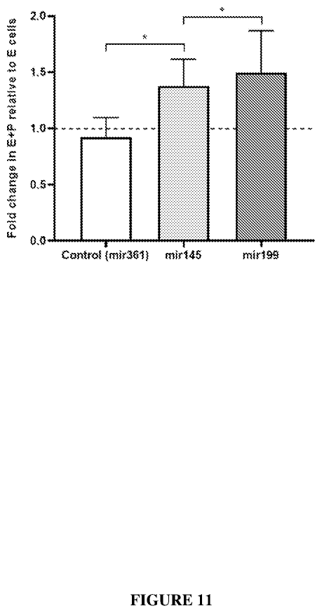

Some miRNAs showed no detection and many displayed variable and inconsistent changes following the E+P treatment. However, miR-145 and miR-199 showed moderate but consistent and significant up-regulation in E+P compared to cells treated with E alone (FIG. 11). The average fold change following E+P relative to E treatment was 1.38 for miR-145 and 1.50 for miR-199.

These results suggest that these two miRNAs may mediate the down-regulation of PCX by progesterone in the establishment of receptivity.

To confirm that these two miRNAs can directly down-regulate PCX, mimics of these miRNAs were transfected into a human endometrial epithelial Ishikawa cell line and the impact on the level of PCX expression examined.

Ishikawa cells were cultured overnight in a 12-well plate (3.0×10⁵ per well) in complete medium containing MEM (Thermo Fisher Scientific) supplemented with 10% FCS, 1% L-glutamine (Sigma) and 1% antibiotic-antimycotic. The following day, cells were replenished with Opti-MEM for transfection. Control and miRNA mimics (5 μm, all from Thermo Fisher Scientific) were transfected into Ishikawa cells using Lipofectamine RNAiMAX Transfection Reagent (Thermo Fisher Scientific) for 24, 48, 72 h respectively, and PCX mRNA levels were examined by real-time RT-PCR. Combination of the two miRNAs (5 μm each) was also tested.

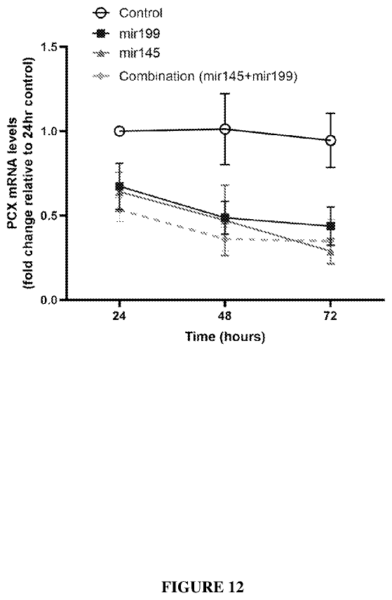

Following transfection, both miR-145 and miR-199 significantly down-regulated PCX mRNA (FIG. 12). Both miRNAs repressed PCX mRNA by ˜34% at 24 h, and this repression increased to ˜50˜60% and plateaued by 48-72 h. When the two miRNAs were transfected together, no synergistic effect was apparent.

These results confirm that both miR-145 and miR-199 can suppress PCX expression in endometrial epithelial cells.

TABLE 7
Details of miRNA assays

| | | | miRBase | | |
|---|---|---|---|---|---|
| | | | Accession | | SEQ ID |
| | Assay ID | Assay Name | Number | Sequence | NO: |
| 1 | 478231_mir | hsa-miR-199a-5p | MIMAT0000231 | CCCAGUGUUCAGACUACCUGUUC | 31 |
| 2 | 477921_mir | hsa-miR-152-3p | MIMAT0000438 | UCAGUGCAUGACAGAACUUGG | 32 |
| 3 | 477916_mir | hsa-miR-145-5p | MIMAT0000437 | GUCCAGUUUUCCCAGGAAUCCCU | 33 |
| 4 | 477980_mir | hsa-miR-219a-5p | MIMAT0000276 | UGAUUGUCCAAACGCAAUUCU | 34 |
| 5 | 478048_mir | hsa-miR-34a-5p | MIMAT0000255 | UGGCAGUGUCUUAGCUGGUUGU | 35 |
| 6 | 477857_mir | hsa-mir-181a-5p | MIMAT0000256 | AACAUUCAACGCUGUCGGUGAGU | 36 |
| 7 | 477913_mir | hsa-miR-144-3p | MIMAT0000436 | UACAGUAUAGAUGAUGUACU | 37 |
| 8 | 479181_mir | hsa-miR-802 | MIMAT0004185 | CAGUAACAAAGAUUCAUCCUUGU | 38 |
| 9 | 477885_mir | hsa-miR-125b-5p | MIMAT0000423 | UCCCUGAGACCCUAACUUGUGA | 39 |
| 10 | 477912_mir | hsa-miR-143-3p | MIMAT0000435 | UGAGAUGAAGCACUGUAGCUC | 40 |
| 11 | 478755_mir | hsa-miR-202-5p | MIMAT0002810 | UUCCUAUGCAUAUACUUCUUUG | 41 |
| 12 | 478958_mir | hsa-miR-506-3p | MIMAT0002878 | UAAGGCACCCUUCUGAGUAGA | 42 |
| | | (124-3p.2) | | | |
| 13 | 477860_mir | hsa-miR-16-5p | MIMAT0000069 | UAGCAGCACGUAAAUAUUGGCG | 43 |
| | | (15-5p) | | | |
| 14 | 478056_mir | hsa-miR-361-5p | MIMAT0000703 | UUAUCAGAAUCUCCAGGGGUAC | 44 |
| | | (Control) | | | |

### REFERENCES

Abou-Elkacem et al., (2015) European Journal of Radiology, 84:1685-1693.
Achache et al., (2006) Human Reproduction Update, 12:731-746.
Altmäe et al., (2010) Mol. Hum. Reprod., 16:178-187.
Altmäe et al., (2017) Scientific Reports, 7:10077.
Andrews et al., (2010) http://www.bioinformatics.babraham.ac.uk/projects/fastqc
Aplin et al., (2017) Journal of Cell Science, 130:15-22.
Ashary et al., (2018) Endocrinology, 159:1188-1198.
Ausubel et al., (ed.), Current Protocols in Molecular Biology, 1988, John Wiley and Sons, Inc.
Ausubel et al, (ed.), Short Protocols in Molecular Biology, 1995, Wiley.
Bischof et al., (1996) Human Reproduction Update, 2:262-270.
Brady et al., (1987) Phil. Trans. R. Soc. Land., 316: 143-160.
Bresslauer et al., (1986) Proc. Natl. Acad. Sci., 83:3746-3750.
Brown (ed.), Essential Molecular Biology: A Practical Approach, 1991, IRL Press, Volumes 1 and 2.
Casper et al., (2016) Fertility and Sterility, 105:867-872.
Chambers et al., (2016) Human Reproduction, 31:2632-2641.
Chen et al., (2017) Journal of Hypertension, 35:2287-2294.
Cheung et al., (2011) Oncogene, 30:3404.
Coligan et al., (ed.), Current Protocols in Immunology, 1991, John Wiley & Sons.
Cole et al., Monoclonal Antibodies in Cancer Therapy, 1985 Allen R. Bliss, Inc., pages 77-96.
Craciunas et al., (2019) Human Reproduction Update, 25:202-223.
Cuello, ASIN 0471900524, 1984, John Wiley and Sons.
Delaney et al., (2016) PLOS ONE, 11:e0159114.
Dieffenbach et al., (ed.), PCR Primer: A Laboratory Manual, 1995, Cold Spring Harbor Laboratories.
Dobin et al., (2013) Bioinformatics, 29(1), 15-21.
Dolgaley (2018) R package version 6.2.1. https://CRAN.R-project.org/package=msigdbr
Duijkers et al., (2018) Human Reproduction, 33:2131-2140.
Durinck et al (2009) Nature Protocols, 4(8), pp. 1184-1191
Dyer et al., (2016) Human Reproduction, 31:1588-1609.
El-Sahwi et al., (2010) Molecular Cancer Therapeutics, 9:57-66.
Evans et al., (2014) Fertility and Sterility, 102:307-317.e307.
Evans et al., (2016) Nature Reviews Endocrinology, 12:654-667.
Favreau et al., (2012) American Journal of Hematology, 87:442-446.
Frantz et al., (2019) Human Reproduction, 34:92-99.
Fritz et al., (2017) Human Reproduction, 32:1903-1914.
Gait (ed), Oligonucleotide Synthesis: A Practical Approach, 1984, IRL Press.
Gardner et al., (1999) Curr Opin Obstet Gynecol., 11:307-311.
Garrido-Gómez et al., (2013) Fertility and Sterility, 99:1078-1085.
Glover et al., (ed.), DNA Cloning: A Practical Approach, 1995 and 1996, IRL Press, Volumes 1 to 4.
Gnainsky et al., (2010) Fertility and Sterility, 94:2030-2036.
Griesinger et al., (2018) Human Reproduction, 33:2212-2221.
Grifo et al., (2013) Journal of Assisted Reproduction and Genetics, 30:259-264.
Haouzi et al., (2012) Reproductive BioMedicine Online, 24:23-34.
Harlow et al., (ed.), Antibodies: A Laboratory Manual, 1988, Cold Spring Harbour Laboratory.
Hartmann et al. (ed.), Manual of Antisense Methodology, 1999, Kluwer.
Heng et al., (2015) The FASEB Journal, 29:4011-4022.
Ho et al., (2012) Fertility and Sterility, 97:974-978.
Huse et al., (1989) Science 246:1275.
James et al., (2012) Placenta, 33:327-334.
Kershaw et al., (1997) Journal of Biological Chemistry, 272:15708-15714.
Kliman et al., (2019) Fertility and Sterility, 111:618-628.
Kohler et al., (1976) Eur. J. Immunol., 6:511-519.
Kolde (2019) R package version 1.0.12. https://CRAN.R-project.org/package=pheatmap
Koot et al., (2011) Human Reproduction, 26:2636-2641.
Law et al (2014) Genome Biology, 15(2), R29.
Lee et al., (2004) Reproduction, 128:679-695.
Lessey et al., (1992) The Journal of Clinical Investigation, 90:188-195.
Lessey et al., (1994) Fertility and Sterility, 62:497-506.
Lessey, (2011) Fertility and Sterility, 96:522-529.
Lessey et al., (2019) Fertility and Sterility, 111:611-617.
Liao et al (2014) Bioinformatics, 30(7), 923-930.
Liberzon et al (2015) Cell Systems, 1(6), 417-425.
Margalioth et al., (2006) Human Reproduction, 21:3036-3043.
Marwood et al., (2009) Endocrinology, 150:2915-23.
Mastenbroek et al., (2011) Human Reproduction Update, 17:454-466.
McCarthy et al (2012) Nucleic Acids Research, 40(10), pp. 4288-4297.
Mendoza et al., (1999) Biotechniques, 27:778-788.
Nastri et al., (2015) Cochrane Database Syst Rev., Art. No.: CD009517.
Nie et al., (2019) Cambridge University Press, 2019: 10-18.
Nielsen et al., (2009) Journal of American Society of Nephrology, 20:1669-1676.
Norwitz et al., (2001) New England Journal Medicine, 345:1400-1408.
Novakovic et al., (2017) Scientific Reports, 7:4523-4523.
Noyes et al., (1975) Am J Obstet Gynecol., 122:262-263.
Park et al., (2000) Mol Hum Reprod., 6:252-257.
Paule et al., (2012) Human Reproduction, 27:2766-2774.
Perbal, A Practical Guide to Molecular Cloning, 1984, John Wiley and Sons.
Revel, (2012) Fertility and Sterility, 97:1028-1032.
Robins, (1991) Advances in Biosensors, 1:229-256.
Robinson et al (2009) Bioinformatics, 26(1), 139-140.
Robinson et al (2010) Genome biology, 11(3), p.R25.
Salamonsen et al., (2009) Reprod Fertil Dev, 21:923-934
Sambrook et al., Molecular Cloning: A Laboratory Manual, 1989, Cold Spring Harbor Laboratory Press.
Santa Lucia, (1995) Proc. Natl. Acad. Sci., 95:1460-1465.
Sar-Shalom et al., (2018) Human Reproduction Update, 25:95-113.
Sarani et al., (1999) Human Reproduction, 14:3101-3106.
Scopes, Protein Purification: Principles and Practice, 1994, Springer Verlag.
Schubert et al (2016) BMC Research Notes, 9(1), 88.
Sharkey et al., (2003) Best practice & research clinical obstetrics & gynecology, the management of subfertility, 17:289-307.
Sharkey et al., (2013) Reproductive BioMedicine Online, 27:453-460.
Smith et al., (2019) Fertility and Sterility, 111:641-649.
Thomsen et al., (2018) Human Reproduction, 33:1506-1516.
Tsuruta et al., (2014) PLOS ONE, 9:e86642.
van Hoogenhuijze et al., (2019) Human Reproduction Open, 2019:1-18.
von Grothusen et al., (2014) American Journal of Reproductive Immunology, 72:148-157.
Wallace et al., (2017) Placenta, 52:62-70.
Willmann et al., (2017) Journal of Clinical Oncology, 35:2133-2140.
Wisniewski et al., (2009) Nature Methods, 6:359.
Yeh et al., (2015) PLOS ONE, 10:e0129681.
Yu et al (2012) OMICS, 16(5), 284-287.

1. A method of predicting endometrial receptivity for embryo implantation in a subject, the method comprising determining a level of podocalyxin in endometrial epithelial cells in the subject.

2. The method of claim 1, wherein determining the level of podocalyxin comprises determining the amount and/or distribution pattern of podocalyxin protein, and/or determining the amount of nucleic acid molecules encoding podocalyxin, in the endometrial epithelial cells.

3. The method of claim 2, wherein the nucleic acid molecules are mRNA.

4. The method of any one of claims 1 to 3, wherein the method further comprises comparing the level of podocalyxin in the subject to a level of podocalyxin in endometrial epithelial cells in at least one reference.

5. The method of claim 4, wherein the method comprises determining (a) if the level of the podocalyxin in the subject is higher than the level of the podocalyxin in the reference, or (b) if the level of the podocalyxin in the subject is lower than the level of podocalyxin in the reference.

6. The method of any one of claims 1 to 5, wherein the endometrial epithelial cells are luminal epithelial cells and/or glandular epithelial cells.

7. The method of claim 6, wherein:
(i) a lower level of podocalyxin in luminal epithelial cells and a higher level of podocalyxin in glandular epithelial cells of the subject is indicative of endometrial epithelial receptivity; or
(ii) a higher level of podocalyxin in luminal epithelial cells and a higher level of podocalyxin in glandular epithelial cells of the subject is indicative of pre-endometrial epithelial receptivity; or
(iii) a lower level of podocalyxin in luminal epithelial cells and a lower level of podocalyxin in glandular epithelial cells of the subject is indicative of post-endometrial epithelial receptivity.

8. The method of any one of claims 1 to 7, wherein the method comprises using an antibody or aptamer that specifically binds podocalyxin to determine the level of podocalyxin.

9. The method of claim 8, wherein the antibody or aptamer is conjugated to a detectable label.

10. The method of claim 9, wherein the detectable label is selected from the group consisting of a radiolabel, an enzyme, a fluorescent label, a luminescent label, a bioluminescent label, a magnetic label, a prosthetic group, a contrast agent and an ultrasound agent.

11. The method of claim 10, wherein the ultrasound agent is a microbubble-releasing agent.

12. The method of any one of claims 1 to 7, wherein determining the level of podocalyxin comprises determining the level of a downstream regulator of progesterone and/or an upstream regulator of podocalyxin.

13. The method of claim 12, wherein the downstream regulator of progesterone and/or an upstream regulator of podocalyxin is a microRNA.

14. The method of claim 13, wherein the microRNA is miR-199 or miR-145.

15. The method of any one of claims 1 to 14, wherein the method comprises performing an immunohistochemical assay, in situ hybridization, flow cytometry, an enzyme-linked immunosorbent assay, western blot, real-time reverse transcription polymerase chain reaction (RT-PCR) or ultrasound molecular imaging

16. The method of any one of claims 1 to 15, wherein the method is performed on endometrial epithelial cells in vitro or ex vivo.

17. The method of claim 16, wherein the method is performed on endometrial epithelial cells obtained from the subject in a biological sample.

18. The method of claim 17, wherein the biological sample is selected from the group consisting of an endometrial biopsy, a uterine fluid sample and a vaginal fluid sample.

19. The method of any one of claims 1 to 18, wherein the subject has been previously treated with a composition comprising progesterone, progestogen or an analog or combinations thereof.

20. The method of any one of claims 1 to 19, wherein the level of podocalyxin is determined in at least one biological sample and at least one time point during a cycle.

21. The method of any one of claims 1 to 20, further comprising implantation of an embryo into the subject.

22. The method of any one of claims 1 to 21, wherein the level of podocalyxin is determined in a first cycle of the subject and an embryo is implanted in a subsequent cycle of the subject.

23. A method of detecting infertility in a subject, the method comprising determining a level of podocalyxin in endometrial epithelial cells in the subject.

24. A method of diagnosis and prognosis of infertility in a subject, the method comprising determining a level of podocalyxin in endometrial epithelial cells in the subject.

25. The method of claim 23 or 24, wherein the level of podocalyxin is determined in at least one biological sample and at least one time point during a cycle.

26. A method of monitoring endometrial epithelial receptivity and predicting optimal endometrial epithelial receptivity for embryo implantation in a subject, the method comprising determining a level of podocalyxin in endometrial epithelial cells in the subject at one or more time points.

27. A method of improving endometrial epithelial receptivity for embryo implantation in a subject, the method comprising determining a level of podocalyxin in endometrial epithelial cells in the subject, and based on the level of podocalyxin in the cells, administering to the subject a compound in an amount sufficient to reduce the level of podocalyxin in the endometrial epithelial cells.

28. A method of assessing effectiveness of a compound on improving endometrial epithelial receptivity for embryo implantation in a subject, the method comprising determining a level of podocalyxin in endometrial epithelial cells in the subject, wherein the subject has previously received treatment with the compound.

29. A method of optimising treatment with a compound to improve endometrial epithelial receptivity for embryo implantation in a subject, the method comprising administering to the subject a compound, determining a level of podocalyxin in endometrial epithelial cells in the subject and optionally, based on the level of podocalyxin, modifying the treatment to the subject.

30. The method of claim 29, wherein the modification is one or more or all of dose, type of compound and/or route of administered.

31. The method of any one of claims 27 to 30, wherein the compound is selected from the group consisting of progesterone, progestogen, or an analog thereof, an antisense polynucleotide, a catalytic nucleic acid, an interfering RNA, a siRNA, a microRNA and combinations thereof.


SEQUENCE LISTING (44 sequences)

SEQ ID NO: 1 - DNA, 20 bp, Artificial Sequence
  PODXL (PCX) forward primer
gagcagtcaaagccaccttc

SEQ ID NO: 2 - DNA, 20 bp, Artificial Sequence
  PODXL (PCX) reverse primer
tggtcccctagcttcatgtc

SEQ ID NO: 3 - DNA, 22 bp, Artificial Sequence
  CDH1 forward primer
gaaggtgacagagcctctggat

SEQ ID NO: 4 - DNA, 23 bp, Artificial Sequence
  CDH1 reverse primer
gatcggttaccgtgatcaaaatc

SEQ ID NO: 5 - DNA, 21 bp, Artificial Sequence
  TJP1 forward primer
gggaacaacatacagtgacgc

SEQ ID NO: 6 - DNA, 20 bp, Artificial Sequence
  TJP1 reverse primer
ccccactctgaaaatgagga

SEQ ID NO: 7 - DNA, 20 bp, Artificial Sequence
  CLDN4 forward primer
ccccgagagagagtgccctg

SEQ ID NO: 8 - DNA, 21 bp, Artificial Sequence
  CLDN4 reverse primer
agcgtccacgggagttgagga

SEQ ID NO: 9 - DNA, 20 bp, Artificial Sequence
  OCLN forward primer
ctctctcagccagcctactc

SEQ ID NO: 10 - DNA, 20 bp, Artificial Sequence
  OCLN reverse primer
gttccatagcctctgtccca

SEQ ID NO: 11 - DNA, 19 bp, Artificial Sequence
  WNT7A forward primer
tgcccggactctcatgaac

SEQ ID NO: 12 - DNA, 20 bp, Artificial Sequence
  WNT7A reverse primer
gtgtggtccagcacgtcttg

SEQ ID NO: 13 - DNA, 20 bp, Artificial Sequence
  LEFTY2 forward primer
ctggacctcagggactatgg

SEQ ID NO: 14 - DNA, 20 bp, Artificial Sequence
  LEFTY2 reverse primer
tcaatgtacatctcctggcg

SEQ ID NO: 15 - DNA, 20 bp, Artificial Sequence
  LIF forward primer
tgccaatgccctctttattc

SEQ ID NO: 16 - DNA, 20 bp, Artificial Sequence
  LIF reverse primer
gttgacagcccagcttcttc

SEQ ID NO: 17 - DNA, 22 bp, Artificial Sequence
  CSF1 forward primer
tagccacatgattgggagtgga

SEQ ID NO: 18 - DNA, 25 bp, Artificial Sequence
  CSF1 reverse primer
ctcaaatgtaatttggcacgaggtc

SEQ ID NO: 19 - DNA, 22 bp, Artificial Sequence
  ERBB4 forward primer
gatgatcgtatgaagcttccca

SEQ ID NO: 20 - DNA, 24 bp, Artificial Sequence
  ERBB4 reverse primer
cggtatacaaactggttcctattc

SEQ ID NO: 21 - DNA, 19 bp, Artificial Sequence
  FGF2 forward primer
cggatgggggtagtgagca

SEQ ID NO: 22 - DNA, 20 bp, Artificial Sequence
  FGF2 reverse primer
atcttgaggtggaagggtct

SEQ ID NO: 23 - DNA, 21 bp, Artificial Sequence
  TGFB1 forward primer
caacaattcctggcgatacct

SEQ ID NO: 24 - DNA, 21 bp, Artificial Sequence
  TGFB1 reverse primer
gctaaggcgaaagccctcaat

SEQ ID NO: 25 - DNA, 21 bp, Artificial Sequence
  MMP14 forward primer
gcagaagttttacggcttgca

SEQ ID NO: 26 - DNA, 21 bp, Artificial Sequence
  MMP14 reverse primer
tcgaacattggccttgatctc

SEQ ID NO: 27 - DNA, 20 bp, Artificial Sequence
  YWHAZ forward primer
ccgccaggacaaaccagtat

SEQ ID NO: 28 - DNA, 24 bp, Artificial Sequence
  YWHAZ reverse primer
acttttggtacattgtggcttcaa

SEQ ID NO: 29 - DNA, 20 bp, Artificial Sequence
  18S forward primer
cggctaccacatccaaggaa

SEQ ID NO: 30 - DNA, 18 bp, Artificial Sequence
  18S reverse primer
gctggaattaccgcggct

SEQ ID NO: 31 - DNA, 23 bp, Artificial Sequence
  hsa-miR-199a-5p
cccaguguucagacuaccuguuc

SEQ ID NO: 32 - DNA, 21 bp, Artificial Sequence
  hsa-miR-152-3p
ucagugcaugacagaacuugg

SEQ ID NO: 33 - DNA, 23 bp, Artificial Sequence
  hsa-miR-145-5p
guccaguuuucccaggaaucccu

SEQ ID NO: 34 - DNA, 21 bp, Artificial Sequence
  hsa-miR-219a-5p
ugauuguccaaacgcaauucu

SEQ ID NO: 35 - DNA, 22 bp, Artificial Sequence
  hsa-miR-34a-5p
uggcagugucuuagcugguugu

SEQ ID NO: 36 - DNA, 23 bp, Artificial Sequence
  hsa-mir-181a-5p
aacauucaacgcugucggugagu

SEQ ID NO: 37 - DNA, 20 bp, Artificial Sequence
  hsa-miR-144-3p
uacaguauagaugauguacu

SEQ ID NO: 38 - DNA, 23 bp, Artificial Sequence
  hsa-miR-802
caguaacaaagauucauccuugu

SEQ ID NO: 39 - DNA, 22 bp, Artificial Sequence
  hsa-miR-125b-5p
ucccugagacccuaacuuguga

SEQ ID NO: 40 - DNA, 21 bp, Artificial Sequence
  hsa-miR-143-3p
ugagaugaagcacuguagcuc

SEQ ID NO: 41 - DNA, 22 bp, Artificial Sequence
  hsa-miR-202-5p
uuccuaugcauauacuucuuug

SEQ ID NO: 42 - DNA, 21 bp, Artificial Sequence
  hsa-miR-506-3p (124-3p.2)
uaaggcacccuucugaguaga

SEQ ID NO: 43 - DNA, 22 bp, Artificial Sequence
  hsa-miR-16-5p (15-5p)
uagcagcacguaaauauuggcg

SEQ ID NO: 44 - DNA, 22 bp, Artificial Sequence
  hsa-miR-361-5p (Control)
uuaucagaaucuccagggguac